SEQUENCE LISTING (148 sequences)

SEQ ID NO: 1 - protein, 117 aa, Artificial Sequence
  16B8.C8 VH
EVQLQESGPGLVQPSQSLSITCTVSGFSLTNYGLHWVRQSPGKGLEWLGVIWSGGKTDYNTPFKSRLSISKDISKNQVFFKMNSLQPNDTAIYFCAKYDYDDSLDYWGQGTSVTVSS

SEQ ID NO: 2 - protein, 107 aa, Artificial Sequence
  16B8.C8 VL
DIQMNQSPSSLSASLGDTIAITCHASQNINFWLSWYQQKPGNIPKLLIYEASNLHTGVPSRFSGSGSGTRFTLTISSLQPEDIATYYCQQSHSYPLTFGQGTKLEIK

SEQ ID NO: 3 - protein, 244 aa, Artificial Sequence
  scFv-1292 (VH-VL)
QLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCAKYDYDDSLDYWGQGTLVTVSSGGGGSGGGGSGGGGSGGGGSDIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIK

SEQ ID NO: 4 - protein, 5 aa, Artificial Sequence
  16B8.C8 VH CDR2
WSGGK

SEQ ID NO: 5 - protein, 9 aa, Artificial Sequence
  16B8.C8 VH CDR3
YDYDDSLDY

SEQ ID NO: 6 - protein, 11 aa, Artificial Sequence
  16B8.C8 VL CDR1
HASQNINFWLS

SEQ ID NO: 7 - protein, 7 aa, Artificial Sequence
  16B8.C8 VL CDR2
EASNLHT

SEQ ID NO: 8 - protein, 9 aa, Artificial Sequence
  16B8.C8 VL CDR3
QQSHSYPLT

SEQ ID NO: 9 - protein, 117 aa, Artificial Sequence
  Humanized 16B8.C8 in scFv-1292/scFv-1301 VH
QLQLQESGPGLVKPSETLSLTCTVSGFSLTNYGLHWIRQPPGKGLEWIGVIWSGGKTDYNPSLKSRVTISKDTSKNQFSLKLSSVQANDTAVYYCAKYDYDDSLDYWGQGTLVTVSS

SEQ ID NO: 10 - protein, 107 aa, Artificial Sequence
  Humanized 16B8.C8 in scFv-1292/scFv-1301 VL
DIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGQGTKLEIK

SEQ ID NO: 11 - protein, 7 aa, Artificial Sequence
  Humanized 16B8.C8 in scFv-1292/scFv-1301 VH CDR1
GFSLTNY

SEQ ID NO: 12 - protein, 244 aa, Artificial Sequence
  scFv-1301 (VL-VH)
DIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCAKYDYDDSLDYWGQGTLVTVSS

SEQ ID NO: 13 - protein, 117 aa, Artificial Sequence
  Humanized 16B8.C8 in scFv-1293/scFv-1302 VH
QLQLQESGPGLVKPSETLSLTCTVSGFSLTNYGLHWIRQPPGKGLEWIGVIWSGGKTDYNPSLKSRVTISVDTSKNQFSLKLSSVQANDTAVYYCAKYDYDDSLDYWGQGTLVTVSS

SEQ ID NO: 14 - protein, 120 aa, Artificial Sequence
  Humanized 9F11.B7 in AB0192/AB0186 VH
EVQLVESGGGVVQPGGSLRLSCAASGFTFNAFGMHWVRQAPGKGLEWVAFISSGSTSIYYANTVKGRFTISRDNSKNTLYLQMNSLRAEDTAVYYCARDGYPTGGAMDYWGQGTSVTVSS

SEQ ID NO: 15 - protein, 244 aa, Artificial Sequence
  scFv-1293 (VH-VL)
QLQLQESGPGLVKPSETLSLTCTVSGFSLTNYGLHWIRQPPGKCLEWIGVIWSGGKTDYNPSLKSRVTISVDTSKNQFSLKLSSVQANDTAVYYCAKYDYDDSLDYWGQGTLVTVSSGGGGSGGGGSGGGGSGGGGSDIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIK

SEQ ID NO: 16 - protein, 244 aa, Artificial Sequence
  scFv-1302 (VL-VH)
DIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISVDTSKNQFSLKLSSVQANDTAVYYCAKYDYDDSLDYWGQGTLVTVSS

SEQ ID NO: 17 - protein, 107 aa, Artificial Sequence
  Humanized 16B8.C8 consensus -VL
  misc_feature (43)..(43): Xaa can be Ala or Ile
  misc_feature (70)..(70): Xaa can be Asp or Arg
  misc_feature (83)..(83): Xaa can be Phe or Ile
  misc_feature (100)..(100): Xaa can be Gln or Gly
DIQMTQSPSSLSASVGDRVTITCHASQNINFWLSWYQQKPGKXPKLLIYEASNLHTGVPSRFSGSGSGTXFTLTISSLQPEDXATYYCQQSHSYPLTFGXGTKLEIK

SEQ ID NO: 18 - protein, 17 aa, Artificial Sequence
  VL CDR1
KSSQSLLWNVNQNNYLV

SEQ ID NO: 19 - protein, 244 aa, Artificial Sequence
  scFv-1294 (VH-VL)
QLQLQESGPGLVKPSETLSLTCTVSGFSLTNYGLHWIRQPPGKCLEWIGVIWSGGKTDYNPSLKSRVTISKDTSKNQFSLKLSSVTANDTAVYYCAKYDYDDSLDYWGQGTLVTVSSGGGGSGGGGSGGGGSGGGGSDIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIK

SEQ ID NO: 20 - protein, 244 aa, Artificial Sequence
  scFv-1303 (VL-VH)
DIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISKDTSKNQFSLKLSSVTANDTAVYYCAKYDYDDSLDYWGQGTLVTVSS

SEQ ID NO: 21 - protein, 226 aa, Homo sapiens
  wild-type human IgG1 Fc sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 119 aa, Artificial Sequence
  15A10.G8 VH
EVQLQESGAELVRSGASIKLSCAASAFNIKDYFIHWVRQRPDQGLEWIGWIDPENDDTEYAPKFQDKATMTADTSSNTAYLQLSSLTSADTAVYYCNALWSRGGYFDYWGQGTTLTVSS

SEQ ID NO: 23 - protein, 244 aa, Artificial Sequence
  scFv-1295 (VH-VL)
QLQLQESGPGLVKPSETLSLTCTVSGFSLTNYGLHWIRQPPGKCLEWIGVIWSGGKTDYNPSLKSRVTISKDTSKNQFSLKLSSVQAADTAVYYCAKYDYDDSLDYWGQGTLVTVSSGGGGSGGGGSGGGGSGGGGSDIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIK

SEQ ID NO: 24 - protein, 244 aa, Artificial Sequence
  scFv-1304 (VL-VH)
DIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISKDTSKNQFSLKLSSVQAADTAVYYCAKYDYDDSLDYWGQGTLVTVSS

SEQ ID NO: 25 - protein, 106 aa, Artificial Sequence
  15A10.G8 VL
EVLLTQSPAIIAASPGEKVTITCSARSSVSYMSWYQQKPGSSPKIWIYGISKLASGVPARFSGSGSGTYFSFTINNLEAEDVATYYCQQRSYYPFTFGSGTKLEIK

SEQ ID NO: 26 - protein, 7 aa, Artificial Sequence
  VH CDR1
AFNIKDY

SEQ ID NO: 27 - protein, 244 aa, Artificial Sequence
  scFv-1296 (VH-VL)
QLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCARYDYDDSLDYWGQGTLVTVSSGGGGSGGGGSGGGGSGGGGSDIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIK

SEQ ID NO: 28 - protein, 244 aa, Artificial Sequence
  scFv-1305 (VL-VH)
DIQMTQSPSSLSASVGDRVTITCHASQNINFWLSWYQQKPGKIPKLLIYEASNLHTGVPSRFSGSGSGTRFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCARYDYDDSLDYWGQGTLVTVSS

SEQ ID NO: 29 - protein, 120 aa, Artificial Sequence
  Humanized 9F11.B7 in AB0191/AB0185 VH
EVQLVESGGGVVQPGGSLRLSCAASGFTFNSFGMHWVRQAPGKGLEWVAFISSGSTSIYYANTVKGRFTISRDNSKNTLYLQMNSLRAEDTAVYYCARDGYPTGGAMDYWGQGTSVTVSS

SEQ ID NO: 30 - protein, 107 aa, Artificial Sequence
  Humanized 16B8.C8 in scFv-1297/scFv-1306 VL
DIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGQGTKLEIK

SEQ ID NO: 31 - protein, 244 aa, Artificial Sequence
  scFv-1297 (VH-VL)
QLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCAKYDYDDSLDYWGQGTLVTVSSGGGGSGGGGSGGGGSGGGGSDIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGCGTKLEIK

SEQ ID NO: 32 - protein, 244 aa, Artificial Sequence
  scFv-1306 (VL-VH)
DIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCAKYDYDDSLDYWGQGTLVTVSS

SEQ ID NO: 33 - protein, 5 aa, Artificial Sequence
  VH CDR2
WSGGS

SEQ ID NO: 34 - protein, 107 aa, Artificial Sequence
  Humanized 16B8.C8 in scFv-1298/scFv-1307 VL
DIQMTQSPSSLSASVGDRVTITCHASQNINFWLSWYQQKPGKIPKLLIYEASNLHTGVPSRFSGSGSGTDFTLTISSLQPEDIATYYCQQSHSYPLTFGQGTKLEIK

SEQ ID NO: 35 - protein, 244 aa, Artificial Sequence
  scFv-1298 (VH-VL)
QLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCAKYDYDDSLDYWGQGTLVTVSSGGGGSGGGGSGGGGSGGGGSDIQMTQSPSSLSASVGDRVTITCHASQNINFWLSWYQQKPGKIPKLLIYEASNLHTGVPSRFSGSGSGTDFTLTISSLQPEDIATYYCQQSHSYPLTFGCGTKLEIK

SEQ ID NO: 36 - protein, 244 aa, Artificial Sequence
  scFv-1307 (VL-VH)
DIQMTQSPSSLSASVGDRVTITCHASQNINFWLSWYQQKPGKIPKLLIYEASNLHTGVPSRFSGSGSGTDFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCAKYDYDDSLDYWGQGTLVTVSS

SEQ ID NO: 37 - protein, 6 aa, Artificial Sequence
  15A10.G8 VH - CDR2
DPENDD

SEQ ID NO: 38 - protein, 107 aa, Artificial Sequence
  Humanized 16B8.C8 in scFv-1299/scFv-1308 VL
DIQMTQSPSSLSASVGDRVTITCHASQNINFWLSWYQQKPGKIPKLLIYEASNLHTGVPSRFSGSGSGTRFTLTISSLQPEDFATYYCQQSHSYPLTFGQGTKLEIK

SEQ ID NO: 39 - protein, 244 aa, Artificial Sequence
  scFv-1299 (VH-VL)
QLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCAKYDYDDSLDYWGQGTLVTVSSGGGGSGGGGSGGGGSGGGGSDIQMTQSPSSLSASVGDRVTITCHASQNINFWLSWYQQKPGKIPKLLIYEASNLHTGVPSRFSGSGSGTRFTLTISSLQPEDFATYYCQQSHSYPLTFGCGTKLEIK

SEQ ID NO: 40 - protein, 244 aa, Artificial Sequence
  scFv-1308 (VL-VH)
DIQMTQSPSSLSASVGDRVTITCHASQNINFWLSWYQQKPGKIPKLLIYEASNLHTGVPSRFSGSGSGTRFTLTISSLQPEDFATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISKDTSKNQFSLKLSSVQANDTAVYYCAKYDYDDSLDYWGQGTLVTVSS

SEQ ID NO: 41 - protein, 117 aa, Artificial Sequence
  Humanized 16B8.C8 in scFv-1300/scFv-1309 VH
QLQLQESGPGLVKPSETLSLTCTVSGFSLTNYGLHWIRQPPGKGLEWIGVIWSGGKTDYNPSLKSRVTISVDTSKNQFSLKLSSVTAADTAVYYCARYDYDDSLDYWGQGTLVTVSS

SEQ ID NO: 42 - protein, 107 aa, Artificial Sequence
  Humanized 16B8.C8 in scFv-1300/scFv-1309 VL
DIQMTQSPSSLSASVGDRVTITCHASQNINFWLSWYQQKPGKAPKLLIYEASNLHTGVPSRFSGSGSGTDFTLTISSLQPEDFATYYCQQSHSYPLTFGQGTKLEIK

SEQ ID NO: 43 - protein, 244 aa, Artificial Sequence
  scFv-1300 (VH-VL)
QLQLQESGPGLVKPSETLSLTCTVSGFSLTNYGLHWIRQPPGKCLEWIGVIWSGGKTDYNPSLKSRVTISVDTSKNQFSLKLSSVTAADTAVYYCARYDYDDSLDYWGQGTLVTVSSGGGGSGGGGSGGGGSGGGGSDIQMTQSPSSLSASVGDRVTITCHASQNINFWLSWYQQKPGKAPKLLIYEASNLHTGVPSRFSGSGSGTDFTLTISSLQPEDFATYYCQQSHSYPLTFGCGTKLEIK

SEQ ID NO: 44 - protein, 244 aa, Artificial Sequence
  scFv-1309 (VL-VH)
DIQMTQSPSSLSASVGDRVTITCHASQNINFWLSWYQQKPGKAPKLLIYEASNLHTGVPSRFSGSGSGTDFTLTISSLQPEDFATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISVDTSKNQFSLKLSSVTAADTAVYYCARYDYDDSLDYWGQGTLVTVSS

SEQ ID NO: 45 - protein, 117 aa, Artificial Sequence
  Humanized 16B8.C8 in scFv-1295/scFv-1304 VH
QLQLQESGPGLVKPSETLSLTCTVSGFSLTNYGLHWIRQPPGKGLEWIGVIWSGGKTDYNPSLKSRVTISKDTSKNQFSLKLSSVQAADTAVYYCAKYDYDDSLDYWGQGTLVTVSS

SEQ ID NO: 46 - protein, 11 aa, Artificial Sequence
  VL CDR3
QHNHGSFLPYT

SEQ ID NO: 47 - protein, 244 aa, Artificial Sequence
  scFv-1602 (VH-VL)
QLQLQESGPGLVKPSETLSLTCTVSGFSLTNYGLHWIRQPPGKCLEWIGVIWSGGKTDYNPSLKSRVTISKDTSKNQFSLKLSSVQAADTAVYYCAKYDYDDSLDYWGQGTLVTVSSGGGGSGGGGSGGGGSGGGGSDIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGCGTKLEIK

SEQ ID NO: 48 - protein, 244 aa, Artificial Sequence
  scFv-2061 (VL-VH)
DIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWSGGKTDYNPSLKSRVTISKDTSKNQFSLKLSSVQAADTAVYYCAKYDYDDSLDYWGQGTLVTVSS

SEQ ID NO: 49 - protein, 117 aa, Artificial Sequence
  Humanized 16B8.C8 consensus - VH
  misc_feature (71)..(71): Xaa can be Val or Lys
  misc_feature (86)..(86): Xaa can be Thr or Gln
  misc_feature (88)..(88): Xaa can be Ala or Asn
  misc_feature (97)..(97): Xaa can be Arg or Lys
QLQLQESGPGLVKPSETLSLTCTVSGFSLTNYGLHWIRQPPGKGLEWIGVIWSGGKTDYNPSLKSRVTISXDTSKNQFSLKLSSVXAXDTAVYYCAXYDYDDSLDYWGQGTLVTVSS

SEQ ID NO: 50 - protein, 10 aa, Artificial Sequence
  VH CDR3
LWSRGGYFDY

SEQ ID NO: 51 - protein, 247 aa, Artificial Sequence
  AB0191 (VH-VL)
EVQLVESGGGVVQPGGSLRLSCAASGFTFNSFGMHWVRQAPGKCLEWVAFISSGSTSIYYANTVKGRFTISRDNSKNTLYLQMNSLRAEDTAVYYCARDGYPTGGAMDYWGQGTSVTVSSGGGGSGGGGSGGGGSGGGGSDIQMTQSPSSLSASVGDRVTITCKASQDIYNYLSWFQQKPGKAPKPLIYRANILVSGVPSRFSGSGSGQDYTFTISSLQPEDIATYYCLQFDAFPFTFGCGTKLEIK

SEQ ID NO: 52 - protein, 247 aa, Artificial Sequence
  AB0185 (VL-VH)
DIQMTQSPSSLSASVGDRVTITCKASQDIYNYLSWFQQKPGKAPKPLIYRANILVSGVPSRFSGSGSGQDYTFTISSLQPEDIATYYCLQFDAFPFTFGCGTKLEIKGGGGSGGGGSGGGGSGGGGSEVQLVESGGGVVQPGGSLRLSCAASGFTFNSFGMHWVRQAPGKCLEWVAFISSGSTSIYYANTVKGRFTISRDNSKNTLYLQMNSLRAEDTAVYYCARDGYPTGGAMDYWGQGTSVTVSS

SEQ ID NO: 53 - protein, 10 aa, Artificial Sequence
  VL CDR1
SARSSVSYMS

SEQ ID NO: 54 - protein, 11 aa, Artificial Sequence
  VH CDR3
DGYPTGGAMDY

SEQ ID NO: 55 - protein, 7 aa, Artificial Sequence
  VL CDR2
GISKLAS

SEQ ID NO: 56 - protein, 9 aa, Artificial Sequence
  VL CDR3
QQRSYYPFT

SEQ ID NO: 57 - protein, 120 aa, Artificial Sequence
  13E1.A4 VH
EVQLQESGPELEKPGASVRISCKASGYSFTAYNMNWVKQSNGKSLEWIGNIDPSYGDATYNQKFKGKATLTVDKSSSTAYMQLKSLTSEDSAVYYCARDNYYGSGYFDYWGQGTTLTVSS

SEQ ID NO: 58 - protein, 112 aa, Artificial Sequence
  13E1.A4 VL
SVLMTQTPLSLPVSLGDRASISCRSSQGIVHINGNTYLEWYLQKPGQSPKLLIYKVSNRFSGVPDRFSGSGSGTDFTLKISRVEAEDLGVYYCFQGSHVPWTFGGGTKLEIK

SEQ ID NO: 59 - protein, 7 aa, Artificial Sequence
  VH CDR1
GFTFNSF

SEQ ID NO: 60 - protein, 120 aa, Artificial Sequence
  9F11.B7 VH
EVQLQESGGGLVQPGGSRKLSCAASGFTFNSFGMHWVRQAPEKGLEWVAFISSGSTSIYYANTVKGRFTISRDNPKNTLFLQMTSLRSEDTAMYYCARDGYPTGGAMDYWGQGTSVTVSS

SEQ ID NO: 61 - protein, 107 aa, Artificial Sequence
  9F11.B7 VL
DIKMTQSPSSMYASLGERVTITCKASQDIYNYLSWFQLKPGKSPRPLIYRANILVSGVPSKFSGSGSGQDYSLTINSLEYEDLGIYYCLQFDAFPFTFGSGTKLEIK

SEQ ID NO: 62 - protein, 7 aa, Artificial Sequence
  VH CDR1
GFTFNAF

SEQ ID NO: 63 - protein, 6 aa, Artificial Sequence
  VH CDR2
SSGSTS

SEQ ID NO: 64 - protein, 7 aa, Artificial Sequence
  VH CDR1
GYSFTAY

SEQ ID NO: 65 - protein, 11 aa, Artificial Sequence
  VL CDR1
KASQDIYNYLS

SEQ ID NO: 66 - protein, 7 aa, Artificial Sequence
  VL CDR2
RANILVS

SEQ ID NO: 67 - protein, 9 aa, Artificial Sequence
  VL CDR3
LQFDAFPFT

SEQ ID NO: 68 - protein, 6 aa, Artificial Sequence
  VH CDR2
DPSYGD

SEQ ID NO: 69 - protein, 107 aa, Artificial Sequence
  Humanized 9F11.B7 in AB0191/AB0185 VL
DIQMTQSPSSLSASVGDRVTITCKASQDIYNYLSWFQQKPGKAPKPLIYRANILVSGVPSRFSGSGSGQDYTFTISSLQPEDIATYYCLQFDAFPFTFGSGTKLEIK

SEQ ID NO: 70 - protein, 247 aa, Artificial Sequence
  AB0192 (VH-VL)
EVQLVESGGGVVQPGGSLRLSCAASGFTFNAFGMHWVRQAPGKCLEWVAFISSGSTSIYYANTVKGRFTISRDNSKNTLYLQMNSLRAEDTAVYYCARDGYPTGGAMDYWGQGTSVTVSSGGGGSGGGGSGGGGSGGGGSDIQMTQSPSSLSASVGDRVTITCKASQDIYNYLSWFQQKPGKAPKPLIYRANILVSGVPSRFSGSGSGQDYTFTISSLQPEDIATYYCLQFDAFPFTFGCGTKLEIK

SEQ ID NO: 71 - protein, 247 aa, Artificial Sequence
  AB0186 (VL-VH)
DIQMTQSPSSLSASVGDRVTITCKASQDIYNYLSWFQQKPGKAPKPLIYRANILVSGVPSRFSGSGSGQDYTFTISSLQPEDIATYYCLQFDAFPFTFGCGTKLEIKGGGGSGGGGSGGGGSGGGGSEVQLVESGGGVVQPGGSLRLSCAASGFTFNAFGMHWVRQAPGKCLEWVAFISSGSTSIYYANTVKGRFTISRDNSKNTLYLQMNSLRAEDTAVYYCARDGYPTGGAMDYWGQGTSVTVSS

SEQ ID NO: 72 - protein, 7 aa, Artificial Sequence
  VH CDR1
GFSLTSY

SEQ ID NO: 73 - protein, 11 aa, Artificial Sequence
  VH CDR3
DNYYGSGYFDY

SEQ ID NO: 74 - protein, 247 aa, Artificial Sequence
  AB0193 (VH-VL)
EVQLVESGGGVVQPGGSLRLSCAASGFTFNSFGMHWVRQAPGKCLEWVAFISSGSTSIYYANTVKGRFTISRDNSKNTLYLQMNSLRAEDTAVYYCARSGYPTGGAMDYWGQGTSVTVSSGGGGSGGGGSGGGGSGGGGSDIQMTQSPSSLSASVGDRVTITCKASQDIYNYLSWFQQKPGKAPKPLIYRANILVSGVPSRFSGSGSGQDYTFTISSLQPEDIATYYCLQFDAFPFTFGCGTKLEIK

SEQ ID NO: 75 - protein, 247 aa, Artificial Sequence
  AB0187 (VL-VH)
DIQMTQSPSSLSASVGDRVTITCKASQDIYNYLSWFQQKPGKAPKPLIYRANILVSGVPSRFSGSGSGQDYTFTISSLQPEDIATYYCLQFDAFPFTFGCGTKLEIKGGGGSGGGGSGGGGSGGGGSEVQLVESGGGVVQPGGSLRLSCAASGFTFNSFGMHWVRQAPGKCLEWVAFISSGSTSIYYANTVKGRFTISRDNSKNTLYLQMNSLRAEDTAVYYCARSGYPTGGAMDYWGQGTSVTVSS

SEQ ID NO: 76 - protein, 120 aa, Artificial Sequence
  Humanized 9F11.B7 in AB0193/AB0187 VH
EVQLVESGGGVVQPGGSLRLSCAASGFTFNSFGMHWVRQAPGKGLEWVAFISSGSTSIYYANTVKGRFTISRDNSKNTLYLQMNSLRAEDTAVYYCARSGYPTGGAMDYWGQGTSVTVSS

SEQ ID NO: 77 - protein, 16 aa, Artificial Sequence
  VL CDR1
RSSQGIVHINGNTYLE

SEQ ID NO: 78 - protein, 7 aa, Artificial Sequence
  VL CDR2
KVSNRFS

SEQ ID NO: 79 - protein, 11 aa, Artificial Sequence
  VH CDR3
SGYPTGGAMDY

SEQ ID NO: 80 - protein, 9 aa, Artificial Sequence
  VL CDR3
FQGSHVPWT

SEQ ID NO: 81 - protein, 247 aa, Artificial Sequence
  AB0194 (VH-VL)
EVQLVESGGGVVQPGGSLRLSCAASGFTFNSFGMHWVRQAPGKCLEWVAFISSGSTSIYYANTVKGRFTISRDNSKNTLYLQMNSLRAEDTAVYYCARDGYPTGGAMDYWGQGTSVTVSSGGGGSGGGGSGGGGSGGGGSDIKMTQSPSSLSASVGDRVTITCKASQDIYNYLSWFQQKPGKAPKPLIYRANILVSGVPSRFSGSGSGQDYTLTISSLQPEDIATYYCLQFDAFPFTFGCGTKLEIK

SEQ ID NO: 82 - protein, 247 aa, Artificial Sequence
  AB0188 (VL-VH)
DIKMTQSPSSLSASVGDRVTITCKASQDIYNYLSWFQQKPGKAPKPLIYRANILVSGVPSRFSGSGSGQDYTLTISSLQPEDIATYYCLQFDAFPFTFGCGTKLEIKGGGGSGGGGSGGGGSGGGGSEVQLVESGGGVVQPGGSLRLSCAASGFTFNSFGMHWVRQAPGKCLEWVAFISSGSTSIYYANTVKGRFTISRDNSKNTLYLQMNSLRAEDTAVYYCARDGYPTGGAMDYWGQGTSVTVSS

SEQ ID NO: 83 - protein, 121 aa, Artificial Sequence
  12F8.H7 VH
EVQLQESGAELVRSGASVKLSCTVSGFNIKDYYMHWVKQRPEQGLEWIGWIDPENGDTENVPKFQGKATMTADTSSNTAYLQLRSLTSEDTAVYYCKSYYYDSSSRYVDVWGAGTTVTVSS

SEQ ID NO: 84 - protein, 107 aa, Artificial Sequence
  Humanized 9F11.B7 in AB0194/AB0188 VL
DIKMTQSPSSLSASVGDRVTITCKASQDIYNYLSWFQQKPGKAPKPLIYRANILVSGVPSRFSGSGSGQDYTLTISSLQPEDIATYYCLQFDAFPFTFGSGTKLEIK

SEQ ID NO: 85 - protein, 112 aa, Artificial Sequence
  12F8.H7 VL
GIVMTQAPLTLSVTIGQPASISCKSSQSLLDSDGKTFLNWFLQRPGQSPKRLISLVSKLDSGVPDRFTGSGSGTDFTLKLSRVEPEDLGVYYCWQGTHFPYTFGGGTKLEIK

SEQ ID NO: 86 - protein, 7 aa, Artificial Sequence
  VH CDR1
GFNIKDY

SEQ ID NO: 87 - protein, 7 aa, Artificial Sequence
  VH CDR1
GFSLTSF

SEQ ID NO: 88 - protein, 6 aa, Artificial Sequence
  VH CDR2
DPENGD

SEQ ID NO: 89 - protein, 7 aa, Artificial Sequence
  VH CDR3
SYFAMDY

SEQ ID NO: 90 - protein, 16 aa, Artificial Sequence
  VL CDR1
KSSQSLLDSDGKTFLN

SEQ ID NO: 91 - protein, 7 aa, Artificial Sequence
  VL CDR2
LVSKLDS

SEQ ID NO: 92 - protein, 7 aa, Artificial Sequence
  VL CDR2
GASIRES

SEQ ID NO: 93 - protein, 9 aa, Artificial Sequence
  VL CDR3
WQGTHFPYT

SEQ ID NO: 94 - protein, 119 aa, Artificial Sequence
  9E4.B7 VH
EVQLQESGAELMKPGASVKISCRTTGYTFSTYWIEWVKQRPGRGPEWIGELFPGNSDTTLNEKFTGKATFTADSSSNTAYMQLSSLTSEDSAVYYCARSGYYGSSLDYWGQGTTLTVSS

SEQ ID NO: 95 - protein, 107 aa, Artificial Sequence
  9E4.B7 VL
GIVMTQSPASLSASVGETVTITCRAGENIHSYLAWYQQKQGKSPQLLVYNAKTLAEGVPSRFSGSGSGTQFSLKINSLQPEDFGSYYCQHHYGTPRTFGGGTKLEIK

SEQ ID NO: 96 - protein, 7 aa, Artificial Sequence
  VH CDR1
GYTFSTY

SEQ ID NO: 97 - protein, 6 aa, Artificial Sequence
  VH CDR2
FPGNSD

SEQ ID NO: 98 - protein, 10 aa, Artificial Sequence
  VH CDR3
SGYYGSSLDY

SEQ ID NO: 99 - protein, 11 aa, Artificial Sequence
  VL CDR1
RAGENIHSYLA

SEQ ID NO: 100 - protein, 7 aa, Artificial Sequence
  VL CDR2
NAKTLAE

SEQ ID NO: 101 - protein, 9 aa, Artificial Sequence
  VL CDR3
QHHYGTPRT

SEQ ID NO: 102 - protein, 5 aa, Artificial Sequence
  VH CDR2
WSGGN

SEQ ID NO: 103 - protein, 115 aa, Artificial Sequence
  20D6.H8 VL
GIVMTQSPSSLAVTAGEKVTMRCKSSQSLLWNVNQNNYLVWYQQKQGQPPKLLIYGASIRESWVPDRFTGSGSGTDFTLTISNVHAEDLAVYYCQHNHGSFLPYTFGGGTKLEIK

SEQ ID NO: 104 - protein, 115 aa, Artificial Sequence
  20D6.H8 VH
EVQLQESGPGLVQPSQSLSITCTVSGFSLTSFGIHWVRQSPGKGLEWLGVIWSGGNTDSNAAFISRLSITKDISKSQVFFKMNSLQVTDTAIYYCARSYFAMDYWGQGTSVTVSS

SEQ ID NO: 105 - protein, 7 aa, Artificial Sequence
  VL CDR2
GASIRQS

SEQ ID NO: 106 - protein, 17 aa, Artificial Sequence
  VL CDR1
KSSQSLLWNVNQNNYLL

SEQ ID NO: 107 - protein, 7 aa, Artificial Sequence
  VH CDR3
THFGMDY

SEQ ID NO: 108 - protein, 115 aa, Artificial Sequence
  23A5.H8 VH
QVQLRQSGPGLVQPSQSLSITCTVSGFSLTSYGVHWVRQSPGKGLEWLGVMWSGGSTDYNAAFMSRLSISKDNSKSQVFFTMNSLQADDTAIYYCARTHFGMDYWGQGTPVTVSS

SEQ ID NO: 109 - protein, 115 aa, Artificial Sequence
  23A5.H8 VL
GIVMTQSPSSLAVTAGEKVTMRCKSSQSLLWSVNQNNYLLWYQQKQGQPPKLLIYGASIRQSWVPDRFTGSGSGTDFTLSISNVHAEDLAVYYCQHNHGSFLPYTFGGGTKLEIK

SEQ ID NO: 110 - protein, 117 aa, Artificial Sequence
  Humanized 16B8.C8 in scFv-1294/scFv-1303 VH
QLQLQESGPGLVKPSETLSLTCTVSGFSLTNYGLHWIRQPPGKGLEWIGVIWSGGKTDYNPSLKSRVTISKDTSKNQFSLKLSSVTANDTAVYYCAKYDYDDSLDYWGQGTLVTVSS

SEQ ID NO: 111 - protein, 17 aa, Artificial Sequence
  VL CDR1
KSSQSLLWSVNQNNYLL

SEQ ID NO: 112 - protein, 11 aa, Artificial Sequence
  Humanized 9F11.B7 VH CDR3
  misc_feature (1)..(1): Xaa can be Asp or Ser
XGYPTGGAMDY

SEQ ID NO: 113 - protein, 115 aa, Artificial Sequence
  30A9.E9 VH
EVQLQESGPGLVQPSQSLSITCTVSGFSLTSFGVHWVRQSPGKGLEWLGVIWSGGSTDSNAAFISRLTITKDNSKSQVFFKMNSLQATDTAIYYCARSYFAMDYWGQGTSVSVSS

SEQ ID NO: 114 - protein, 115 aa, Artificial Sequence
  30A9.E9 VL
DIVMTQSPSSLAVTAGEKVTMRCKSSQSLLWNVNQNNYLLWYQQKQGQPPKLLIYGASIRESWVPDRFTGSGSGTDFTLTISNVHVEDLAVYYCQHNHGSFLPYTFGGGTKLEIK

SEQ ID NO: 115 - protein, 120 aa, Artificial Sequence
  Humanized 9F11.B7 VH
  misc_feature (31)..(31): Xaa can be Ser or Ala
  misc_feature (99)..(99): Xaa can be Asp or Ser
EVQLVESGGGVVQPGGSLRLSCAASGFTFNXFGMHWVRQAPGKGLEWVAFISSGSTSIYYANTVKGRFTISRDNSKNTLYLQMNSLRAEDTAVYYCARXGYPTGGAMDYWGQGTSVTVSS

SEQ ID NO: 116 - protein, 107 aa, Artificial Sequence
  Humanized 9F11.B7 VL
  misc_feature (3)..(3): Xaa can be Gln or Lys
  misc_feature (73)..(73): Xaa can be Phe or Leu
DIXMTQSPSSLSASVGDRVTITCKASQDIYNYLSWFQQKPGKAPKPLIYRANILVSGVPSRFSGSGSGQDYTXTISSLQPEDIATYYCLQFDAFPFTFGSGTKLEIK

SEQ ID NO: 117 - protein, 7 aa, Artificial Sequence
  VH CDR1
  misc_feature (6)..(6): Xaa can be Ser or Ala
GFTFNXF

SEQ ID NO: 118 - protein, 247 aa, Artificial Sequence
  AB0195 (VH-VL)
EVQLVESGGGVVQPGGSLRLSCAASGFTFNAFGMHWVRQAPGKCLEWVAFISSGSTSIYYANTVKGRFTISRDNSKNTLYLQMNSLRAEDTAVYYCARDGYPTGGAMDYWGQGTSVTVSSGGGGSGGGGSGGGGSGGGGSDIKMTQSPSSLSASVGDRVTITCKASQDIYNYLSWFQQKPGKAPKPLIYRANILVSGVPSRFSGSGSGQDYTLTISSLQPEDIATYYCLQFDAFPFTFGCGTKLEIK

SEQ ID NO: 119 - protein, 247 aa, Artificial Sequence
  AB0189 (VL-VH)
DIKMTQSPSSLSASVGDRVTITCKASQDIYNYLSWFQQKPGKAPKPLIYRANILVSGVPSRFSGSGSGQDYTLTISSLQPEDIATYYCLQFDAFPFTFGCGTKLEIKGGGGSGGGGSGGGGSGGGGSEVQLVESGGGVVQPGGSLRLSCAASGFTFNAFGMHWVRQAPGKCLEWVAFISSGSTSIYYANTVKGRFTISRDNSKNTLYLQMNSLRAEDTAVYYCARDGYPTGGAMDYWGQGTSVTVSS

SEQ ID NO: 120 - protein, 247 aa, Artificial Sequence
  AB0196 (VH-VL)
EVQLVESGGGVVQPGGSLRLSCAASGFTFNSFGMHWVRQAPGKCLEWVAFISSGSTSIYYANTVKGRFTISRDNSKNTLYLQMNSLRAEDTAVYYCARSGYPTGGAMDYWGQGTSVTVSSGGGGSGGGGSGGGGSGGGGSDIKMTQSPSSLSASVGDRVTITCKASQDIYNYLSWFQQKPGKAPKPLIYRANILVSGVPSRFSGSGSGQDYTLTISSLQPEDIATYYCLQFDAFPFTFGCGTKLEIK

SEQ ID NO: 121 - protein, 247 aa, Artificial Sequence
  AB0190 (VL-VH)
DIKMTQSPSSLSASVGDRVTITCKASQDIYNYLSWFQQKPGKAPKPLIYRANILVSGVPSRFSGSGSGQDYTLTISSLQPEDIATYYCLQFDAFPFTFGCGTKLEIKGGGGSGGGGSGGGGSGGGGSEVQLVESGGGVVQPGGSLRLSCAASGFTFNSFGMHWVRQAPGKCLEWVAFISSGSTSIYYANTVKGRFTISRDNSKNTLYLQMNSLRAEDTAVYYCARSGYPTGGAMDYWGQGTSVTVSS

SEQ ID NO: 122 - protein, 117 aa, Artificial Sequence
  Humanized 16B8.C8 in scFv-1296/scFv-1305 VH
QLQLQESGPGLVKPSETLSLTCTVSGFSLTNYGLHWIRQPPGKGLEWIGVIWSGGKTDYNPSLKSRVTISKDTSKNQFSLKLSSVQANDTAVYYCARYDYDDSLDYWGQGTLVTVSS

SEQ ID NO: 123 - protein, 118 aa, Artificial Sequence
  Anti-CLEC12A mAbs Merus-CLL1 - VH
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGNYGDEFDYWGQGTLVTVSS

SEQ ID NO: 124 - protein, 107 aa, Artificial Sequence
  Anti-CLEC12A mAbs Merus-CLL1 - VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 125 - protein, 111 aa, Artificial Sequence
  Anti-CLEC12A mAbs Genentech-h6E7 - VH
DIQMTQSPSSLSASVGDRVTITCRASQSVSTSSYNYMHWYQQKPGKPPKLLIKYASNLESGVPSRFSGSGSGTDFTLTISSLQPEDFATYYCQHSWEIPLTFGQGTKVEIK

SEQ ID NO: 126 - protein, 122 aa, Artificial Sequence
  Anti-CLEC12A mAbs Genentech-h6E7 - VL
EVQLVQSGAEVKKPGASVKVSCKASGYSFTDYYMHWVRQAPGQGLEWIGRINPYNGAAFYSQNFKDRVTLTVDTSTSTAYLELSSLRSEDTAVYYCAIERGADLEGYAMDYWGQGTLVTVSS

SEQ ID NO: 127 - protein, 12 aa, Artificial Sequence
  VH CDR3
YYYDSSSRYVDV

SEQ ID NO: 128 - protein, 117 aa, Artificial Sequence
  Humanized 16B8.C8 in AB0305/AB5030 VH
QLQLQESGPGLVKPSETLSLTCTVSGFSLTNYGLHWIRQPPGKGLEWIGVIWVGGATDYNPSLKSRVTISVDTSKNQFSLKLSSVQAADTAVYYCAKGDYGDTLDYWGQGTLVTVSS

SEQ ID NO: 129 - protein, 107 aa, Artificial Sequence
  Humanized 16B8.C8 in AB0305/AB5030 VL
DIQMTQSPSSLSASVGDRVTITCHASQNINFWLSWYQQKPGKAPKLLIYEASNLHTGVPSRFSGSGSGTDFTLTISSLQPEDFATYYCQQSHSYPLTFGSGTKLEIK

SEQ ID NO: 130 - protein, 5 aa, Artificial Sequence
  VH CDR2
WVGGA

SEQ ID NO: 131 - protein, 9 aa, Artificial Sequence
  VH CDR3
GDYGDTLDY

SEQ ID NO: 132 - protein, 244 aa, Artificial Sequence
  AB0305 (VH-VL)
QLQLQESGPGLVKPSETLSLTCTVSGFSLTNYGLHWIRQPPGKCLEWIGVIWVGGATDYNPSLKSRVTISVDTSKNQFSLKLSSVQAADTAVYYCAKGDYGDTLDYWGQGTLVTVSSGGGGSGGGGSGGGGSGGGGSDIQMTQSPSSLSASVGDRVTITCHASQNINFWLSWYQQKPGKAPKLLIYEASNLHTGVPSRFSGSGSGTDFTLTISSLQPEDFATYYCQQSHSYPLTFGCGTKLEIK

SEQ ID NO: 133 - protein, 244 aa, Artificial Sequence
  AB5030 (VL-VH)
DIQMTQSPSSLSASVGDRVTITCHASQNINFWLSWYQQKPGKAPKLLIYEASNLHTGVPSRFSGSGSGTDFTLTISSLQPEDFATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVIWVGGATDYNPSLKSRVTISVDTSKNQFSLKLSSVQAADTAVYYCAKGDYGDTLDYWGQGTLVTVSS

SEQ ID NO: 134 - protein, 117 aa, Artificial Sequence
  Humanized 16B8.C8 in AB0147/AB7410 VH
QLQLQESGPGLVKPSETLSLTCTVSGFSLTNYGLHWIRQPPGKGLEWIGVILSGGWTDYNPSLKSRVTISKDTSKNQFSLKLSSVQAADTAVYYCAKGDYGDALDYWGQGTLVTVSS

SEQ ID NO: 135 - protein, 107 aa, Artificial Sequence
  Humanized 16B8.C8 in AB0147/AB7410 VL
DIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGSGTKLEIK

SEQ ID NO: 136 - protein, 5 aa, Artificial Sequence
  VH CDR2
LSGGW

SEQ ID NO: 137 - protein, 9 aa, Artificial Sequence
  VH CDR3
GDYGDALDY

SEQ ID NO: 138 - protein, 244 aa, Artificial Sequence
  AB0037 (VH-VL)
QLQLQESGPGLVKPSETLSLTCTVSGFSLTNYGLHWIRQPPGKCLEWIGVILSGGWTDYNPSLKSRVTISKDTSKNQFSLKLSSVQAADTAVYYCAKGDYGDALDYWGQGTLVTVSSGGGGSGGGGSGGGGSGGGGSDIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGCGTKLEIK

SEQ ID NO: 139 - protein, 244 aa, Artificial Sequence
  AB7300 (VL-VH)
DIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGCGTKLEIKGGGGSGGGGSGGGGSGGGGSQLQLQESGPGLVKPSETLSLTCTVSGFSLTNYGLHWIRQPPGKCLEWIGVILSGGWTDYNPSLKSRVTISKDTSKNQFSLKLSSVQAADTAVYYCAKGDYGDALDYWGQGTLVTVSS

SEQ ID NO: 140 - protein, 107 aa, Artificial Sequence
  Humanized 16B8.C8 in scFv-1602/scFv-2061 VL
DIQMTQSPSSLSASVGDRVTITCHASQNINFWLSWYQQKPGKAPKLLIYEASNLHTGVPSRFSGSGSGTRFTLTISSLQPEDIATYYCQQSHSYPLTFGGGTKLEIK

SEQ ID NO: 141 - protein, 9 aa, Artificial Sequence
  Variant used to identify alternative sequence motifs without the DS site
YDYDDALDY

SEQ ID NO: 142 - protein, 9 aa, Artificial Sequence
  Variant used to identify alternative sequence motifs without the DS site
YDYDDILDY

SEQ ID NO: 143 - protein, 9 aa, Artificial Sequence
  variant used to identify alternative sequence motifs without the DS site
YDYDDLLDY

SEQ ID NO: 144 - protein, 9 aa, Artificial Sequence
  variant of binder to hCLEC12A
YDYDDVLDY

SEQ ID NO: 145 - protein, 9 aa, Artificial Sequence
  variant of non-binder to hCLEC12A
YDYDDTLDY

SEQ ID NO: 146 - protein, 9 aa, Artificial Sequence
  variant of non-binder to hCLEC12A
YDYDESLDY

SEQ ID NO: 147 - protein, 117 aa, Artificial Sequence
  CLEC12A VH
QLQLQESGPGLVKPSETLSLTCTVSGFSLTNYGLHWIRQPPGKCLEWIGVIWVGGATDYNPSLKSRVTISVDTSKNQFSLKLSSVQAADTAVYYCAKGDYGDTLDYWGQGTLVTVSS

SEQ ID NO: 148 - protein, 107 aa, Artificial Sequence
  CLEC12A VL
DIQMTQSPSSLSASVGDRVTITCHASQNINFWLSWYQQKPGKAPKLLIYEASNLHTGVPSRFSGSGSGTDFTLTISSLQPEDFATYYCQQSHSYPLTFGCGTKLEIK

(19) United States
(12) Patent Application Publication
(10) Pub. No.: US 2023/0220084 A1
(43) Pub. Date: Jul. 13, 2023

(54) Antibodies Targeting CLEC12A and Use Thereof

(71) Applicant: Dragonfly Therapeutics, Inc., Waltham, MA (US)

(72) Inventors: Hemanta Baruah, Euless, TX (US); Gregory P. Chang, Medford, MA (US); Ann F. Cheung, Lincoln, MA (US); Asya Grinberg, Lexington, MA (US)

(21) Appl. No.: 17/923,411

(22) PCT Filed: May 5, 2021
(86) PCT No.: PCT/US2021/030773

§ 371 Date: Nov. 4, 2022

Related U.S. Application Data
(60) Provisional application No. 63/020,806, filed on May 6, 2020.

(51) Int. Cl. C07K 16/28; C07K 14/725; A61P 35/00; C12N 15/63

(52) U.S. Cl. CPC C07K 16/2851; C07K 14/7051; A61P 35/00; C12N 15/63; C07K 2317/565; C07K 2317/92; C07K 2317/622

(57) ABSTRACT

Disclosed are proteins with antibody heavy chain and light chain variable domains that can be paired to form an antigen-binding site targeting CLEC12A on a cell, pharmaceutical compositions comprising such proteins, and therapeutic methods using such proteins and pharmaceutical compositions, including for the treatment of cancer.

This application claims priority to U.S. Provisional Application No. 63/020,806, filed on May 6, 2020, the entirety of which is incorporated herein by reference.

### SEQUENCE LISTING

This application incorporates by reference in its entirety the Computer Readable Form (CRF) of a Sequence Listing in ASCII text format. The Sequence Listing text file is entitled “14247-539-228_SEQ_LISTING,” was created on May 3, 2021, and is 147,674 bytes in size.

### FIELD OF THE INVENTION

The present application provides proteins with antibody heavy chain and light chain variable domains that can be paired to form an antigen-binding site targeting CLL-1/CLEC12A on a cell, pharmaceutical compositions comprising such proteins, and therapeutic methods using such proteins and pharmaceutical compositions, including for the treatment of cancer.

### BACKGROUND

Cancer continues to be a significant health problem despite the substantial research efforts and scientific advances reported in the literature for treating this disease. Some of the most frequently diagnosed cancers in adults include prostate cancer, breast cancer, and lung cancer. Hematological malignancies, though less frequent than solid cancers, have low survival rates. Current treatment options for these cancers are not effective for all patients and/or can have substantial adverse side effects. Other types of cancer also remain challenging to treat using existing therapeutic options.

C-type lectin domain family 12 member A (CLEC12A), also known as C-type lectin-like molecule-1 (CLL-1) or myeloid inhibitory C-type lectin-like receptor (MICL), is a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. CLEC12A, a type II transmembrane glycoprotein, is overexpressed in over 90% of acute myeloid leukemia patient on leukemic stem cells, but not on normal haematopoietic cells.

Despite many efforts undertaken by several biotech and pharma companies, development of specific CLEC12A targeted biologics is hindered by the absence of antibodies with good developability characteristics. The challenges in discovering CLEC12A antibodies may be attributed to the complexity of the antigen. CLEC12A is a monomeric heavily glycosylated protein with six potential N-glycosylation sites within an extracellular domain having 201 amino acids. Four out of six N-glycosylation sites are clustered in the membrane proximal domain of the molecule and are likely to be involved in presentation of the target on the cell surface. Variations in the glycosylation status of CLEC12A on the surface of different cell types has been reported (Marshall et al, (2006) Eur J Immunol. 36(8):2159-69). Therefore, there still remains a need in the field for new and useful antibodies that bind CLEC12A, particularly antibodies that bind to CLEC12A in a glycosylation independent manner.

### SUMMARY OF THE APPLICATION

The present application provides antigen-binding sites that bind human CLEC12A. These antigen-binding sites bind various epitopes in an extracellular domain of CLEC12A, and some of them bind CLEC12A in a glycosylation independent manner. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating CLEC12A-associated diseases such as cancer.

Accordingly, in one aspect, the present application provides an antigen-binding site that binds CLEC12A comprising:

(a) a heavy chain variable domain (VH) comprising complementarity-determining region 1 (CDR1), complementarity-determining region 2 (CDR2), and complementarity-determining region 3 (CDR3) comprising the amino acid sequences of SEQ ID NOs: 11, 4, and 5, respectively; and

(b) a light chain variable domain (VL) comprising CDR1, CDR2, and CDR3 comprising the amino acid sequences of SEQ ID NOs: 6, 7, and 8, respectively.

In some embodiments, the VH comprises an amino acid sequence of SEQ ID NO:49, and the VL comprises an amino acid sequence of SEQ ID NO:17. In certain embodiments, the VH comprises an amino acid sequence at least 90% identical to SEQ ID NO:45, and the VL comprises an amino acid sequence at least 90% identical to SEQ ID NO:140. In certain embodiments, the VH comprises the amino acid sequence of SEQ ID NO:45, and the VL comprises the amino acid sequence of SEQ ID NO:140. In some embodiments, the VH and the VL comprise the amino acid sequences of SEQ ID NOs: 9 and 10; 13 and 10; 110 and 10; 45 and 10; 122 and 10; 9 and 30; 9 and 34; 9 and 38; or 41 and 42, respectively.

In another aspect, the present application provides an antigen-binding site that binds CLEC12A, comprising:

(a) a VH comprising CDR1, CDR2, and CDR3 comprising the amino acid sequences of SEQ ID NOs: 117, 63, and 112, respectively; and

(b) a VL comprising CDR1, CDR2, and CDR3 comprising the amino acid sequences of SEQ ID NOs: 65, 66, and 67, respectively.

In some embodiments, the VH comprises CDR1, CDR2, and CDR3 of SEQ ID NO: 59, 63, and 79, respectively; and the VL comprises CDR1, CDR2, and CDR3 of SEQ ID NO: 65, 66, and 67, respectively. In some embodiments, the VH comprises CDR1, CDR2, and CDR3 of SEQ ID NO: 59, 63, and 54, respectively, and the VL comprises CDR1, CDR2, and CDR3 of SEQ ID NO: 65, 66, and 67, respectively. In some embodiments, the VH comprises CDR1, CDR2, and CDR3 of SEQ ID NO: 62, 63, and 54, respectively, and the VL comprises CDR1, CDR2, and CDR3 of SEQ ID NO: 65, 66, and 67, respectively. In certain embodiments, the VH comprises the amino acid sequence of SEQ ID NO:115, and the VL comprises the amino acid sequence of SEQ ID NO:116. In certain embodiments, the VH and the VL comprise the amino acid sequences of SEQ ID NOs: 29 and 69; 14 and 69; 76 and 69; 29 and 84; 14 and 84; or 76 and 84, respectively.

In another aspect, the present application provides an antigen-binding site that binds CLEC12A, comprising:

(a) a VH comprising CDR1, CDR2, and CDR3 comprising the amino acid sequences of SEQ ID NOs: 87, 33, and 89, respectively; and

(b) a VL comprising CDR1, CDR2, and CDR3 comprising the amino acid sequences of SEQ ID NOs: 106, 92, and 46, respectively.

In another aspect, the present application provides an antigen-binding site that binds CLEC12A, comprising:

(a) a VH comprising CDR1, CDR2, and CDR3 comprising the amino acid sequences of SEQ ID NOs: 72, 33, and 107, respectively; and

(b) a VL comprising CDR1, CDR2, and CDR3 comprising the amino acid sequences of SEQ ID NOs: 111, 105, and 46, respectively.

In another aspect, the present application provides an antigen-binding site that binds CLEC12A, comprising:

(a) a VH comprising CDR1, CDR2, and CDR3 comprising the amino acid sequences of SEQ ID NOs: 87, 102, and 89, respectively; and

(b) a VL comprising CDR1, CDR2, and CDR3 comprising the amino acid sequences of SEQ ID NOs: 18, 92, and 46, respectively.

In another aspect, the present application provides an antigen-binding site that binds CLEC12A, comprising:

(a) a VH comprising CDR1, CDR2, and CDR3 comprising the amino acid sequences of SEQ ID NOs: 26, 37, and 50, respectively; and

(b) a VL comprising CDR1, CDR2, and CDR3 comprising the amino acid sequences of SEQ ID NOs: 53, 55, and 56, respectively.

In another aspect, the present application provides an antigen-binding site that binds CLEC12A, comprising:

(a) a VH comprising CDR1, CDR2, and CDR3 comprising the amino acid sequences of SEQ ID NOs: 64, 68, and 73, respectively; and

(b) a VL comprising CDR1, CDR2, and CDR3 comprising the amino acid sequences of SEQ ID NOs: 77, 78, and 80, respectively.

In another aspect, the present application provides an antigen-binding site that binds CLEC12A, comprising:

(a) a VH comprising CDR1, CDR2, and CDR3 comprising the amino acid sequences of SEQ ID NOs: 86, 88, and 127, respectively; and

(b) a VL comprising CDR1, CDR2, and CDR3 comprising the amino acid sequences of SEQ ID NOs: 90, 91, and 93, respectively.

In another aspect, the present application provides an antigen-binding site that binds CLEC12A, comprising:

(a) a VH comprising CDR1, CDR2, and CDR3 comprising the amino acid sequences of SEQ ID NOs: 96, 97, and 98, respectively; and

(b) a VL comprising CDR1, CDR2, and CDR3 comprising the amino acid sequences of SEQ ID NOs: 99, 100, and 101, respectively.

In another aspect, the present application provides an antigen-binding site that competes with the antigen-binding site disclosed above.

In some embodiments of the foregoing aspects, the antigen-binding site binds human CLEC12A with a dissociation constant (KD) smaller than or equal to 20 nM as measured by surface plasmon resonance (SPR). In certain embodiments, the antigen-binding site binds human CLEC12A with a KD smaller than or equal to 1 nM as measured by SPR. In some embodiments, the antigen-binding site binds CLEC12A in a glycosylation independent manner. In some embodiments, the antigen-binding site binds human CLEC12A comprising a K244Q mutation.

In some embodiments, the antigen-binding site is present as a single-chain fragment variable (scFv). In certain embodiments, the scFv comprises an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, 82, 118, 119, 120, 121, 132, 133, 138, and 139.

In another aspect, the present application provides an antigen-binding site that binds CLEC12A in a glycosylation independent manner.

In another aspect, the present application provides a protein comprising an antigen-binding site disclosed herein. In some embodiments, the proteins further comprise an antibody heavy chain constant region. In certain embodiments, the antibody heavy chain constant region is a human IgG heavy chain constant region. In certain embodiments, the antibody heavy chain constant region is a human IgG1 heavy chain constant region. In certain embodiments, each polypeptide chain of the antibody heavy chain constant region comprises an amino acid sequence at least 90% identical to SEQ ID NO:21.

In certain embodiments, at least one polypeptide chain of the antibody heavy chain constant region comprises one or more mutations, relative to SEQ ID NO:21, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system. In certain embodiments, at least one polypeptide chain of the antibody heavy chain constant region comprises one or more mutations, relative to SEQ ID NO:21, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

In certain embodiments, one polypeptide chain of the antibody heavy chain constant region comprises one or more mutations, relative to SEQ ID NO:21, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:21, at one or more positions selected from Q347, Y349, L351, S354, E356, E357, S364, T366, L368, K370, N390, K392, T394, D399, D401, F405, Y407, K409, T411, and K439, numbered according to the EU numbering system. In certain embodiments, one polypeptide chain of the antibody heavy chain constant region comprises K360E and K409W substitutions relative to SEQ ID NO:21; and the other polypeptide chain of the antibody heavy chain constant region comprises Q347R, D399V and F405T substitutions relative to SEQ ID NO:21, numbered according to the EU numbering system. In certain embodiments, one polypeptide chain of the antibody heavy chain constant region comprises a Y349C substitution relative to SEQ ID NO:21; and the other polypeptide chain of the antibody heavy chain constant region comprises an S354C substitution relative to SEQ ID NO:21, numbered according to the EU numbering system.

In another aspect, the present application provides an antibody-drug conjugate comprising a protein disclosed herein and a drug moiety. In certain embodiments, the drug moiety is selected from the group consisting of auristatin, N-acetyl-γ calicheamicin, maytansinoid, pyrrolobenzodiazepine, and SN-38.

In another aspect, the present application provides an immunocytokine comprising an antigen-binding site disclosed herein and a cytokine. In certain embodiments, the cytokine is selected from the group consisting of IL-2, IL-4, IL-10, IL-12, IL-15, TNF, and IFNα.

In another aspect, the present application provides a bispecific T-cell engager comprising an antigen-binding site disclosed herein and an antigen-binding site that binds CD3.

In another aspect, the present application provides a chimeric antigen receptor (CAR) comprising:

(a) an antigen-binding site disclosed herein;

(b) a transmembrane domain; and

(c) an intracellular signaling domain.

In some embodiments, the transmembrane domain is selected from the transmembrane regions of the alpha, beta or zeta chain of the T-cell receptor, CD28, CD3 epsilon, CD45, CD4, CD5, CD8, CD9, CD16, CD22, CLEC12A, CD37, CD64, CD80, CD86, CD134, CD137, CD152, and CD154. In certain embodiments, the intracellular signaling domain comprises a primary signaling domain comprising a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In certain embodiments, the intracellular signaling domain further comprises a costimulatory signaling domain comprising a functional signaling domain of a costimulatory receptor. In certain embodiments, the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

In another aspect, the present application provides an isolated nucleic acid encoding a CAR disclosed herein.

In another aspect, the present application provides an expression vector comprising an isolated nucleic acid disclosed herein.

In another aspect, the present application provides an immune effector cell comprising a nucleic acid or an expression vector disclosed herein.

In another aspect, the present application provides an immune effector cell expressing a CAR disclosed herein. In some embodiments, the immune effector cell is a T cell. In certain embodiments, the T cell is a CD8+ T cell, a CD4+ T cell, or an NKT cell. In some embodiments, the immune effector cell is an NK cell.

In another aspect, the present application provides a pharmaceutical composition comprising a protein, an antibody-drug conjugate, an immunocytokine, a bispecific T-cell engager, or an immune effector cell disclosed herein; and a pharmaceutically acceptable carrier.

In another aspect, the present application provides a method of treating cancer, the method comprising administering to a subject in need thereof an effective amount of a protein, an antibody-drug conjugate, an immunocytokine, a bispecific T-cell engager, an immune effector cell, or a pharmaceutical composition disclosed herein.

In some embodiments, the cancer is a hematologic malignancy. In certain embodiments, the hematologic malignancy is selected from the group consisting of acute myeloid leukemia (AML), myelodysplastic syndrome (MDS), acute lymphoblastic leukemia (ALL), myeloproliferative neoplasms (MPNs), lymphoma, non-Hodgkin lymphomas, and classical Hodgkin lymphoma.

In certain embodiments, the AML is selected from undifferentiated acute myeloblastic leukemia, acute myeloblastic leukemia with minimal maturation, acute myeloblastic leukemia with maturation, acute promyelocytic leukemia (APL), acute myelomonocytic leukemia, acute myelomonocytic leukemia with eosinophilia, acute monocytic leukemia, acute erythroid leukemia, acute megakaryoblastic leukemia (AMKL), acute basophilic leukemia, acute panmyelosis with fibrosis, and blastic plasmacytoid dendritic cell neoplasm (BPDCN). In certain embodiments, the AML is characterized by expression of CLEC12A on the AML leukemia stem cells (LSCs). In certain embodiments, the LSCs further express a membrane marker selected from CD34, CD38, CD123, TIM3, CD25, CD32, and CD96.

In some embodiments, the AML is a minimal residual disease (MRD). In certain embodiments, the MRD is characterized by the presence or absence of a mutation selected from FLT3-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)). In certain embodiments, the MDS is selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U). In certain embodiments, the MDS is a primary MDS or a secondary MDS.

In some embodiments, the ALL is selected from B-cell acute lymphoblastic leukemia (B-ALL) and T-cell acute lymphoblastic leukemia (T-ALL). In certain embodiments, the MPN is selected from polycythaemia vera, essential thrombocythemia (ET), and myelofibrosis. In certain embodiments, the non-Hodgkin lymphoma is selected from B-cell lymphoma and T-cell lymphoma. In certain embodiments, wherein the lymphoma is selected from chronic lymphocytic leukemia (CLL), lymphoblastic lymphoma (LPL), diffuse large B-cell lymphoma (DLBCL), Burkitt lymphoma (BL), primary mediastinal large B-cell lymphoma (PMBL), follicular lymphoma, mantle cell lymphoma, hairy cell leukemia, plasma cell myeloma (PCM) or multiple myeloma (MM), mature T/NK neoplasms, and histiocytic neoplasms. In certain embodiments, the cancer expresses CLEC12A.

These and other aspects and advantages of the antigen-binding sites described in the present application are illustrated by the following figures, detailed description and claims.

### DESCRIPTION OF THE DRAWINGS

The application can be more completely understood with reference to the following drawings.

FIG. 1 is a set of traces showing Bio-Layer Interferometry (BLI) profiles of antibodies collected from the murine hybridomas supernatants binding to hCLEC12A.

FIG. 2 is a set of sensorgrams showing SPR profiles of antibodies collected from the murine mAb subclones binding to hCLEC12A (FIG. 2A) and cCLEC12A (FIG. 2B).

FIG. 3 is a line graph showing binding of purified CLEC12A subclones to PL21 AML cell line.

FIG. 4 is a set of sensorgrams showing SPR profiles of antibodies 16B8.C8 and 9F11.B7 binding to glycosylated, de-glycosylated, and de-sialylated hCLEC12A.

FIG. 5 is a set of sensorgrams showing SPR profiles of multispecific binding proteins containing an antigen-binding site derived from antibody 16B8.C8.

FIG. 6 are line graphs showing binding of hCLEC12A-targeting F3′-1304, F3′-1295 and a control CLEC12A-targeting multispecific binding protein to hCLEC12A-expressing cell line RMA-hCLEC12A.

FIG. 7 is a set of sensorgrams showing SPR profiles of multispecific binding proteins F3′-1295 and F3′-1602.

FIG. 8 are line graphs showing binding of hCLEC12A-targeting multispecific binding proteins F3′-1295, F3′-1602, and a control CLEC12A-targeting multispecific binding proteins to hCLEC12A-expressing cell line Ba/F3 (FIG. 8A), wild-type Ba/F3 (FIG. 8B), cancer line HL60 (FIG. 8C), and cancer line PL21 (FIG. 8D).

FIG. 9 shows flow cytometry based polyspecificity reagent (PSR) analysis of F3′-1602 and F3′-1295.

FIG. 10 are line graphs showing binding of hF3′-1602 and AB0010 to Ba/F3 expressing hCLEC12A (FIG. 10A), cancer line HL60 (FIG. 10B), and wild-type Ba/F3 (FIG. 10C).

FIG. 11 are line graphs showing binding of multispecific binding proteins derived from 9F11.B7 and hF3′-1602 and AB0010 to Ba/F3 expressing hCLEC12A (FIG. 11A) and cancer line HL60 (FIG. 11B).

FIG. 12 is a set of sensorgrams showing SPR specificity profiles of hF3′-1602 binding to human CLEC12A.

FIG. 13 are graphs demonstrating specificity of hF3′-1602 binding to recombinant hCLEC12A-His by SPR (FIG. 13A); binding to five unrelated recombinant targets by SPR (FIG. 13B); quantification of the raw data from SPR (FIG. 13C); binding to Ba/F3-CLEC12A cells by flow cytometry (FIG. 1D); and binding to Ba/F3-parental cells by flow cytometry (FIG. 13E).

FIG. 14 shows flow cytometry based polyspecificity reagent (PSR) analysis of F3′-1602.

FIG. 15 are bar graphs showing relative binding (Z score) of F3′-1602 at 1 to human CLEC12A in comparison to the entire human proteome microarray

FIG. 16 shows models of hydrophobicity patches in hF3′-1602 CLEC12A-binding arm.

FIG. 17 are bar graphs based on models of CDR-length, surface hydrophobicity, and surface charge in hF3′-1602 CLEC12A-binding arm.

FIG. 18 is a set of flow cytometry plots demonstrating sequence liability-remediated variants of hF3′-1602 binding to hCLEC12A.

### DETAILED DESCRIPTION

The present application provides antigen-binding sites that bind human CLEC12A. These antigen-binding sites bind various epitopes in an extracellular domain of CLEC12A. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating CLEC12A-associated diseases such as cancer.

The present application provides antigen-binding proteins that bind CLEC12A on a cancer cell and pharmaceutical compositions comprising such proteins, and therapeutic methods using such proteins and pharmaceutical compositions, including for the treatment of cancer. Various aspects of the antigen-binding sites described in the present application are set forth in the sections below; however, aspects of the antigen-binding sites described in the present application described in one particular section are not to be limited to any particular section.

To facilitate an understanding of the present application, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of described in the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound described in the the present application which, upon administration to a subject, is capable of providing a compound described in this application or an active metabolite or residue thereof. As is known to those of skill in the art, “salts” of the compounds described in the present application may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds described in the present application and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds described in the present application compounded with a suitable cation such as Na⁺, NH₄⁺, and NW₄⁺ (wherein W is a C₁₋₄ alkyl group), and the like.

For therapeutic use, salts of the compounds described in the present application are contemplated as being pharmaceutically acceptable. However, salts of acids and bases that are non-pharmaceutically acceptable may also find use, for example, in the preparation or purification of a pharmaceutically acceptable compound.

As used herein, “CLEC12A” (also known as CLL-1, DCAL-2, MICL, and CD371) refers to the protein of Uniprot Accession No. Q5QGZ9 and related isoforms.

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

Various features and aspects of the antigen-binding sites described in the application are discussed in more detail below.

### I. Antigen-Binding Site

In one aspect, the present application provides an antigen-binding site that binds human CLEC12A. The VH, VL, CDR, and scFv sequences of exemplary antigen-binding sites are listed in Table 1. The CDR sequences are identified according to the Chothia numbering scheme.

TABLE 1
Sequences of Exemplary Antigen-Binding Sites that Bind CLEC12A

| Clone | VH | VL |
|---|---|---|
| 16B8.C8 | EVQLQESGPGLVQPSQSLSITCT | DIQMNQSPSSLSASLGDTIAITCHA |
| | VSGFSLTNYGLHWVRQSPGKG | SQNINFWLSWYQQKPGNIPKLLIY |
| | LEWLGVIWSGGKTDYNTPFKS | EASNLHTGVPSRFSGSGSGTRFTLT |
| | RLSISKDISKNQVFFKMNSLQPN | ISSLQPEDIATYYCQQSHSYPLTFG |
| | DTAIYFCAKYDYDDSLDYWGQ | QGTKLEIK |
| | GTSVTVSS | [SEQ ID NO: 2] |
| | [SEQ ID NO: 1] | CDR1: HASQNINFWLS [SEQ ID |
| | CDR1: GFSLTNY [SEQ ID | NO: 6] |
| | NO: 11] | CDR2: EASNLHT [SEQ ID NO:7] |
| | CDR2: WSGGK [SEQ ID NO: 4] | CDR3: QQSHSYPLT [SEQ ID NO: 8] |
| | CDR3: YDYDDSLDY [SEQ ID | |
| | NO: 5] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKIPKLLIY |
| scFv-1292/ | EWIGVIWSGGKTDYNPSLKSRV | EASNLHTGVPSRFSGSGSGTRFTLT |
| scFv-1301 | TISKDTSKNQFSLKLSSVQAND | ISSLQPEDIATYYCQQSHSYPLTFG |
| (back | TAVYYCAKYDYDDSLDYWGQ | QGTKLEIK |
| mutations in | GTLVTVSS | [SEQ ID NO: 10] |
| VH and VL | [SEQ ID NO: 9] | CDR1: HASQNINFWLS [SEQ ID |
| underlined) | CDR1: GFSLTNY [SEQ ID NO: 11] | NO: 6] |
| | CDR2: WSGGK [SEQ ID NO: 4] | CDR2: EASNLHT [SEQ ID NO: 7] |
| | CDR3: YDYDDSLDY [SEQ ID | CDR3: QQSHSYPLT [SEQ ID NO: 8] |
| | NO: 5] | |
| scFv of | scFv-1292 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISKDTSKNQFSLKLSSVQANDTAVYYCAK | |
| scFv-1292/ | YDYDDSLDYWGQGTLVTVSSGGGGSGGGGSGGGGSGGGGSDIQMT | |
| scFv-1301 | QSPSSLSASVGDRVTITCHASQNINFWLSWYQQKPGKIPKLLIYEASNL | |
| | HTGVPSRFSGSGSGTRFTLTISSLQPEDIATYYCQQSHSYPLTFGCGTKL | |
| | EIK [SEQ ID NO: 3] | |
| | scFv-1301 (VL-VH): | |
| | DIQMTQSPSSLSASVGDRVTITCHASQNINFWLSWYQQKPGKIPKLLIY | |
| | EASNLHTGVPSRFSGSGSGTRFTLTISSLQPEDIATYYCQQSHSYPLTFG | |
| | CGTKLEIKGGGGSGGGGSGGGGSGGGGSQLQLQESGPGLVKPSETLS | |
| | LTCTVSGFSLTNYGLHWIRQPPGKCLEWIGVIWSGGKTDYNPSLKSRV | |
| | TISKDTSKNQFSLKLSSVQANDTAVYYCAKYDYDDSLDYWGQGTLVT | |
| | VSS | |
| | [SEQ ID NO: 12] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKIPKLLIY |
| scFv-1293/ | EWIGVIWSGGKTDYNPSLKSRV | EASNLHTGVPSRFSGSGSGTRFTLT |
| scFv-1302 | TISVDTSKNQFSLKLSSVQAND | ISSLQPEDIATYYCQQSHSYPLTFG |
| (back | TAVYYCAKYDYDDSLDYWGQ | QGTKLEIK |
| mutations in | GTLVTVSS | [SEQ ID NO: 10] |
| VHand VL | [SEQ ID NO: 13] | CDR1: HASQNINFWLS [SEQ ID |
| underlined) | CDR1: GFSLTNY [SEQ ID | NO: 6] |
| | NO: 11] | CDR2: EASNLHT [SEQ ID NO: 7] |
| | CDR2: WSGGK [SEQ ID NO: 4] | CDR3: QQSHSYPLT [SEQ ID NO: 8] |
| | CDR3: YDYDDSLDY [SEQ ID | |
| | NO: 5] | |
| scFv of | scFv-1293 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISVDTSKNQFSLKLSSVQANDTAVYYCAK | |
| scFv-1293/ | YDYDDSLDYWGQGTLVTVSSGGGGSGGGGSGGGGSGGGGSDIQMT | |
| scFv-1302 | QSPSSLSASVGDRVTITCHASQNINFWLSWYQQKPGKIPKLLIYEASNL | |
| | HTGVPSRFSGSGSGTRFTLTISSLQPEDIATYYCQQSHSYPLTFGCGTKL | |
| | EIK | |
| | [SEQ ID NO: 15] | |
| | scFv-1302 (VL-VH): | |
| | DIQMTQSPSSLSASVGDRVTITCHASQNINFWLSWYQQKPGKIPKLLIY | |
| | EASNLHTGVPSRFSGSGSGTRFTLTISSLQPEDIATYYCQQSHSYPLTFG | |
| | CGTKLEIKGGGGSGGGGSGGGGSGGGGSQLQLQESGPGLVKPSETLS | |
| | LTCTVSGFSLTNYGLHWIRQPPGKCLEWIGVIWSGGKTDYNPSLKSRV | |
| | TISVDTSKNQFSLKLSSVQANDTAVYYCAKYDYDDSLDYWGQGTLVT | |
| | VSS | |
| | [SEQ ID NO: 16] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKIPKLLIY |
| scFv-1294/ | EWIGVIWSGGKTDYNPSLKSRV | EASNLHTGVPSRFSGSGSGTRFTLT |
| scFv-1303 | TISKDTSKNQFSLKLSSVTANDT | ISSLQPEDIATYYCQQSHSYPLTFG |
| (back | AVYYCAKYDYDDSLDYWGQG | QGTKLEIK |
| mutations in | TLVTVSS | |
| VH and VL | [SEQ ID NO: 110] | [SEQ ID NO: 10] |
| underlined) | CDR1: GFSLTNY [SEQ ID | CDR1: HASQNINFWLS [SEQ ID |
| | NO: 11] | NO: 6] |
| | CDR2: WSGGK [SEQ ID NO: 4] | CDR2: EASNLHT [SEQ ID NO: 7] |
| | CDR3: YDYDDSLDY [SEQ ID | CDR3: QQSHSYPLT [SEQ ID NO: 8] |
| | NO: 5] | |
| scFv of | scFv-1294 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISKDTSKNQFSLKLSSVTANDTAVYYCAK | |
| scFv-1294/ | YDYDDSLDYWGQGTLVTVSSGGGGSGGGGSGGGGSGGGGSDIQMT | |
| scFv-1303 | QSPSSLSASVGDRVTITCHASQNINFWLSWYQQKPGKIPKLLIYEASNL | |
| | HTGVPSRFSGSGSGTRFTLTISSLQPEDIATYYCQQSHSYPLTFGCGTKL | |
| | EIK | |
| | [SEQ ID NO: 19] | |
| | scFv-1303 (VL-VH): | |
| | DIQMTQSPSSLSASVGDRVTITCHASQNINFWLSWYQQKPGKIPKLLIY | |
| | EASNLHTGVPSRFSGSGSGTRFTLTISSLQPEDIATYYCQQSHSYPLTFG | |
| | CGTKLEIKGGGGSGGGGSGGGGSGGGGSQLQLQESGPGLVKPSETLS | |
| | LTCTVSGFSLTNYGLHWIRQPPGKCLEWIGVIWSGGKTDYNPSLKSRV | |
| | TISKDTSKNQFSLKLSSVTANDTAVYYCAKYDYDDSLDYWGQGTLVT | |
| | VSS | |
| | [SEQ ID NO: 20] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKIPKLLIY |
| scFv-1295/ | EWIGVIWSGGKTDYNPSLKSRV | EASNLHTGVPSRFSGSGSGTRFTLT |
| scFv-1304 | TISKDTSKNQFSLKLSSVQAAD | ISSLQPEDIATYYCQQSHSYPLTFG |
| (back | TAVYYCAKYDYDDSLDYWGQ | QGTKLEIK |
| mutations in | GTLVTVSS | [SEQ ID NO: 10] |
| VHand VL | [SEQ ID NO: 45] | CDR1: HASQNINFWLS [SEQ ID |
| underlined) | CDR1: GFSLTNY [SEQ ID | NO: 6] |
| | NO: 11] | CDR2: EASNLHT [SEQ ID NO: 7] |
| | CDR2: WSGGK [SEQ ID NO: 4] | CDR3: QQSHSYPLT [SEQ ID NO: 8] |
| | CDR3: YDYDDSLDY [SEQ ID | |
| | NO: 5] | |
| scFv of | scFv-1295 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISKDTSKNQFSLKLSSVQAADTAVYYCAK | |
| scFv-1295/ | YDYDDSLDYWGQGTLVTVSSGGGGSGGGGSGGGGSGGGGSDIQMT | |
| scFv-1304 | QSPSSLSASVGDRVTITCHASQNINFWLSWYQQKPGKIPKLLIYEASNL | |
| | HTGVPSRFSGSGSGTRFTLTISSLQPEDIATYYCQQSHSYPLTFGCGTKL | |
| | EIK | |
| | [SEQ ID NO: 23] | |
| | scFv-1304 (VL-VH): | |
| | DIQMTQSPSSLSASVGDRVTITCHASQNINFWLSWYQQKPGKIPKLLIY | |
| | EASNLHTGVPSRFSGSGSGTRFTLTISSLQPEDIATYYCQQSHSYPLTFG | |
| | CGTKLEIKGGGGSGGGGSGGGGSGGGGSQLQLQESGPGLVKPSETLS | |
| | LTCTVSGFSLTNYGLHWIRQPPGKCLEWIGVIWSGGKTDYNPSLKSRV | |
| | TISKDTSKNQFSLKLSSVQAADTAVYYCAKYDYDDSLDYWGQGTLVT | |
| | VSS | |
| | [SEQ ID NO: 24] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKIPKLLIY |
| scFv-1296/ | EWIGVIWSGGKTDYNPSLKSRV | EASNLHTGVPSRFSGSGSGTRFTLT |
| scFv-1305 | TISKDTSKNQFSLKLSSVQAND | ISSLQPEDIATYYCQQSHSYPLTFG |
| (back | TAVYYCARYDYDDSLDYWGQG | QGTKLEIK |
| mutations in | TLVTVSS | [SEQ ID NO: 10] |
| VH and VL | [SEQ ID NO: 122] | CDR1: HASQNINFWLS [SEQ ID |
| underlined) | CDR1: GFSLTNY [SEQ ID | NO: 6] |
| | NO: 11] | CDR2: EASNLHT [SEQ ID NO: 7] |
| | CDR2: WSGGK [SEQ ID NO: 4] | CDR3: QQSHSYPLT [SEQ ID NO: 8] |
| | CDR3: YDYDDSLDY [SEQ ID | |
| | NO: 5] | |
| scFv of | scFv-1296 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISKDTSKNQFSLKLSSVQANDTAVYYCAR | |
| scFv-1296/ | YDYDDSLDYWGQGTLVTVSSGGGGSGGGGSGGGGSGGGGSDIQMT | |
| scFv-1305 | QSPSSLSASVGDRVTITCHASQNINFWLSWYQQKPGKIPKLLIYEASNL | |
| | HTGVPSRFSGSGSGTRFTLTISSLQPEDIATYYCQQSHSYPLTFGCGTKL | |
| | EIK | |
| | [SEQ ID NO: 27] | |
| | scFv-1305 (VL-VH): | |
| | DIQMTQSPSSLSASVGDRVTITCHASQNINFWLSWYQQKPGKIPKLLIY | |
| | EASNLHTGVPSRFSGSGSGTRFTLTISSLQPEDIATYYCQQSHSYPLTFG | |
| | CGTKLEIKGGGGSGGGGSGGGGSGGGGSQLQLQESGPGLVKPSETLS | |
| | LTCTVSGFSLTNYGLHWIRQPPGKCLEWIGVIWSGGKTDYNPSLKSRV | |
| | TISKDTSKNQFSLKLSSVQANDTAVYYCARYDYDDSLDYWGQGTLVT | |
| | VSS | |
| | [SEQ ID NO: 28] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKAPKLLIY |
| scFv-1297/ | EWIGVIWSGGKTDYNPSLKSRV | EASNLHTGVPSRFSGSGSGTRFTLT |
| scFv-1306 | TISKDTSKNQFSLKLSSVQAND | ISSLQPEDIATYYCQQSHSYPLTFG |
| (back | TAVYYCAKYDYDDSLDYWGQ | QGTKLEIK |
| mutations in | GTLVTVSS | [SEQ ID NO: 30] |
| VH and VL | [SEQ ID NO: 9] | CDR1: HASQNINFWLS [SEQ ID |
| underlined) | CDR1: GFSLTNY [SEQ ID | NO: 6] |
| | NO: 11] | CDR2: EASNLHT [SEQ ID NO: 7] |
| | CDR2: WSGGK [SEQ ID NO: 4] | CDR3: QQSHSYPLT [SEQ ID NO: 8] |
| | CDR3: YDYDDSLDY [SEQ ID | |
| | NO: 5] | |
| scFv of | scFv-1297 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISKDTSKNQFSLKLSSVQANDTAVYYCAK | |
| scFv-1297/ | YDYDDSLDYWGQGTLVTVSSGGGGSGGGGSGGGGSGGGGSDIQMT | |
| scFv-1306 | QSPSSLSASVGDRVTITCHASQNINFWLSWYQQKPGKAPKLLIYEASN | |
| | LHTGVPSRFSGSGSGTRFTLTISSLQPEDIATYYCQQSHSYPLTFGCGTK | |
| | LEIK | |
| | [SEQ ID NO: 31] | |
| | scFv-1306 (VL-VH): | |
| | DIQMTQSPSSLSASVGDRVTITCHASQNINFWLSWYQQKPGKAPKLLI | |
| | YEASNLHTGVPSRFSGSGSGTRFTLTISSLQPEDIATYYCQQSHSYPLTF | |
| | GCGTKLEIKGGGGSGGGGSGGGGSGGGGSQLQLQESGPGLVKPSETL | |
| | SLTCTVSGFSLTNYGLHWIRQPPGKCLEWIGVIWSGGKTDYNPSLKSR | |
| | VTISKDTSKNQFSLKLSSVQANDTAVYYCAKYDYDDSLDYWGQGTLV | |
| | TVSS | |
| | [SEQ ID NO: 32] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKIPKLLIY |
| scFv-1298/ | EWIGVIWSGGKTDYNPSLKSRV | EASNLHTGVPSRFSGSGSGTDFTLT |
| scFv-1307 | TISKDTSKNQFSLKLSSVQAND | ISSLQPEDIATYYCQQSHSYPLTFG |
| (back | TAVYYCAKYDYDDSLDYWGQ | QGTKLEIK |
| mutations in | GTLVTVSS | [SEQ ID NO: 34] |
| VH and VL | [SEQ ID NO: 9] | CDR1: HASQNINFWLS [SEQ ID |
| underlined) | CDR1: GFSLTNY [SEQ ID | NO: 6] |
| | NO: 11] | CDR2: EASNLHT [SEQ ID NO: 7] |
| | CDR2: WSGGK [SEQ ID NO: 4] | CDR3: QQSHSYPLT [SEQ ID NO: 8] |
| | CDR3: YDYDDSLDY [SEQ ID | |
| | NO: 5] | |
| scFv of | scFv-1298 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISKDTSKNQFSLKLSSVQANDTAVYYCAK | |
| scFv-1298/ | YDYDDSLDYWGQGTLVTVSSGGGGSGGGGSGGGGSGGGGSDIQMT | |
| scFv-1307 | QSPSSLSASVGDRVTITCHASQNINFWLSWYQQKPGKIPKLLIYEASNL | |
| | HTGVPSRFSGSGSGTDFTLTISSLQPEDIATYYCQQSHSYPLTFGCGTKL | |
| | EIK | |
| | [SEQ ID NO: 35] | |
| | scFv-1307 (VL-VH): | |
| | DIQMTQSPSSLSASVGDRVTITCHASQNINFWLSWYQQKPGKIPKLLIY | |
| | EASNLHTGVPSRFSGSGSGTDFTLTISSLQPEDIATYYCQQSHSYPLTFG | |
| | CGTKLEIKGGGGSGGGGSGGGGSGGGGSQLQLQESGPGLVKPSETLS | |
| | LTCTVSGFSLTNYGLHWIRQPPGKCLEWIGVIWSGGKTDYNPSLKSRV | |
| | TISKDTSKNQFSLKLSSVQANDTAVYYCAKYDYDDSLDYWGQGTLVT | |
| | VSS | |
| | [SEQ ID NO: 36] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKIPKLLIY |
| scFv-1299/ | EWIGVIWSGGKTDYNPSLKSRV | EASNLHTGVPSRFSGSGSGTRFTLT |
| scFv-1308 | TISKDTSKNQFSLKLSSVQAND | ISSLQPEDFATYYCQQSHSYPLTFG |
| | TAVYYCAKYDYDDSLDYWGQ | QGTKLEIK |
| | GTLVTVSS | [SEQ ID NO: 38] |
| | [SEQ ID NO: 9] | CDR1: HASQNINFWLS |
| | CDR1: GFSLTNY [SEQ ID | [SEQ ID NO: 6] |
| | NO: 11] | CDR2: EASNLHT [SEQ ID NO: 7] |
| | CDR2: WSGGK [SEQ ID NO: 4] | CDR3: QQSHSYPLT [SEQ ID NO: 8] |
| | CDR3: YDYDDSLDY [SEQ ID | |
| | NO: 5] | |
| scFv of | scFv-1299 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISKDTSKNQFSLKLSSVQANDTAVYYCAK | |
| scFv-1299/ | YDYDDSLDYWGQGTLVTVSSGGGGSGGGGSGGGGSGGGGSDIQMT | |
| scFv-1308 | QSPSSLSASVGDRVTITCHASQNINFWLSWYQQKPGKIPKLLIYEASNL | |
| | HTGVPSRFSGSGSGTRFTLTISSLQPEDFATYYCQQSHSYPLTFGCGTKL | |
| | EIK | |
| | [SEQ ID NO: 39] | |
| | scFv-1308 (VL-VH): | |
| | DIQMTQSPSSLSASVGDRVTITCHASQNINFWLSWYQQKPGKIPKLLIY | |
| | EASNLHTGVPSRFSGSGSGTRFTLTISSLQPEDFATYYCQQSHSYPLTFG | |
| | CGTKLEIKGGGGSGGGGSGGGGSGGGGSQLQLQESGPGLVKPSETLS | |
| | LTCTVSGFSLTNYGLHWIRQPPGKCLEWIGVIWSGGKTDYNPSLKSRV | |
| | TISKDTSKNQFSLKLSSVQANDTAVYYCAKYDYDDSLDYWGQGTLVT | |
| | VSS | |
| | [SEQ ID NO: 40] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKAPKLLIY |
| scFv-1300/ | EWIGVIWSGGKTDYNPSLKSRV | EASNLHTGVPSRFSGSGSGTDFTLT |
| scFv-1309 | TISVDTSKNQFSLKLSSVTAADT | ISSLQPEDFATYYCQQSHSYPLTFG |
| | AVYYCARYDYDDSLDYWGQG | QGTKLEIK |
| | TLVTVSS | [SEQ ID NO: 42] |
| | [SEQ ID NO: 41] | CDR1: HASQNINFWLS [SEQ ID |
| | CDR1: GFSLTNY [SEQ ID | NO: 6] |
| | NO: 11] | CDR2: EASNLHT [SEQ ID NO: 7] |
| | CDR2: WSGGK [SEQ ID NO: 4] | CDR3: QQSHSYPLT [SEQ ID NO: 8] |
| | CDR3: YDYDDSLDY [SEQ ID | |
| | NO: 5] | |
| scFv of | scFv-1300 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISVDTSKNQFSLKLSSVTAADTAVYYCAR | |
| scFv-1300/ | YDYDDSLDYWGQGTLVTVSSGGGGSGGGGSGGGGSGGGGSDIQMT | |
| scFv-1309 | QSPSSLSASVGDRVTITCHASQNINFWLSWYQQKPGKAPKLLIYEASN | |
| | LHTGVPSRFSGSGSGTDFTLTISSLQPEDFATYYCQQSHSYPLTFGCGTK | |
| | LEIK | |
| | [SEQ ID NO: 43] | |
| | scFv-1309 (VL-VH): | |
| | DIQMTQSPSSLSASVGDRVTITCHASQNINFWLSWYQQKPGKAPKLLI | |
| | YEASNLHTGVPSRFSGSGSGTDFTLTISSLQPEDFATYYCQQSHSYPLTF | |
| | GCGTKLEIKGGGGSGGGGSGGGGSGGGGSQLQLQESGPGLVKPSETL | |
| | SLTCTVSGFSLTNYGLHWIRQPPGKCLEWIGVIWSGGKTDYNPSLKSR | |
| | VTISVDTSKNQFSLKLSSVTAADTAVYYCARYDYDDSLDYWGQGTLV | |
| | TVSS | |
| | [SEQ ID NO: 44] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKAPKLLIY |
| scFv-1602/ | EWIGVIWSGGKTDYNPSLKSRV | EASNLHTGVPSRFSGSGSGTRFTLT |
| scFv-2061 | TISKDTSKNQFSLKLSSVQAAD | ISSLQPEDIATYYCQQSHSYPLTFG |
| | TAVYYCAKYDYDDSLDYWGQ | GGTKLEIK |
| | GTLVTVSS [SEQ ID NO: 45] | [SEQ ID NO: 140] |
| | CDR1: GFSLTNY [SEQ ID | CDR1: HASQNINFWLS [SEQ ID |
| | NO: 11] | NO: 6] |
| | CDR2: WSGGK [SEQ ID NO: 4] | CDR2: EASNLHT [SEQ ID NO: 7] |
| | CDR3: YDYDDSLDY [SEQ ID | CDR3: QQSHSYPLT [SEQ ID NO: 8] |
| | NO: 5] | |
| scFv of | scFv-1602 (VH-VL): | |
| humanized | QLQLQESGPGLVKPSETLSLTCTVSGFSLTNYGLHWIRQPPGKCLEWIG | |
| 16B8.C8 | VIWSGGKTDYNPSLKSRVTISKDTSKNQFSLKLSSVQAADTAVYYCAK | |
| scFv-1602/ | YDYDDSLDYWGQGTLVTVSSGGGGSGGGGSGGGGSGGGGSDIQMT | |
| scFv-2061 | QSPSSLSASVGDRVTITCHASQNINFWLSWYQQKPGKAPKLLIYEASN | |
| | LHTGVPSRFSGSGSGTRFTLTISSLQPEDIATYYCQQSHSYPLTFGCGTK | |
| | LEIK | |
| | [SEQ ID NO: 47] | |
| | scFv-2061 (VL-VH): | |
| | DIQMTQSPSSLSASVGDRVTITCHASQNINFWLSWYQQKPGKAPKLLI | |
| | YEASNLHTGVPSRFSGSGSGTRFTLTISSLQPEDIATYYCQQSHSYPLTF | |
| | GCGTKLEIKGGGGSGGGGSGGGGSGGGGSQLQLQESGPGLVKPSETL | |
| | SLTCTVSGFSLTNYGLHWIRQPPGKCLEWIGVIWSGGKTDYNPSLKSR | |
| | VTISKDTSKNQFSLKLSSVQAADTAVYYCAKYDYDDSLDYWGQGTLV | |
| | TVSS | |
| | [SEQ ID NO: 48] | |
| Humanized | QLQLQESGPGLVKPSETLSLTCT | DIQMTQSPSSLSASVGDRVTITCHA |
| 16B8.C8 | VSGFSLTNYGLHWIRQPPGKGL | SQNINFWLSWYQQKPGKX₁PKLLI |
| consensus | EWIGVIWSGGKTDYNPSLKSRV | YEASNLHTGVPSRFSGSGSGTX₂FT |
| | TISX₁DTSKNQFSLKLSSVX₂AX₃ | LTISSLQPEDX₃ATYYCQQSHSYPL |
| | DTAVYYCAX₄YDYDDSLDYWG | TFGX₄GTKLEIK |
| | QGTLVTVSS | where X₁ is A or I, X₂ |
| | where X₁ is V or K, X₂ | is D or R, X₃ is F or I, |
| | is T or Q, X₃ is A or N, | and X₄ is Q or G |
| | and X₄ is R or K | [SEQ ID NO: 17] |
| | [SEQ ID NO: 49] | CDR1: HASQNINFWLS |
| | CDR1: GFSLTNY | [SEQ ID NO: 6] |
| | [SEQ ID NO: 11] | CDR2: EASNLHT [SEQ ID NO: 7] |
| | CDR2: WSGGK [SEQ ID NO: 4] | CDR3: QQSHSYPLT [SEQ ID NO: 8] |
| | CDR3: YDYDDSLDY | |
| | [SEQ ID NO: 5] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKAPKLLIY |
| AB0305/ | EWIGVIWVGGATDYNPSLKSR | EASNLHTGVPSRFSGSGSGTDFTLT |
| AB5030 | VTISVDTSKNQFSLKLSSVQAA | ISSLQPEDFATYYCQQSHSYPLTFG |
| (Cysteine | DTAVYYCAKGDYGDTLDYWG | SGTKLEIK |
| heterodi- | QGTLVTVSS | [SEQ ID NO: 129] |
| merization | [SEQ ID NO: 128] | DIQMTQSPSSLSASVGDRVTITCHA |
| mutations | QLQLQESGPGLVKPSETLSLTCT | SQNINFWLSWYQQKPGKAPKLLIY |
| are | VSGFSLTNYGLHWIRQPPGKCL | EASNLHTGVPSRFSGSGSGTDFTLT |
| underlined. | EWIGVIWVGGATDYNPSLKSRV | ISSLQPEDFATYYCQQSHSYPLTFG |
| Such muta- | TISVDTSKNQFSLKLSSVQAAD | CGTKLEIK |
| tions can | TAVYYCAKGDYGDTLDYWGQ | [SEQ ID NO: 148] |
| facilitate | GTLVTVSS | CDR1: HASQNINFWLS [SEQ ID |
| formation | [SEQ ID NO: 147] | NO: 6] |
| of a disul- | CDR1: GFSLTNY [SEQ ID | CDR2: EASNLHT [SEQ ID NO: 7] |
| fide bridge | NO: 11] | CDR3: QQSHSYPLT [SEQ ID NO: 8] |
| between the | CDR2: WVGGA [SEQ ID NO: 130] | |
| VH and VL | CDR3: GDYGDTLDY [SEQ ID | |
| of the | NO: 131] | |
| scFv.) | | |
| scFv of | AB0305 (VH-VL): | |
| humanized | QLQLQESGPGLVKPSETLSLTCTVSGFSLTNYGLHWIRQPPGKCLEWI | |
| 16B8.C8 | GVIWVGGATDYNPSLKSRVTISVDTSKNQFSLKLSSVQAADTAVYYC | |
| AB0305/ | AKGDYGDTLDYWGQGTLVTVSSGGGGSGGGGSGGGGSGGGGSDIQ | |
| AB5030 | MTQSPSSLSASVGDRVTITCHASQNINFWLSWYQQKPGKAPKLLIYEA | |
| | SNLHTGVPSRFSGSGSGTDFTLTISSLQPEDFATYYCQQSHSYPLTFGCG | |
| | TKLEIK | |
| | [SEQ ID NO: 132] | |
| | AB5030 (VL-VH): | |
| | DIQMTQSPSSLSASVGDRVTITCHASQNINFWLSWYQQKPGKAPKLLI | |
| | YEASNLHTGVPSRFSGSGSGTDFTLTISSLQPEDFATYYCQQSHSYPLTF | |
| | GCGTKLEIKGGGGSGGGGSGGGGSGGGGSQLQLQESGPGLVKPSETL | |
| | SLTCTVSGFSLTNYGLHWIRQPPGKCLEWIGVIWVGGATDYNPSLKSR | |
| | VTISVDTSKNQFSLKLSSVQAADTAVYYCAKGDYGDTLDYWGQGTL | |
| | VTVSS | |
| | [SEQ ID NO: 133] | |
| Humanized | QLQLQESGPGLVKPSETLSLTCT | DIQMTQSPSSLSASVGDRVTITCHA |
| 16B8.C8 in | VSGFSLTNYGLHWIRQPPGKGL | SQNINFWLSWYQQKPGKAPKLLIY |
| AB0147/ | EWIGVILSGGWTDYNPSLKSRV | EASNLHTGVPSRFSGSGSGTRFTLT |
| AB7410 | TISKDTSKNQFSLKLSSVQAAD | ISSLQPEDIATYYCQQSHSYPLTFG |
| | TAVYYCAKGDYGDALDYWGQ | SGTKLEIK |
| | GTLVTVSS | [SEQ ID NO: 135] |
| | [SEQ ID NO: 134] | CDR1: HASQNINFWLS [SEQ ID |
| | CDR1: GFSLTNY [SEQ ID | NO: 6] |
| | NO: 11] | CDR2: EASNLHT [SEQ ID NO: 7] |
| | CDR2: LSGGW [SEQ ID NO: 136] | CDR3: QQSHSYPLT [SEQ ID NO: 8] |
| | CDR3: GDYGDALDY [SEQ ID | |
| | NO: 137] | |
| scFv of | AB0147 (VH-VL) | |
| humanized | QLQLQESGPGLVKPSETLSLTCTVSGFSLTNYGLHWIRQPPGKCLEWI | |
| 16B8.C8 | GVILSGGWTDYNPSLKSRVTISKDTSKNQFSLKLSSVQAADTAVYYC | |
| | AKGDYGDALDYWGQGTLVTVSSGGGGSGGGGSGGGGSGGGGSDIQ | |
| AB0147/ | MTQSPSSLSASVGDRVTITCHASQNINFWLSWYQQKPGKAPKLLIYEA | |
| AB7410 | SNLHTGVPSRFSGSGSGTRFTLTISSLQPEDIATYYCQQSHSYPLTFGCG | |
| | TKLEIK | |
| | [SEQ ID NO: 138] | |
| | AB7410 (VL-VH) | |
| | DIQMTQSPSSLSASVGDRVTITCHASQNINFWLSWYQQKPGKAPKLLI | |
| | YEASNLHTGVPSRFSGSGSGTRFTLTISSLQPEDIATYYCQQSHSYPLTF | |
| | GCGTKLEIKGGGGSGGGGSGGGGSGGGGSQLQLQESGPGLVKPSETL | |
| | SLTCTVSGFSLTNYGLHWIRQPPGKCLEWIGVILSGGWTDYNPSLKSR | |
| | VTISKDTSKNQFSLKLSSVQAADTAVYYCAKGDYGDALDYWGQGTL | |
| | VTVSS | |
| | [SEQ ID NO: 139] | |
| 9F11.B7 | EVQLQESGGGLVQPGGSRKLSC | DIKMTQSPSSMYASLGERVTITCK |
| | AASGFTFNSFGMHWVRQAPEK | ASQDIYNYLSWFQLKPGKSPRPLI |
| | GLEWVAFISSGSTSIYYANTVK | YRANILVSGVPSKFSGSGSGQDYS |
| | GRFTISRDNPKNTLFLQMTSLRS | LTINSLEYEDLGIYYCLQFDAFPFT |
| | EDTAMYYCARDGYPTGGAMD | FGSGTKLEIK |
| | YWGQGTSVTVSS [SEQ ID | [SEQ ID NO: 61] |
| | NO: 60] | CDR1: KASQDIYNYLS [SEQ ID |
| | CDR1: GFTFNSF [SEQ ID NO: 59] | NO: 65] |
| | CDR2: SSGSTS [SEQ ID NO: 63] | CDR2: RANILVS [SEQ ID NO: 66] |
| | CDR3: DGYPTGGAMDY [SEQ | CDR3: LQFDAFPFT [SEQ ID NO: 67] |
| | ID NO: 54] | |
| Humanized | EVQLVESGGGVVQPGGSLRLSC | DIQMTQSPSSLSASVGDRVTITCKA |
| 9F11.B7 in | AASGFTFNSFGMHWVRQAPGK | SQDIYNYLSWFQQKPGKAPKPLIY |
| AB0191/ | GLEWVAFISSGSTSIYYANTVKG | RANILVSGVPSRFSGSGSGQDYTFT |
| AB0185 | RFTISRDNSKNTLYLQMNSLRA | ISSLQPEDIATYYCLQFDAFPFTFGS |
| (back | EDTAVYYCARDGYPTGGAMDY | GTKLEIK |
| mutations in | WGQGTSVTVSS | [SEQ ID NO: 69] |
| VH and VL | [SEQ ID NO: 29] | CDR1: KASQDIYNYLS [SEQ ID |
| underlined) | CDR1: GFTFNSF [SEQ ID NO: 59] | NO: 65] |
| | CDR2: SSGSTS [SEQ ID NO: 63] | CDR2: RANILVS [SEQ ID NO: 66] |
| | CDR3: DGYPTGGAMDY [SEQ | CDR3: LQFDAFPFT [SEQ ID NO: 67] |
| | ID NO: 54] | |
| scFv of | AB0191 (VH-VL): | |
| humanized | EVQLVESGGGVVQPGGSLRLSCAASGFTFNSFGMHWVRQAPGKCLE | |
| 9F11.B7 | WVAFISSGSTSIYYANTVKGRFTISRDNSKNTLYLQMNSLRAEDTAVY | |
| AB0191/ | YCARDGYPTGGAMDYWGQGTSVTVSSGGGGSGGGGSGGGGSGGGG | |
| AB0185 | SDIQMTQSPSSLSASVGDRVTITCKASQDIYNYLSWFQQKPGKAPKPLI | |
| | YRANILVSGVPSRFSGSGSGQDYTFTISSLQPEDIATYYCLQFDAFPFTF | |
| | GCGTKLEIK | |
| | [SEQ ID NO: 51] | |
| | AB0185 (VL-VH): | |
| | DIQMTQSPSSLSASVGDRVTITCKASQDIYNYLSWFQQKPGKAPKPLIY | |
| | RANILVSGVPSRFSGSGSGQDYTFTISSLQPEDIATYYCLQFDAFPFTFG | |
| | CGTKLEIKGGGGSGGGGSGGGGSGGGGSEVQLVESGGGVVQPGGSL | |
| | RLSCAASGFTFNSFGMHWVRQAPGKCLEWVAFISSGSTSIYYANTVKG | |
| | RFTISRDNSKNTLYLQMNSLRAEDTAVYYCARDGYPTGGAMDYWGQ | |
| | GTSVTVSS | |
| | [SEQ ID NO: 52] | |
| Humanized | EVQLVESGGGVVQPGGSLRLSC | DIQMTQSPSSLSASVGDRVTITCKA |
| 9F11.B7 in | AASGFTFNAFGMHWVRQAPGK | SQDIYNYLSWFQQKPGKAPKPLIY |
| AB0192/ | GLEWVAFISSGSTSIYYANTVKG | RANILVSGVPSRFSGSGSGQDYTFT |
| AB0186 | RFTISRDNSKNTLYLQMNSLRA | ISSLQPEDIATYYCLQFDAFPFTFGS |
| (back | EDTAVYYCARDGYPTGGAMDY | GTKLEIK |
| mutations in | WGQGTSVTVSS | [SEQ ID NO: 69] |
| VH and VL | [SEQ ID NO: 14] | CDR1: KASQDIYNYLS [SEQ ID |
| underlined, | CDR1: GFTFNAF [SEQ ID NO: 62] | NO: 65] |
| sequence | CDR2: SSGSTS [SEQ ID NO: 63] | CDR2: RANILVS [SEQ ID NO: 66] |
| liability | CDR3: DGYPTGGAMDY [SEQ | CDR3: LQFDAFPFT [SEQ ID NO: 67] |
| replacement | ID NO: 54] | |
| italicized) | | |
| scFv of | AB0192 (VH-VL): | |
| humanized | EVQLVESGGGVVQPGGSLRLSCAASGFTFNAFGMHWVRQAPGKCLE | |
| 9F11.B7 | WVAFISSGSTSIYYANTVKGRFTISRDNSKNTLYLQMNSLRAEDTAVY | |
| AB0192/ | YCARDGYPTGGAMDYWGQGTSVTVSSGGGGSGGGGSGGGGSGGGG | |
| AB0186 | SDIQMTQSPSSLSASVGDRVTITCKASQDIYNYLSWFQQKPGKAPKPLI | |
| | YRANILVSGVPSRFSGSGSGQDYTFTISSLQPEDIATYYCLQFDAFPFTF | |
| | GCGTKLEIK | |
| | [SEQ ID NO: 70] | |
| | AB0186 (VL-VH): | |
| | DIQMTQSPSSLSASVGDRVTITCKASQDIYNYLSWFQQKPGKAPKPLIY | |
| | RANILVSGVPSRFSGSGSGQDYTFTISSLQPEDIATYYCLQFDAFPFTFG | |
| | CGTKLEIKGGGGSGGGGSGGGGSGGGGSEVQLVESGGGVVQPGGSL | |
| | RLSCAASGFTFNAFGMHWVRQAPGKCLEWVAFISSGSTSIYYANTVK | |
| | GRFTISRDNSKNTLYLQMNSLRAEDTAVYYCARDGYPTGGAMDYWG | |
| | QGTSVTVSS | |
| | [SEQ ID NO: 71] | |
| Humanized | EVQLVESGGGVVQPGGSLRLSC | DIQMTQSPSSLSASVGDRVTITCKA |
| 9F11.B7 in | AASGFTFNSFGMHWVRQAPGK | SQDIYNYLSWFQQKPGKAPKPLIY |
| AB0193/ | GLEWVAFISSGSTSIYYANTVKG | RANILVSGVPSRFSGSGSGQDYTFT |
| AB0187 | RFTISRDNSKNTLYLQMNSLRA | ISSLQPEDIATYYCLQFDAFPFTFGS |
| (back | EDTAVYYCARSGYPTGGAMDY | GTKLEIK |
| mutations in | WGQGTSVTVSS | [SEQ ID NO: 69] |
| VH and VL | [SEQ ID NO: 76] | CDR1: KASQDIYNYLS [SEQ ID |
| underlined, | CDR1: GFTFNSF [SEQ ID NO: 59] | NO: 65] |
| sequence | CDR2: SSGSTS [SEQ ID NO: 63] | CDR2: RANILVS [SEQ ID NO: 66] |
| liability | CDR3: SGYPTGGAMDY [SEQ ID | CDR3: LQFDAFPFT [SEQ ID NO: 67] |
| replacement | NO: 79] | |
| italicized) | | |
| scFv of | AB0193 (VH-VL): | |
| humanized | EVQLVESGGGVVQPGGSLRLSCAASGFTFNSFGMHWVRQAPGKCLE | |
| 9F11.B7 | WVAFISSGSTSIYYANTVKGRFTISRDNSKNTLYLQMNSLRAEDTAVY | |
| AB0193/ | YCARSGYPTGGAMDYWGQGTSVTVSSGGGGSGGGGSGGGGSGGGG | |
| AB0187 | SDIQMTQSPSSLSASVGDRVTITCKASQDIYNYLSWFQQKPGKAPKPLI | |
| | YRANILVSGVPSRFSGSGSGQDYTFTISSLQPEDIATYYCLQFDAFPFTF | |
| | GCGTKLEIK | |
| | [SEQ ID NO: 74] | |
| | AB0187 (VL-VH): | |
| | DIQMTQSPSSLSASVGDRVTITCKASQDIYNYLSWFQQKPGKAPKPLIY | |
| | RANILVSGVPSRFSGSGSGQDYTFTISSLQPEDIATYYCLQFDAFPFTFG | |
| | CGTKLEIKGGGGSGGGGSGGGGSGGGGSEVQLVESGGGVVQPGGSL | |
| | RLSCAASGFTFNSFGMHWVRQAPGKCLEWVAFISSGSTSIYYANTVKG | |
| | RFTISRDNSKNTLYLQMNSLRAEDTAVYYCARSGYPTGGAMDYWGQ | |
| | GTSVTVSS | |
| | [SEQ ID NO: 75] | |
| Humanized | EVQLVESGGGVVQPGGSLRLSC | DIKMTQSPSSLSASVGDRVTITCKA |
| 9F11.B7 in | AASGFTFNSFGMHWVRQAPGK | SQDIYNYLSWFQQKPGKAPKPLIY |
| AB0194/ | GLEWVAFISSGSTSIYYANTVKG | RANILVSGVPSRFSGSGSGQDYTLT |
| AB0188 | RFTISRDNSKNTLYLQMNSLRA | ISSLQPEDIATYYCLQFDAFPFTFGS |
| (back | EDTAVYYCARDGYPTGGAMDY | GTKLEIK |
| mutations in | WGQGTSVTVSS | [SEQ ID NO: 84] |
| VH and VL | [SEQ ID NO: 29] | CDR1: KASQDIYNYLS [SEQ ID |
| underlined) | CDR1: GFTFNSF [SEQ ID NO: 59] | NO: 65] |
| | CDR2: SSGSTS [SEQ ID NO: 63] | CDR2: RANILVS [SEQ ID NO: 66] |
| | CDR3: DGYPTGGAMDY [SEQ | CDR3: LQFDAFPFT [SEQ ID NO: 67] |
| | ID NO: 54] | |
| scFv of | AB0194 (VH-VL): | |
| humanized | EVQLVESGGGVVQPGGSLRLSCAASGFTFNSFGMHWVRQAPGKCLE | |
| 9F11.B7 | WVAFISSGSTSIYYANTVKGRFTISRDNSKNTLYLQMNSLRAEDTAVY | |
| AB0194/ | YCARDGYPTGGAMDYWGQGTSVTVSSGGGGSGGGGSGGGGSGGGG | |
| AB0188 | SDIKMTQSPSSLSASVGDRVTITCKASQDIYNYLSWFQQKPGKAPKPLI | |
| | YRANILVSGVPSRFSGSGSGQDYTLTISSLQPEDIATYYCLQFDAFPFTF | |
| | GCGTKLEIK | |
| | [SEQ ID NO: 81] | |
| | AB0188 (VL-VH): | |
| | DIKMTQSPSSLSASVGDRVTITCKASQDIYNYLSWFQQKPGKAPKPLIY | |
| | RANILVSGVPSRFSGSGSGQDYTLTISSLQPEDIATYYCLQFDAFPFTFG | |
| | CGTKLEIKGGGGSGGGGSGGGGSGGGGSEVQLVESGGGVVQPGGSL | |
| | RLSCAASGFTFNSFGMHWVRQAPGKCLEWVAFISSGSTSIYYANTVKG | |
| | RFTISRDNSKNTLYLQMNSLRAEDTAVYYCARDGYPTGGAMDYWGQ | |
| | GTSVTVSS | |
| | [SEQ ID NO: 82] | |
| Humanized | EVQLVESGGGVVQPGGSLRLSC | DIKMTQSPSSLSASVGDRVTITCKA |
| 9F11.B7 in | AASGFTFNAFGMHWVRQAPGK | SQDIYNYLSWFQQKPGKAPKPLIY |
| AB0195/ | GLEWVAFISSGSTSIYYANTVKG | RANILVSGVPSRFSGSGSGQDYTLT |
| AB0189 | RFTISRDNSKNTLYLQMNSLRA | ISSLQPEDIATYYCLQFDAFPFTFGS |
| (back | EDTAVYYCARDGYPTGGAMDY | GTKLEIK |
| mutations in | WGQGTSVTVSS [SEQ ID NO: 14] | [SEQ ID NO: 84] |
| VH and VL | CDR1: GFTFNAF [SEQ ID NO: 62] | CDR1: KASQDIYNYLS [SEQ ID |
| underlined, | CDR2: SSGSTS [SEQ ID NO: 63] | NO: 65] |
| sequence | CDR3: DGYPTGGAMDY [SEQ | CDR2: RANILVS [SEQ ID NO: 66] |
| liability | ID NO: 54] | CDR3: LQFDAFPFT [SEQ ID NO: 67] |
| replacement | | |
| italicized) | | |
| scFv of | AB0195 (VH-VL): | |
| humanized | EVQLVESGGGVVQPGGSLRLSCAASGFTFNAFGMHWVRQAPGKCLE | |
| 9F11.B7 | WVAFISSGSTSIYYANTVKGRFTISRDNSKNTLYLQMNSLRAEDTAVY | |
| AB0195/ | YCARDGYPTGGAMDYWGQGTSVTVSSGGGGSGGGGSGGGGSGGGG | |
| AB0189 | SDIKMTQSPSSLSASVGDRVTITCKASQDIYNYLSWFQQKPGKAPKPLI | |
| | YRANILVSGVPSRFSGSGSGQDYTLTISSLQPEDIATYYCLQFDAFPFTF | |
| | GCGTKLEIK | |
| | [SEQ ID NO: 118] | |
| | AB0189 (VL-VH): | |
| | DIKMTQSPSSLSASVGDRVTITCKASQDIYNYLSWFQQKPGKAPKPLIY | |
| | RANILVSGVPSRFSGSGSGQDYTLTISSLQPEDIATYYCLQFDAFPFTFG | |
| | CGTKLEIKGGGGSGGGGSGGGGSGGGGSEVQLVESGGGVVQPGGSL | |
| | RLSCAASGFTFNAFGMHWVRQAPGKCLEWVAFISSGSTSIYYANTVK | |
| | GRFTISRDNSKNTLYLQMNSLRAEDTAVYYCARDGYPTGGAMDYWG | |
| | QGTSVTVSS | |
| | [SEQ ID NO: 119] | |
| Humanized | EVQLVESGGGVVQPGGSLRLSC | DIKMTQSPSSLSASVGDRVTITCKA |
| 9F11.B7 in | AASGFTFNSFGMHWVRQAPGK | SQDIYNYLSWFQQKPGKAPKPLIY |
| AB0196/ | GLEWVAFISSGSTSIYYANTVKG | RANILVSGVPSRFSGSGSGQDYTLT |
| AB0190 | RFTISRDNSKNTLYLQMNSLRA | ISSLQPEDIATYYCLQFDAFPFTFGS |
| (back | EDTAVYYCARSGYPTGGAMDY | GTKLEIK |
| mutations in | WGQGTSVTVSS | [SEQ ID NO: 84] |
| VH and VL | [SEQ ID NO: 76] | CDR1: KASQDIYNYLS [SEQ ID |
| underlined, | CDR1: GFTFNSF [SEQ ID NO: 59] | NO: 65] |
| sequence | CDR2: SSGSTS [SEQ ID NO: 63] | CDR2: RANILVS [SEQ ID NO: 66] |
| liability | CDR3: SGYPTGGAMDY [SEQ ID | CDR3: LQFDAFPFT [SEQ ID NO: 67] |
| replacement | NO: 79] | |
| italicized) | | |
| scFv of | AB0196 (VH-VL): | |
| humanized | EVQLVESGGGVVQPGGSLRLSCAASGFTFNSFGMHWVRQAPGKCLE | |
| 9F11.B7 | WVAFISSGSTSIYYANTVKGRFTISRDNSKNTLYLQMNSLRAEDTAVY | |
| AB0196/ | YCARSGYPTGGAMDYWGQGTSVTVSSGGGGSGGGGSGGGGSGGGG | |
| AB0190 | SDIKMTQSPSSLSASVGDRVTITCKASQDIYNYLSWFQQKPGKAPKPLI | |
| | YRANILVSGVPSRFSGSGSGQDYTLTISSLQPEDIATYYCLQFDAFPFTF | |
| | GCGTKLEIK | |
| | [SEQ ID NO: 120] | |
| | AB0190 (VL-VH): | |
| | DIKMTQSPSSLSASVGDRVTITCKASQDIYNYLSWFQQKPGKAPKPLIY | |
| | RANILVSGVPSRFSGSGSGQDYTLTISSLQPEDIATYYCLQFDAFPFTFG | |
| | CGTKLEIKGGGGSGGGGSGGGGSGGGGSEVQLVESGGGVVQPGGSL | |
| | RLSCAASGFTFNSFGMHWVRQAIGKCLEWVAFISSGSTSIYYANTVKG | |
| | RFTISRDNSKNTLYLQMNSLRAEDTAVYYCARSGYPTGGAMDYWGQ | |
| | GTSVTVSS | |
| | [SEQ ID NO: 121] | |
| Humanized | EVQLVESGGGVVQPGGSLRLSC | DIX₁MTQSPSSLSASVGDRVTITCK |
| 9F11.B7 | AASGFTFNX₁FGMHWVRQAPG | ASQDIYNYLSWFQQKPGKAPKPLI |
| consensus | KGLEWVAFISSGSTSIYYANTVK | YRANILVSGVPSRFSGSGSGQDYT |
| | GRFTISRDNSKNTLYLQMNSLR | X₂TISSLQPEDIATYYCLQFDAFPFT |
| | AEDTAVYYCARX₂GYPTGGAM | FGSGTKLEIK |
| | DYWGQGTSVTVSS | where X₁ is Q or K and |
| | where X₁ is S or A and | X₂ is F or L |
| | X₂ is D or S | [SEQ ID NO: 116] |
| | [SEQ ID NO: 115] | CDR1: KASQDIYNYLS [SEQ ID |
| | CDR1: GFTFNXF where X | NO: 65] |
| | is S or A | CDR2: RANILVS [SEQ ID NO: 66] |
| | [SEQ ID NO: 117] | CDR3: LQFDAFPFT [SEQ ID NO: 67] |
| | CDR2: SSGSTS [SEQ ID NO: 63] | |
| | CDR3: XGYPTGGAMDY where X | |
| | is D or S [SEQ ID NO: 112] | |
| 30A9.E9 | EVQLQESGPGLVQPSQSLSITCT | DIVMTQSPSSLAVTAGEKVTMRC |
| | VSGFSLTSFGVHWVRQSPGKGL | KSSQSLLWNVNQNNYLLWYQQK |
| | EWLGVIWSGGSTDSNAAFISRL | QGQPPKLLIYGASIRESWVPDRFT |
| | TITKDNSKSQVFFKMNSLQATD | GSGSGTDFTLTISNVHVEDLAVYY |
| | TAIYYCARSYFAMDYWGQGTS | CQHNHGSFLPYTFGGGTKLEIK |
| | VSVSS | [SEQ ID NO: 114] |
| | [SEQ ID NO: 113] | CDR1: KSSQSLLWNVNQNNYLL |
| | CDR1: GFSLTSF [SEQ ID NO: 87] | [SEQ ID NO: 106] |
| | CDR2: WSGGS [SEQ ID NO: 33] | CDR2: GASIRES [SEQ ID NO: 92] |
| | CDR3: SYFAMDY [SEQ ID | CDR3: QHNHGSFLPYT [SEQ ID |
| | NO: 89] | NO: 46] |
| 23A5.H8 | QVQLRQSGPGLVQPSQSLSITCT | GIVMTQSPSSLAVTAGEKVTMRC |
| | VSGFSLTSYGVHWVRQSPGKG | KSSQSLLWSVNQNNYLLWYQQK |
| | LEWLGVMWSGGSTDYNAAFM | QGQPPKLLIYGASIRQSWVPDRFT |
| | SRLSISKDNSKSQVFFTMNSLQ | GSGSGTDFTLSISNVHAEDLAVYY |
| | ADDTAIYYCARTHFGMDYWG | CQHNHGSFLPYTFGGGTKLEIK |
| | QGTPVTVSS | [SEQ ID NO: 109] |
| | [SEQ ID NO: 108] | CDR1: KSSQSLLWSVNQNNYLL |
| | CDR1: GFSLTSY [SEQ ID NO: 72] | [SEQ ID NO: 111] |
| | CDR2: WSGGS [SEQ ID NO: 33] | CDR2: GASIRQS [SEQ ID NO: 105] |
| | CDR3: THFGMDY [SEQ ID | CDR3: QHNHGSFLPYT [SEQ ID |
| | NO: 107] | NO: 46] |
| 20D6.H8 | EVQLQESGPGLVQPSQSLSITCT | GIVMTQSPSSLAVTAGEKVTMRC |
| | VSGFSLTSFGIHWVRQSPGKGL | KSSQSLLWNVNQNNYLVWYQQK |
| | EWLGVIWSGGNTDSNAAFISRL | QGQPPKLLIYGASIRESWVPDRFT |
| | SITKDISKSQVFFKMNSLQVTDT | GSGSGTDFTLTISNVHAEDLAVYY |
| | AIYYCARSYFAMDYWGQGTSV | CQHNHGSFLPYTFGGGTKLEIK |
| | TVSS | [SEQ ID NO: 103] |
| | [SEQ ID NO: 104] | CDR1: KSSQSLLWNVNQNNYLV |
| | CDR1: GFSLTSF [SEQ ID NO: 87] | [SEQ ID NO: 18] |
| | CDR2: WSGGN [SEQ ID NO: 102] | CDR2: GASIRES [SEQ ID NO: 92] |
| | CDR3: SYFAMDY [SEQ ID | CDR3: QHNHGSFLPYT [SEQ ID |
| | NO: 89] | NO: 46] |
| 15A10.G8 | EVQLQESGAELVRSGASIKLSC | EVLLTQSPAIIAASPGEKVTITCSA |
| | AASAFNIKDYFIHWVRQRPDQG | RSSVSYMSWYQQKPGSSPKIWIYG |
| | LEWIGWIDPENDDTEYAPKFQD | ISKLASGVPARFSGSGSGTYFSFTI |
| | KATMTADTSSNTAYLQLSSLTS | NNLEAEDVATYYCQQRSYYPFTF |
| | ADTAVYYCNALWSRGGYFDY | GSGTKLEIK |
| | WGQGTTLTVSS | [SEQ ID NO: 25] |
| | [SEQ ID NO: 22] | CDR1: SARSSVSYMS [SEQ ID |
| | CDR1: AFNIKDY [SEQ ID | NO: 53] |
| | NO: 26] | CDR2: GISKLAS [SEQ ID NO: 55] |
| | CDR2: DPENDD [SEQ ID NO: 37] | CDR3: QQRSYYPFT [SEQ ID |
| | CDR3: LWSRGGYFDY [SEQ ID | NO: 56] |
| | NO: 50] | |
| 13E1.A4 | EVQLQESGPELEKPGASVRISCK | SVLMTQTPLSLPVSLGDRASISCRS |
| | ASGYSFTAYNMNWVKQSNGKS | SQGIVHINGNTYLEWYLQKPGQSP |
| | LEWIGNIDPSYGDATYNQKFKG | KLLIYKVSNRFSGVPDRFSGSGSG |
| | KATLTVDKSSSTAYMQLKSLTS | TDFTLKISRVEAEDLGVYYCFQGS |
| | EDSAVYYCARDNYYGSGYFDY | HVPWTFGGGTKLEIK |
| | WGQGTTLTVSS | [SEQ ID NO: 58] |
| | [SEQ ID NO: 57] | CDR1: RSSQGIVHINGNTYLE [SEQ |
| | CDR1: GYSFTAY [SEQ ID | ID NO: 77] |
| | NO: 64] | CDR2: KVSNRFS [SEQ ID NO: 78] |
| | CDR2: DPSYGD [SEQ ID NO: 68] | CDR3: FQGSHVPWT [SEQ ID |
| | CDR3: DNYYGSGYFDY [SEQ ID | NO: 80] |
| | NO: 73] | |
| 12F8.H7 | EVQLQESGAELVRSGASVKLSC | GIVMTQAPLTLSVTIGQPASISCKS |
| | TVSGFNIKDYYMHWVKQRPEQ | SQSLLDSDGKTFLNWFLQRPGQSP |
| | GLEWIGWIDPENGDTENVPKFQ | KRLISLVSKLDSGVPDRFTGSGSGT |
| | GKATMTADTSSNTAYLQLRSLT | DFTLKLSRVEPEDLGVYYCWQGT |
| | SEDTAVYYCKSYYYDSSSRYV | HFPYTFGGGTKLEIK |
| | DVWGAGTTVTVSS | [SEQ ID NO: 85] |
| | [SEQ ID NO: 83] | CDR1: KSSQSLLDSDGKTFLN [SEQ |
| | CDR1: GFNIKDY [SEQ ID | ID NO: 90] |
| | NO: 86] | CDR2: LVSKLDS [SEQ ID NO: 91] |
| | CDR2: DPENGD [SEQ ID NO: 88] | CDR3: WQGTHFPYT [SEQ ID |
| | CDR3: YYYDSSSRYVDV [SEQ | NO: 93] |
| | ID NO: 127] | |
| 9E4.B7 | EVQLQESGAELMKPGASVKISC | GIVMTQSPASLSASVGETVTITCRA |
| | RTTGYTFSTYWIEWVKQRPGR | GENIHSYLAWYQQKQGKSPQLLV |
| | GPEWIGELFPGNSDTTLNEKFT | YNAKTLAEGVPSRFSGSGSGTQFS |
| | GKATFTADSSSNTAYMQLSSLT | LKINSLQPEDFGSYYCQHHYGTPR |
| | SEDSAVYYCARSGYYGSSLDY | TFGGGTKLEIK |
| | WGQGTTLTVSS | [SEQ ID NO: 95] |
| | [SEQ ID NO: 94] | CDR1: RAGENIHSYLA [SEQ ID |
| | CDR1: GYTFSTY [SEQ ID | NO: 99] |
| | NO: 96] | CDR2: NAKTLAE [SEQ ID NO: 100] |
| | CDR2: FPGNSD [SEQ ID NO: 97] | CDR3: QHHYGTPRT [SEQ ID |
| | CDR3: SGYYGSSLDY [SEQ ID | NO: 101] |
| | NO: 98] | |

In certain embodiments, the antigen-binding site of the present application comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VH of the same antibody disclosed in Table 1. In certain embodiments, the antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antibody discloses in Table 1. In certain embodiments, the antigen-binding site comprises the heavy chain CDR1, CDR2, and CDR3 and the light chain CDR1, CDR2, and CDR3 of an antibody disclosed in Table 1.

In certain embodiments, an antigen-binding site described in the present application is derived from 16B8.C8. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:1, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:2. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively.

In certain embodiments, an antigen-binding site described in the present application is derived from scFv-1292 or scFv-1301. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 3 or 12.

In certain embodiments, an antigen-binding site described in the present application is derived from scFv-1293 or scFv-1302. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:13, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 15 or 16.

In certain embodiments, an antigen-binding site described in the present application is derived from scFv-1294 or scFv-1303. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:110, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 19 or 20.

In certain embodiments, an antigen-binding site described in the present application is derived from scFv-1295 or scFv-1304. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:45, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 23 or 24.

In certain embodiments, an antigen-binding site described in the present application is derived from scFv-1296 or scFv-1305. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:122, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 27 or 28.

In certain embodiments, an antigen-binding site described in the present application is derived from scFv-1297 or scFv-1306. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:30. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 31 or 32.

In certain embodiments, an antigen-binding site described in the present application is derived from scFv-1298 or scFv-1307. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:34. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 35 or 36.

In certain embodiments, an antigen-binding site described in the present application is derived from scFv-1299 or scFv-1308. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:38. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 39 or 40.

In certain embodiments, an antigen-binding site described in the present application is derived from scFv-1300 or scFv-1309. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:41, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 43 or 44.

In certain embodiments, an antigen-binding site described in the present application is derived from scFv-1602 or scFv-2601. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:45, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:140. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 47 or 48. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 47.

In certain embodiments, an antigen-binding site described in the present application is derived from humanized 16B8.C8. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:49, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:17. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively.

In certain embodiments, an antigen-binding site described in the present application is derived from AB0305 or AB5030. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:128, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:129. In certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:147, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:148. In certain embodiments, the VH comprises CDR1, CDR2, and CDR3 comprising the amino acid sequences of SEQ ID NOs: 11, 130, and 131, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 130, and 131, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 132 or 133.

In certain embodiments, an antigen-binding site described in the present application is derived from AB0147 or AB7410. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:134, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:135. In certain embodiments, the VH comprises CDR1, CDR2, and CDR3 comprising the amino acid sequences of SEQ ID NOs: 11, 136, and 137, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 136, and 137, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 138 or 139.

In certain embodiments, an antigen-binding site described in the present application is derived from 9F11.B7. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:60, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:61. In certain embodiments, the VH comprises CDR1, CDR2, and CDR3 comprising the amino acid sequences of SEQ ID NOs: 59, 63, and 54, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 59, 63, and 54, respectively; and (b) a VL that comprises CDR1, CDR2, and CDR3 comprising the amino acid sequences of SEQ ID NOs: 65, 66, and 67, respectively.

In certain embodiments, an antigen-binding site described in the present application is derived from AB0191 or AB0185. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:29, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:69. In certain embodiments, the VH comprises CDR1, CDR2, and CDR3 comprising the amino acid sequences of SEQ ID NOs: 59, 63, and 54, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 59, 63, and 54, respectively; and (b) a VL that comprises CDR1, CDR2, and CDR3 comprising the amino acid sequences of SEQ ID NOs: 65, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 51 or 52.

In certain embodiments, an antigen-binding site described in the present application is derived from AB0192 or AB0186. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:14, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:69. In certain embodiments, the VH comprises CDR1, CDR2, and CDR3 comprising the amino acid sequences of SEQ ID NOs: 62, 63, and 54, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 62, 63, and 54, respectively; and (b) a VL that comprises CDR1, CDR2, and CDR3 comprising the amino acid sequences of SEQ ID NOs: 65, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 70 or 71.

In certain embodiments, an antigen-binding site described in the present application is derived from AB0193 or AB0187. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:76, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:69. In certain embodiments, the VH comprises CDR1, CDR2, and CDR3 comprising the amino acid sequences of SEQ ID NOs: 59, 63, and 79, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 59, 63, and 79, respectively; and (b) a VL that comprises CDR1, CDR2, and CDR3 comprising the amino acid sequences of SEQ ID NOs: 65, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 74 or 75.

In certain embodiments, an antigen-binding site described in the present application is derived from AB0194 or AB0188. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:29, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:84. In certain embodiments, the VH comprises CDR1, CDR2, and CDR3 comprising the amino acid sequences of SEQ ID NOs: 59, 63, and 54, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 59, 63, and 54, respectively; and (b) a VL that comprises CDR1, CDR2, and CDR3 comprising the amino acid sequences of SEQ ID NOs: 65, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 81 or 82.

In certain embodiments, an antigen-binding site described in the present application is derived from AB0195 or AB0189. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:14, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:84. In certain embodiments, the VH comprises CDR1, CDR2, and CDR3 comprising the amino acid sequences of SEQ ID NOs: 62, 63, and 54, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 62, 63, and 54, respectively; and (b) a VL that comprises CDR1, CDR2, and CDR3 comprising the amino acid sequences of SEQ ID NOs: 65, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 118 or 119.

In certain embodiments, an antigen-binding site described in the present application is derived from AB0196 or AB0190. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:76, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:84. In certain embodiments, the VH comprises CDR1, CDR2, and CDR3 comprising the amino acid sequences of SEQ ID NOs: 59, 63, and 79, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 59, 63, and 79, respectively; and (b) a VL that comprises CDR1, CDR2, and CDR3 comprising the amino acid sequences of SEQ ID NOs: 65, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 120 or 121.

In certain embodiments, an antigen-binding site described in the present application is derived from humanized 9F11.B7. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:115, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:116. In certain embodiments, the VH comprises CDR1, CDR2, and CDR3 comprising the amino acid sequences of SEQ ID NOs: 117, 63, and 112, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 117, 63, and 112, respectively; and (b) a VL that comprises CDR1, CDR2, and CDR3 comprising the amino acid sequences of SEQ ID NOs: 65, 66, and 67, respectively.

In certain embodiments, an antigen-binding site described in the present application is derived from 30A9.E9. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:113, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:114. In certain embodiments, the VH comprises CDR1, CDR2, and CDR3 comprising the amino acid sequences of SEQ ID NOs: 87, 33, and 89, respectively. In certain embodiments, the VL comprises CDR1, CDR2, and CDR3 comprising the amino acid sequences of SEQ ID NOs: 106, 92, and 46, respectively. In certain embodiments, the antigen-binding site comprises (a) a VH that comprises CDR1, CDR2, and CDR3 comprising the amino acid sequences of SEQ ID NOs: 87, 33, and 89, respectively; and (b) a VL that comprises CDR1, CDR2, and CDR3 comprising the amino acid sequences of SEQ ID NOs: 106, 92, and 46, respectively.

In certain embodiments, an antigen-binding site described in the present application is derived from 23A5.H8. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:108, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:109. In certain embodiments, the VH comprises CDR1, CDR2, and CDR3 comprising the amino acid sequences of SEQ ID NOs: 72, 33, and 107, respectively. In certain embodiments, the VL comprises CDR1, CDR2, and CDR3 comprising the amino acid sequences of SEQ ID NOs: 111, 105, and 46, respectively. In certain embodiments, the antigen-binding site comprises (a) a VH that comprises CDR1, CDR2, and CDR3 comprising the amino acid sequences of SEQ ID NOs: 72, 33, and 107, respectively; and (b) a VL that comprises CDR1, CDR2, and CDR3 comprising the amino acid sequences of SEQ ID NOs: 111, 105, and 46, respectively.

In certain embodiments, an antigen-binding site described in the present application is derived from 20D6.H8. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:104, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:103. In certain embodiments, the VH comprises CDR1, CDR2, and CDR3 comprising the amino acid sequences of SEQ ID NOs: 87, 102, and 89, respectively. In certain embodiments, the VL comprises CDR1, CDR2, and CDR3 comprising the amino acid sequences of SEQ ID NOs: 18, 92, and 46, respectively. In certain embodiments, the antigen-binding site comprises (a) a VH that comprises CDR1, CDR2, and CDR3 comprising the amino acid sequences of SEQ ID NOs: 87, 102, and 89, respectively; and (b) a VL that comprises CDR1, CDR2, and CDR3 comprising the amino acid sequences of SEQ ID NOs: 18, 92, and 46, respectively.

In certain embodiments, an antigen-binding site described in the present application is derived from 15A10.G8. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:22, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:25. In certain embodiments, the VH comprises CDR1, CDR2, and CDR3 comprising the amino acid sequences of SEQ ID NOs: 26, 37, and 50, respectively. In certain embodiments, the VL comprises CDR1, CDR2, and CDR3 comprising the amino acid sequences of SEQ ID NOs: 53, 55, and 56, respectively. In certain embodiments, the antigen-binding site comprises (a) a VH that comprises CDR1, CDR2, and CDR3 comprising the amino acid sequences of SEQ ID NOs: 26, 37, and 50, respectively; and (b) a VL that comprises CDR1, CDR2, and CDR3 comprising the amino acid sequences of SEQ ID NOs: 53, 55, and 56, respectively.

In certain embodiments, an antigen-binding site described in the present application is derived from 13E1.A4. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:57, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:58. In certain embodiments, the VH comprises CDR1, CDR2, and CDR3 comprising the amino acid sequences of SEQ ID NOs: 64, 68, and 73, respectively. In certain embodiments, the VL comprises CDR1, CDR2, and CDR3 comprising the amino acid sequences of SEQ ID NOs: 77, 78, and 80, respectively. In certain embodiments, the antigen-binding site comprises (a) a VH that comprises CDR1, CDR2, and CDR3 comprising the amino acid sequences of SEQ ID NOs: 64, 68, and 73, respectively; and (b) a VL that comprises CDR1, CDR2, and CDR3 comprising the amino acid sequences of SEQ ID NOs: 77, 78, and 80, respectively.

In certain embodiments, an antigen-binding site described in the present application is derived from 12F8.H7. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:83, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 86, 88, and 127, respectively. In certain embodiments, the VL comprises CDR1, CDR2, and CDR3 comprising the amino acid sequences of SEQ ID NOs: 90, 91, and 93, respectively. In certain embodiments, the antigen-binding site comprises (a) a VH that comprises CDR1, CDR2, and CDR3 comprising the amino acid sequences of SEQ ID NOs: 86, 88, and 127, respectively; and (b) a VL that comprises CDR1, CDR2, and CDR3 comprising the amino acid sequences of SEQ ID NOs: 90, 91, and 93, respectively.

In certain embodiments, an antigen-binding site described in the present application is derived from 9E4.B7. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:94, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:95. In certain embodiments, the VH comprises CDR1, CDR2, and CDR3 comprising the amino acid sequences of SEQ ID NOs: 96, 97, 98, respectively. In certain embodiments, the VL comprises CDR1, CDR2, and CDR3 comprising the amino acid sequences of SEQ ID NOs: 99, 100, 101, respectively. In certain embodiments, the antigen-binding site comprises (a) a VH that comprises CDR1, CDR2, and CDR3 comprising the amino acid sequences of SEQ ID NOs: 96, 97, 98, respectively; and (b) a VL that comprises CDR1, CDR2, and CDR3 comprising the amino acid sequences of SEQ ID NOs: 99, 100, 101, respectively.

In each of the foregoing embodiments, it is contemplated herein that the VH and/or VL sequences that together bind CLEC12A may contain amino acid alterations (e.g., at least 1, 2, 3, 4, 5, or 10 amino acid substitutions, deletions, or additions) in the framework regions of the VH and/or VL without affecting their ability to bind to CLEC12A significantly.

In certain embodiments, an antigen-binding site described in the present application binds human CLEC12A with a K_{D} (i.e., dissociation constant) of 1 nM or lower, 5 nM or lower, 10 nM or lower, 15 nM or lower, or 20 nM or lower, as measured by surface plasmon resonance (SPR) (e.g., using the method described in Example 1 infra) or by bio-layer interferometry (BLI), and/or binds CLEC12A from a body fluid, tissue, and/or cell of a subject. In certain embodiments, an antigen-binding site described in the present application has a K_{d} (i.e., off-rate, also called K_{off}) equal to or lower than 1×10⁻⁵, 1×10⁻⁴, 1×10⁻³, 5×10⁻³, 0.01, 0.02, or 0.05 l/s, as measured by SPR (e.g., using the method described in Example 1 infra) or by BLI.

In certain embodiments, an antigen-binding site derived from 15A10.G8 or 20D6.A8 binds cynomolgus CLEC12A with a K_{D} (i.e., dissociation constant) of 5 nM or lower, 10 nM or lower, 15 nM or lower, 20 nM or lower, or 30 nM or lower, as measured by surface plasmon resonance (SPR) (e.g., using the method described in Example 1 infra) or by bio-layer interferometry (BLI), and/or binds CLEC12A from a body fluid, tissue, and/or cell of a subject. In certain embodiments, an antigen-binding site described in the present application has a K_{d} (i.e., off-rate, also called K_{off}) equal to or lower than 1×10⁻³, 5×10⁻³, 0.01, 0.02, or 0.03 l/s, as measured by SPR (e.g., using the method described in Example 1 infra) or by BLI.

Variations in the glycosylation status of CLEC12A on the surface of different cell types has been reported (Marshall et al, (2006) Eur J Immunol. 36(8):2159-69). CLEC12A expressed on the surface of AML cells from different patients may have different glycosylation patterns as well. Moreover, branched glycans can restrict accessibility to the protein component of CLEC12A, limiting diversity of available epitopes. Certain antigen-binding sites described in the present application can overcome the glycosylation variations. In certain embodiments, an antigen-binding site described in the present application, e.g., an antigen-binding site derived from 16B8.C8, a humanized 16B8.C8, 9F11.B7, or a humanized 9F11.B7, binds CLEC12A (e.g., human CLEC12A) in a glycosylation independent manner, i.e., binds both a glycosylated CLEC12A and a de-glycosylated CLEC12A. In certain embodiments, the ratio of the K_{D} at which the antigen-binding site binds deglycosylated CLEC12A to the K_{D} at which the antigen-binding site binds glycosylated CLEC12A is within the range of 1:10 to 10:1 (e.g., within the range of 1:5 to 5:1, 1:3 to 3:1, or 1:2 to 2:1). In certain embodiments, the ratio is about 1:5, about 1:3, about 1:2, about 1:1.5, about 1:1, about 1.5:1, about 2:1, about 3:1, or about 5:1. In certain embodiments, the ratio is about 1:1. In another aspect, the instant disclosure provides an antigen-binding site that binds CLEC12A (e.g., human CLEC12A) in a glycosylation independent manner.

CLEC12A containing a K244Q substitution is a polymorphic variant prevalent in about 30% of the human population. In some embodiments, the antigen-binding site disclosed herein binds CLEC12A-K244Q. In certain embodiments, the ratio of the K_{D} at which the antigen-binding site binds CLEC12A-K244Q to the K_{D} at which the antigen-binding site binds wild-type CLEC12A is within the range of 1:2 to 2:1. In certain embodiments, the ratio is about 1:2, about 1:1.5, about 1:1, about 1.5:1, or about 2:1. In certain embodiments, the ratio is about 1:1.

In another aspect, the present application provides an antigen-binding site that competes for binding to CLEC12A (e.g., human CLEC12A) with an antigen-binding site described above. In certain embodiments, an antigen-binding site described in the present application competes with an antigen-binding site derived from 16B8.C8 disclosed above for binding to CLEC12A. In one embodiment, the antigen-binding site competes with 16B8.C8 for binding to CLEC12A. In certain embodiments, the antigen-binding site of the present application competes with an antigen-binding site derived from a humanized 16B8.C8 disclosed above for binding to CLEC12A. In one embodiment, the antigen-binding site competes with a humanized 16B8.C8 for binding to CLEC12A. In certain embodiments, the antigen-binding site described in the present application competes with an antigen-binding site derived from 9F11.B7 disclosed above for binding to CLEC12A. In one embodiment, the antigen-binding site competes with 9F11.B7 for binding to CLEC12A. In certain embodiments, an antigen-binding site described in the present application competes with an antigen-binding site derived from a humanized 9F11.B7 disclosed above for binding to CLEC12A. In one embodiment, an antigen-binding site competes with a humanized 9F11.B7 for binding to CLEC12A. In certain embodiments, an antigen-binding site described in the present application competes with an antigen-binding site derived from 12F8.H7, 13E1.A4, 15A10.E8, 20D6.H8, or 23A5.H8 disclosed above for binding to CLEC12A. In some embodiments, the antigen-binding site competes with 12F8.H7, 13E1.A4, 15A10.E8, 20D6.H8, or 23A5.H8 for binding to CLEC12A.

Proteins with Antigen-Binding Sites

An antigen-binding site disclosed herein can be present in an antibody or antigen-binding fragment thereof. The antibody can be a monoclonal antibody, a chimeric antibody, a diabody, a Fab fragment, a Fab′ fragment, or F(ab′)2 fragment, an Fv, a bispecific antibody, a bispecific Fab2, a bispecific (mab)2, a humanized antibody, an artificially-generated human antibody, bispecific T-cell engager, bispecific NK cell engager, a single chain antibody (e.g., single-chain Fv fragment or scFv), triomab, knobs-into-holes (kih) IgG with common light chain, crossmab, ortho-Fab IgG, DVD-Ig, 2 in 1-IgG, IgG-scFv, sdFv2-Fc, bi-nanobody, tandAb, dual-affinity retargeting antibody (DART), DART-Fc, scFv-HSA-scFv (where HSA=human serum albumin), or dock-and-lock (DNL)-Fab3.

In certain embodiments, an antigen-binding site disclosed herein is linked to an amino acid sequence at least 90% (e.g., 90%, 91%, 92%, 93%, 94%, 95%, 96%, 97%, 98%, 99%, or 100%) identical to an antibody constant region, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, an antigen-binding site disclosed herein can be linked to a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the antigen-binding site is linked to an IgG constant region including hinge, CH2 and CH3 domains with or without a CH1 domain. In some embodiments, the amino acid sequence of the constant region is at least 90% (e.g., 90%, 91%, 92%, 93%, 94%, 95%, 96%, 97%, 98%, 99%, or 100%) identical to a human antibody constant region, such as an human IgG1 constant region, a human IgG2 constant region, a human IgG3 constant region, or a human IgG4 constant region. In one embodiment, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence DKTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO:21). In some other embodiments, the amino acid sequence of the constant region is at least 90% (e.g., at least 91%, at least 92%, at least 93%, at least 94%, at least 95%, at least 96%, at least 97%, at least 98%, at least 99%, or 100%) identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, the antigen-binding site is linked to a portion of an antibody Fc domain sufficient to bind CD16. Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

In some embodiments, the antibody constant domain comprises a CH2 domain and a CH3 domain of an IgG antibody, for example, a human IgG1 antibody. In some embodiments, mutations are introduced in the antibody constant domain to enable heterdimerization with another antibody constant domain. For example, if the antibody constant domain is derived from the constant domain of a human IgG1, the antibody constant domain can comprise an amino acid sequence at least 90% (e.g., at least 91%, at least 92%, at least 93%, at least 94%, at least 95%, at least 96%, at least 97%, at least 98%, at least 99%, or 100%) identical to amino acids 234-332 of a human IgG1 antibody, and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

To facilitate formation of an asymmetric protein, Fc domain heterodimerization is contemplated. Mutations (e.g., amino acid substitutions) in the Fc domain that promote heterodimerization are described, for example, in International Application Publication No. WO2019157366, which is not incorporated herein by reference.

The proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third and fourth expression vectors can be stably transfected together into host cells to produce the multimeric proteins.

To achieve the highest yield of the proteins, different ratios of the first, second, third and fourth expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of a protein comprising an antigen-binding site disclosed herein. The protein can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

Accordingly, in another aspect, the present application provides one or more isolated nucleic acids comprising sequences encoding an immunoglobulin heavy chain and/or immunoglobulin light chain variable region of any one of the foregoing antibodies. The application provides one or more expression vectors that express the immunoglobulin heavy chain and/or immunoglobulin light chain variable region of any one of the foregoing antibodies. Similarly the application provides host cells comprising one or more of the foregoing expression vectors and/or isolated nucleic acids.

In certain embodiments, the antibody binds CLEC12A with a K_{D} of 25 nM, 20 nM, 15 nM, 10 nM, 9 nM, 8 nM, 7 nM, 6 nM, 5 nM, 4 nM, 3 nM, 2 nM, 1 nM, 0.1 nM or lower, as measured using standard binding assays, for example, surface plasmon resonance or bio-layer interferometry. In certain embodiments the antibody binds CLEC12A from a body fluid, tissue and/or cell of a subject.

Competition assays for determining whether an antibody binds to the same epitope as, or competes for binding with a disclosed antibody are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen (e.g., a human CLEC12A protein or fragment thereof) bound to a solid surface or expressed on a cell surface, a test CLEC12A-binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

### CAR T Cells, CLEC12A/CD3-Directed Bispecific T-Cell Engagers, Immunocytokines, Antibody-Drug Conjugates, and Immunotoxins

Another aspect of the present application provides a molecule or complex comprising an antigen-binding site that binds CLEC12A as disclosed herein. Exemplary molecules or complexes include but are not limited to chimeric antigen receptors (CARs), T-cell engagers (e.g., CLEC12A/CD3-directed bispecific T-cell engagers), immunocytokines, antibody-drug conjugates, and immunotoxins.

Any antigen-binding site that binds CLEC12A as disclosed herein can be used. In certain embodiments, the VH, VL, and/or CDR sequences of the antigen-binding site that binds CLEC12A are provided in Table 1. In certain embodiments, the antigen-binding site that binds CLEC12A is an scFv. In certain embodiments, the scFv comprises an amino acid sequence at least 90% (e.g., at least 91%, at least 92%, at least 93%, at least 94%, at least 95%, at least 96%, at least 97%, at least 98%, or at least 99%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, 82, 118, 119, 120, 121, 132, 133, 138, and 139. In certain embodiments, the scFv comprises an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, 82, 118, 119, 120, 121, 132, 133, 138, and 139.

In certain embodiments, the antigen-binding site that binds CLEC12A in the molecule or complex (e.g., CAR, T-cell engager, immunocytokine, antibody-drug conjugate, or immunotoxin) comprises a heavy chain variable domain comprising CDR1, CDR2, and CDR3 sequences represented by the amino acid sequences of SEQ ID NOs: 11, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:45; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:140. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:47 or SEQ ID NO:48. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:47.

### Chimeric Antigen Receptors (CARs)

In certain embodiments, the present application provides a CLEC12A-targeting CAR comprising an antigen-binding site that binds CLEC12A as disclosed herein (see, e.g., Table 1). The CLEC12A-targeting CAR can comprise an Fab fragment or an scFv.

The term “chimeric antigen receptor” or alternatively a “CAR” refers to a recombinant polypeptide construct comprising at least an extracellular antigen binding domain, a transmembrane domain and an intracellular signaling domain comprising a functional signaling domain derived from a stimulatory molecule (also referred to herein as a “primary signaling domain”).

Accordingly, in certain embodiments, the CAR comprises an extracellular antigen-binding site that binds CLEC12A as disclosed herein, a transmembrane domain, and an intracellular signaling domain comprising a primary signaling domain. In certain embodiments, the CAR further comprises one or more functional signaling domains derived from at least one costimulatory molecule (also referred to as a “costimulatory signaling domain”).

In certain embodiments, the CAR comprises a chimeric fusion protein comprising an antigen-binding site that binds CLEC12A (e.g., CLEC12A-binding scFv) disclosed herein as an extracellular antigen binding domain, a transmembrane domain, and an intracellular signaling domain comprising a primary signaling domain. In certain embodiments, the CAR comprises a chimeric fusion protein comprising an antigen-binding site that binds CLEC12A (e.g., CLEC12A-binding scFv) disclosed herein as an extracellular antigen binding domain, a transmembrane domain, and an intracellular signaling domain comprising a costimulatory signaling domain and a primary signaling domain. In certain embodiments, the CAR comprises a chimeric fusion protein comprising an antigen-binding site that binds CLEC12A (e.g., CLEC12A-binding scFv) disclosed herein as an extracellular antigen binding domain, a transmembrane domain, and an intracellular signaling domain comprising two costimulatory signaling domains and a primary signaling domain. In certain embodiments, the CAR comprises a chimeric fusion protein comprising an antigen-binding site that binds CLEC12A (e.g., CLEC12A-binding scFv) disclosed herein as an extracellular antigen binding domain, a transmembrane domain, and an intracellular signaling domain comprising at least two costimulatory signaling domains and a primary signaling domain.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 11, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:45; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:140. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:47 or SEQ ID NO:48. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:47.

With respect to the transmembrane domain, in various embodiments, the CAR is designed to comprise a transmembrane domain that is fused to the extracellular domain of the CAR. In one embodiment, the transmembrane domain is one that naturally is associated with one of the domains in the CAR. In some instances, the transmembrane domain can be selected or modified by amino acid substitution to avoid binding of such domains to the transmembrane domains of the same or different surface membrane proteins to minimize interactions with other members of the receptor complex. In another embodiment, the transmembrane domain is capable of homodimerization with another CAR on the CAR T cell surface. In another embodiment, the amino acid sequence of the transmembrane domain may be modified or substituted so as to minimize interactions with the binding domains of the native binding partner present in the same CAR T cell.

The transmembrane domain may be derived from any naturally occurring membrane-bound or transmembrane protein. In one embodiment, the transmembrane region is capable of signaling to the intracellular domain(s) whenever the CAR has bound to a target. In some embodiments, the transmembrane domain comprises the transmembrane region(s) of one or more proteins selected from the group consisting of TCR α chain, TCR β chain, TCR ζ chain, CD28, CD3c, CD45, CD4, CD5, CD8, CD9, CD16, CD22, CLEC12A, CD37, CD64, CD80, CD86, CD134, CD137, and CD154. In some embodiments, the transmembrane domain comprises the transmembrane region(s) of one or more proteins selected from the group consisting of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C.

The extracellular CLEC12A-binding domain (e.g., CLEC12A-binding scFv domain) domain can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G₄S)₄ linker, a KIR2DS2 hinge, and a CD8α hinge.

The intracellular signaling domain of the CAR described in the present application is responsible for activation of at least one of the specialized functions of the immune cell (e.g., cytolytic activity or helper activity, including the secretion of cytokines, of a T cell) in which the CAR has been placed in. Thus, as used herein, the term “intracellular signaling domain” refers to the portion of a protein which transduces an effector function signal and directs the cell to perform a specialized function. Although usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain of the CAR comprises a primary signaling domain (i.e., a functional signaling domain derived from a stimulatory molecule) and one or more costimulatory signaling domains (i.e., functional signaling domains derived from at least one costimulatory molecule).

As used herein, the term “stimulatory molecule” refers to a molecule expressed by an immune cell, e.g., a T cell, an NK cell, or a B cell, that provide the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an MHC molecule loaded with a peptide, and which leads to mediation of a T cell response, including, but not limited to, proliferation, activation, differentiation, and the like.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the present application include those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In one embodiment, the primary signaling domain in any one or more CARs described in the present application comprises a cytoplasmic signaling sequence derived from CD3-zeta.

In some embodiments, the primary signaling domain is a functional signaling domain of TCR zeta, FcR gamma, FcR beta, CD3 gamma, CD3 delta, CD3 epsilon, CD5, CD22, CD79a, CD79b, CD66d, 4-1BB, and/or CD3-zeta. In an embodiment, the intracellular signaling domain comprises a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and/or DAP12. In a particular embodiment, the primary signaling domain is a functional signaling domain of the zeta chain associated with the T cell receptor complex.

As used herein, the term “costimulatory molecule” refers to a cognate binding partner on a T cell that specifically binds with a costimulatory ligand, thereby mediating a costimulatory response by the T cell, such as, but not limited to, proliferation. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1, CD11a/CD18), CD2, CD7, CD258 (LIGHT), NKG2C, B7-H3, and a ligand that specifically binds with CD83, and the like. Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and a ligand that specifically binds with CD83. In some embodiments, the costimulatory signaling domain of the CAR is a functional signaling domain of a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

As used herein, the term “signaling domain” refers to the functional portion of a protein which acts by transmitting information within the cell to regulate cellular activity via defined signaling pathways by generating second messengers or functioning as effectors by responding to such messengers.

The cytoplasmic signaling sequences within the cytoplasmic signaling portion of the CAR described in the present application may be linked to each other in a random or specified order. Optionally, a short oligo- or polypeptide linker, for example, between 2 and 10 amino acids in length may form the linkage.

Another aspect of the present application provides a nucleic acid encoding a CLEC12A-targeting CAR disclosed herein. The nucleic acid is useful for expressing the CAR in an effector cell (e.g., T cell) by introducing the nucleic acid to the cell.

Modifications may be made in the sequence to create an equivalent or improved variant, for example, by changing one or more of the codons according to the codon degeneracy table. A DNA codon degeneracy table is provided in Table 2.

TABLE 2
Amino Acid Codons

| Amino Acids | One | Three | |
|---|---|---|---|
| | letter | letter | |
| | code | code | Codons |
| Alanine | A | Ala | GCA GCC GCG GCU |
| Cysteine | C | Cys | UGC UGU |
| Aspartic acid | D | Asp | GAC GAU |
| Glutamic acid | E | Glu | GAA GAG |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC |
| | | | CUG CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC |
| | | | CGG CGU |
| Serine | S | Ser | AGC AGU UCA UCC |
| | | | UCG UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

In certain embodiments, the nucleic acid is a DNA molecule (e.g., a cDNA molecule). In certain embodiments, the nucleic acid further comprises an expression control sequence (e.g., promoter and/or enhancer) operably linked to the CAR coding sequence. In certain embodiments, the present application provides a vector comprising the nucleic acid. The vector can be a viral vector (e.g., AAV vector, lentiviral vector, or adenoviral vector) or a non-viral vector (e.g., plasmid).

In certain embodiments, the nucleic acid is an RNA molecule (e.g., an mRNA molecule). A method for generating mRNA for use in transfection can involve in vitro transcription of a template with specially designed primers, followed by polyA addition, to produce an RNA construct containing 3′ and 5′ untranslated sequences, a 5′ cap and/or Internal Ribosome Entry Site (IRES), the nucleic acid to be expressed, and a polyA tail, typically 50-2000 bases in length. The RNA molecule can be further modified to increase translational efficiency and/or stability, e.g., as disclosed in U.S. Pat. Nos. 8,278,036; 8,883,506, and 8,716,465. RNA molecules so produced can efficiently transfect different kinds of cells.

In one embodiment, the nucleic acid encodes an amino acid sequence comprising a signal peptide at the amino-terminus of the CAR. Such signal peptide can facilitate the cell surface localization of the CAR when it is expressed in an effector cell, and is cleaved from the CAR during cellular processing. In one embodiment, the nucleic acid encodes an amino acid sequence comprising a signal peptide at the N-terminus of the extracellular CLEC12A-binding domain (e.g., CLEC12A-binding scFv domain).

RNA or DNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)).

In another aspect, the present application provides an immune effector cell expressing the CLEC12A-targeting CAR. Also provided is an immune effector cell comprising the nucleic acid encoding the CLEC12A-targeting CAR. The immune effector cells include but are not limited to T cells and NK cells. In certain embodiments, the T cell is selected from a CD8⁺ T cell, a CD4⁺ T cell, and an NKT cell. The T cell or NK cell can be a primary cell or a cell line.

The immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors, by methods known in the art. The immune effector cells can also be differentiated in vitro from a pluripotent or multipotent cell (e.g., a hematopoietic stem cell). In some embodiments, the present application provides a pluripotent or multipotent cell (e.g., a hematopoietic stem cell) expressing the CLEC12A-targeting CAR (e.g., expressing the CAR on the plasma membrane) or comprising a nucleic acid disclosed herein.

In certain embodiments, the immune effector cells are isolated and/or purified. For example, regulatory T cells can be removed from a T cell population using a CD25-binding ligand. Effector cells expressing a checkpoint protein (e.g., PD-1, LAG-3, or TIM-3) can be removed by similar methods. In certain embodiments, the effector cells are isolated by a positive selection step. For example, a population of T cells can be isolated by incubation with anti-CD3/anti-CD28-conjugated beads. Other cell surface markers, such as IFN-7, TNF-α, IL-17A, IL-2, IL-3, IL-4, GM-CSF, IL-10, IL-13, granzyme B, and perforin, can also be used for positive selection.

Immune effector cells may be activated and expanded generally using methods known in the art, e.g., as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publications Nos. 2006/0121005 and 2016/0340406. For example, in certain embodiments, T cells can be expanded and/or activated by contact with an anti-CD3 antibody and an anti-CD28 antibody, under conditions appropriate for stimulating proliferation of the T cells. The cells can be expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. Multiple cycles of stimulation may be desirable for prolonged cell culture (e.g., culture for a period of 60 days or more). In certain embodiments, the cell culture comprises serum (e.g., fetal bovine or human serum), interleukin-2 (IL-2), insulin, IFN-γ, IL-4, IL-7, GM-CSF, IL-10, IL-12, IL-15, TGFβ, TNF-α, or a combination thereof. Other additives for the growth of cells known to the skilled person, e.g., surfactant, plasmanate, and reducing agents such as N-acetyl-cysteine and 2-mercaptoethanol, can also be included in the cell culture. In certain embodiments, the immune effector cell of the present application is a cell obtained from in vitro expansion.

Further embodiments of the CLEC12A-targeting CAR (e.g., regulatable CAR), nucleic acid encoding the CAR, and effector cells expressing the CAR or comprising the nucleic acid are provided in U.S. Pat. Nos. 7,446,190 and 9,181,527, U.S. Patent Application Publication Nos. 2016/0340406 and 2017/0049819, and International Patent Application Publication No. WO2018/140725.

### CLEC12A/CD3-Directed Bispecific T-Cell Engagers

In certain embodiments, the present application provides a CLEC12A/CD3-directed bispecific T-cell engager comprising an antigen-binding site that binds CLEC12A disclosed herein. In certain embodiments, the CLEC12A/CD3-directed bispecific T-cell engager comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, 82, 118, 119, 120, 121, 132, 133, 138, and 139. In certain embodiments, the cytokine is connected to the Fc domain directly or via a linker.

In certain embodiments, the CLEC12A/CD3-directed bispecific T-cell engager further comprises an antigen-binding site that binds CD3. Exemplary antigen-binding sites that bind CD3 are disclosed in International Patent Application Publication Nos. WO2014/051433 and WO2017/097723.

Another aspect of the present application provides a nucleic acid encoding at least one polypeptide of the CLEC12A/CD3-directed bispecific T-cell engager, wherein the polypeptide comprises an antigen-binding site that binds CLEC12A. In certain embodiments, the nucleic acid further comprises a nucleotide sequence encoding a signal peptide that, when expressed, is at the N-terminus of one or more of the polypeptides of the CLEC12A/CD3-directed bispecific T-cell engager. Also provided is a vector (e.g., a viral vector) comprising the nucleic acid, a producer cell comprising the nucleic acid or vector, and a producer cell expressing the CLEC12A/CD3-directed bispecific T-cell engager.

### Immunocytokines

In certain embodiments, the present application provides an immunocytokine comprising an antigen-binding site that binds CLEC12A disclosed herein and a cytokine. Any cytokine (e.g., pro-inflammatory cytokines) known in the art can be used, including but not limited to IL-2, IL-4, IL-10, IL-12, IL-15, TNF, IFNα, IFNγ, and GM-CSF. More exemplary cytokines are disclosed in U.S. Pat. No. 9,567,399. In certain embodiments, the antigen-binding site is connected to the cytokine by chemical conjugation (e.g., covalent or noncovalent chemical conjugation). In certain embodiments, the antigen-binding site is connected to the cytokine by fusion of polypeptide. The immunocytokine can further comprise an Fc domain connected to the antigen-binding site that binds CLEC12A. In certain embodiments, the immunocytokine comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, 82, 118, 119, 120, 121, 132, 133, 138, and 139. In certain embodiments, the cytokine is connected to the Fc domain directly or via a linker.

In another aspect, the present application provides a nucleic acid encoding at least one polypeptide of the immunocytokine, wherein the polypeptide comprises an antigen-binding site that binds CLEC12A. In certain embodiments, the nucleic acid further comprises a nucleotide sequence encoding a signal peptide that, when expressed, is at the N-terminus of one or more of the polypeptides of the immunocytokine. Also provided is a vector (e.g., a viral vector) comprising the nucleic acid, a producer cell comprising the nucleic acid or vector, and a producer cell expressing the immunocytokine.

### Antibody-Drug Conjugates

In certain embodiments, the present application provides an antibody-drug conjugate comprising an antigen-binding site that binds CLEC12A disclosed herein and a cytotoxic drug moiety. Exemplary cytotoxic drug moieties are disclosed in International Patent Application Publication Nos. WO2014/160160 and WO2015/143382. In certain embodiments, the cytotoxic drug moiety is selected from auristatin, N-acetyl-γ calicheamicin, maytansinoid, pyrrolobenzodiazepine, and SN-38. The antigen-binding site can be connected to the cytotoxic drug moiety by chemical conjugation (e.g., covalent or noncovalent chemical conjugation). In certain embodiments, the antibody-drug conjugate further comprises an Fc domain connected to the antigen-binding site that binds CLEC12A. In certain embodiments, the antibody-drug conjugate comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, 82, 118, 119, 120, 121, 132, 133, 138, and 139. In certain embodiments, the cytotoxic drug moiety is connected to the Fc domain directly or via a linker.

### Immunotoxins

In certain embodiments, the present application provides an immunotoxin comprising an antigen-binding site that binds CLEC12A disclosed herein and a cytotoxic peptide moiety. Any cytotoxic peptide moiety known in the art can be used, including but not limited to ricin, Diphtheria toxin, and Pseudomonas exotoxin A. More exemplary cytotoxic peptides are disclosed in International Patent Application Publication Nos. WO2012/154530 and WO2014/164680. In certain embodiments, the cytotoxic peptide moiety is connected to the protein by chemical conjugation (e.g., covalent or noncovalent chemical conjugation). In certain embodiments, the cytotoxic peptide moiety is connected to the protein by fusion of polypeptide. The immunotoxin can further comprise an Fc domain connected to the antigen-binding site that binds CLEC12A. In certain embodiments, the immunotoxin comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, 82, 118, 119, 120, 121, 132, 133, 138, and 139. In certain embodiments, the cytotoxic peptide moiety is connected to the Fc domain directly or via a linker.

In another aspect, the present application provides a nucleic acid encoding at least one polypeptide of the immunotoxin, wherein the polypeptide comprises an antigen-binding site that binds CLEC12A. In certain embodiments, the nucleic acid further comprises a nucleotide sequence encoding a signal peptide that, when expressed, is at the N-terminus of one or more of the polypeptides of the immunotoxin. Also provided is a vector (e.g., a viral vector) comprising the nucleic acid, a producer cell comprising the nucleic acid or vector, and a producer cell expressing the immunotoxin.

### II. Therapeutic Compositions and Their Use

The present application provides methods for treating cancer using a protein, conjugate, or cells comprising an antigen-binding site disclosed herein and/or a pharmaceutical composition described herein. The methods may be used to treat a variety of cancers which express CLEC12A by administering to a patient in need thereof a therapeutically effective amount of a protein, conjugate, or cells comprising an antigen-binding site disclosed herein.

The therapeutic method can be characterized according to the cancer to be treated. For example, in certain embodiments, the cancer is acute myeloid leukemia, multiple myeloma, diffuse large B cell lymphoma, thymoma, adenoid cystic carcinoma, gastrointestinal cancer, renal cancer, breast cancer, glioblastoma, lung cancer, ovarian cancer, brain cancer, prostate cancer, pancreatic cancer, or melanoma. In some embodiments, the cancer is a hematologic malignancy or leukemia. In certain embodiments, the cancer is acute myeloid leukemia (AML), acute lymphoblastic leukemia (ALL), myelodysplasia, myelodysplastic syndromes, acute T-lymphoblastic leukemia, or acute promyelocytic leukemia, chronic myelomonocytic leukemia, or myeloid blast crisis of chronic myeloid leukemia.

In certain embodiments, the AML is a minimal residual disease (MRD). In certain embodiments, the MRD is characterized by the presence or absence of a mutation selected from CLEC12A-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)). In certain embodiments, the MDS is selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U). In certain embodiments, the MDS is a primary MDS or a secondary MDS.

In certain embodiments, the ALL is selected from B-cell acute lymphoblastic leukemia (B-ALL) and T-cell acute lymphoblastic leukemia (T-ALL). In certain embodiments, the MPN is selected from polycythaemia vera, essential thrombocythemia (ET), and myelofibrosis. In certain embodiments, the non-Hodgkin lymphoma is selected from B-cell lymphoma and T-cell lymphoma. In certain embodiments, the lymphoma is selected from chronic lymphocytic leukemia (CLL), lymphoblastic lymphoma (LPL), diffuse large B-cell lymphoma (DLBCL), Burkitt lymphoma (BL), primary mediastinal large B-cell lymphoma (PMBL), follicular lymphoma, mantle cell lymphoma, hairy cell leukemia, plasma cell myeloma (PCM) or multiple myeloma (MM), mature T/NK neoplasms, and histiocytic neoplasms.

In certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell can express one or more of the following in addition to CLEC12A: CD2, CD19, CD20, CD30, CD38, CD40, CD52, CD70, EGFR/ERBB1, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, and PD1.

In some embodiments, the cancer to be treated is selected from acute myeloid leukemia (AML), myelodysplastic syndrome (MDS), acute lymphoblastic leukemia (ALL), myeloproliferative neoplasms (MPNs), lymphoma, non-Hodgkin lymphomas, and classical Hodgkin lymphoma.

In some embodiments, the cancer to be treated is AML. In some embodiments of the present application, the AML is selected from undifferentiated acute myeloblastic leukemia, acute myeloblastic leukemia with minimal maturation, acute myeloblastic leukemia with maturation, acute promyelocytic leukemia (APL), acute myelomonocytic leukemia, acute myelomonocytic leukemia with eosinophilia, acute monocytic leukemia, acute erythroid leukemia, acute megakaryoblastic leukemia (AMKL), acute basophilic leukemia, acute panmyelosis with fibrosis, and blastic plasmacytoid dendritic cell neoplasm (BPDCN). In some embodiments, the present application provides treatment of AML characterized by expression of CLL-1 on the AML leukemia stem cells (LSCs). In some embodiments of the present application, the LSCs in an AML subject further express a membrane marker selected from CD34, CD38, CD123, TIM3, CD25, CD32, and CD96. In some embodiments of the present application, the AML is characterized as a minimal residual disease (MRD). In some embodiments of the present application, the MRD of AML is characterized by the presence or absence of a mutation selected from FLT3-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)).

In certain embodiments of the present application, the cancer is MDS selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U).

In certain embodiments of the present application, the ALL to be treated is selected from B-cell acute lymphoblastic leukemia (B-ALL) and T-cell acute lymphoblastic leukemia (T-ALL). In certain embodiments of the present application, the MPN to be treated is selected from polycythaemia vera, essential thrombocythemia (ET), and myelofibrosis. In certain embodiments of the present application, the non-Hodgkin lymphoma to be treated is selected from B-cell lymphoma and T-cell lymphoma. In certain embodiments of the present application, the lymphoma to be treated is selected from chronic lymphocytic leukemia (CLL), lymphoblastic lymphoma (LPL), diffuse large B-cell lymphoma (DLBCL), Burkitt lymphoma (BL), primary mediastinal large B-cell lymphoma (PMBL), follicular lymphoma, mantle cell lymphoma, hairy cell leukemia, plasma cell myeloma (PCM) or multiple myeloma (MM), mature T/NK neoplasms, and histiocytic neoplasms.

It is contemplated that the protein, conjugate, cells, and/or pharmaceutical compositions of the present disclosure can be used to treat a variety of cancers, not limited to cancers in which the cancer cells express CLEC12A. For example, in certain embodiments, the protein, conjugate, cells, and/or pharmaceutical compositions disclosed herein can be used to treat cancers that are associated with CLEC12A-expressing immune cells. CLEC12A is expressed on many myeloid lineages, and tumor-infiltrating myeloid cells (e.g., tumor-associated macrophages) may contribute to cancer progression and metastasis. Therefore, the methods disclosed herein may be used to treat a variety of cancers in which CLEC12A is expressed, whether on cancer cells or on immune cells.

### III. Combination Therapy

In another aspect, the present application provides for combination therapy. Proteins, conjugates, and cells comprising an antigen-binding site described herein can be used in combination with additional therapeutic agents to treat the cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins described in the present application can also be used as an adjunct to surgical removal of the primary lesion.

The amount of the protein, conjugate, or cells disclosed herein and the additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a protein, conjugate, or cell disclosed herein may be administered during a time when the additional therapeutic agent(s) exerts its prophylactic or therapeutic effect, or vice versa.

### IV. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In one aspect, the present disclosure provides a formulation of a protein, which contains a CLEC12A-binding site described herein, and a pharmaceutically acceptable carrier.

In certain embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:1, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:2. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:13, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:110, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:45, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:122, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:30. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:134, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:135. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:128, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:129. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:147, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:148. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:34. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:38. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:41, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:42. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:45, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:140. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:60, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:61. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:29, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:69. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:14, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:69. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:76, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:69. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:29, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:84. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:14, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:84. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:76, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:84. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:113, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:114. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:108, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:109. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:104, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:103. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:22, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:25. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:57, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:58. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:83, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:85. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:94, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:95.

The composition can be formulated for use in a variety of drug delivery systems. One or more physiologically acceptable excipients or carriers can be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

For example, this present disclosure could exist in an aqueous pharmaceutical formulation including a therapeutically effective amount of the protein in a buffered solution forming a formulation. Aqueous carriers can include sterile water for injection (SWFI), bacteriostatic water for injection (BWFI), a pH buffered solution (e.g. phosphate-buffered saline), sterile saline solution, Ringer's solution or dextrose solution. In certain embodiments, an aqueous formulation is prepared including the protein disclosed herein in a pH-buffered solution. The pH of the preparations typically will be between 3 and 11, more preferably between 5 and 9 or between 6 and 8, and most preferably between 7 and 8, such as 7 to 7.5. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g. 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/ml of disodium phosphate dihydrate, 0.7 to 1.1 mg/mL of sodium dihydrogen phosphate dihydrate, and 6.0 to 6.4 mg/mL of sodium chloride. The pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In some embodiments, the formulation includes an aqueous carrier, which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative, which is added to the formulations herein to reduce bacterial action. The addition of a preservative may, for example, facilitate the production of a multi-use (multiple-dose) formulation.

In some embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

Deamidation is a common product variant of peptides and proteins that may occur during fermentation, harvest/cell clarification, purification, drug substance/drug product storage and during sample analysis. Deamidation is the loss of NH3 from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation. In certain embodiments, the liquid formulation of the present disclosure may be preserved under conditions of pH and humidity to prevent deamination of the protein product.

In some embodiment, the formulation is a lyophilized formulation. In certain embodiments, the formulation is freeze-dried (lyophilized) and contained in about 12-60 vials. In certain embodiments, the formulation is freeze-dried and 45 mg of the freeze-dried formulation may be contained in one vial. In certain embodiments, the about 40 mg-about 100 mg of freeze-dried formulation is contained in one vial. In certain embodiments, freeze dried formulation from 12, 27, or 45 vials are combined to obtain a therapeutic dose of the protein in the intravenous drug formulation. The formulation may be a liquid formulation. In some embodiments, a liquid formulation is stored as about 250 mg/vial to about 1000 mg/vial. In certain embodiments, the liquid formulation is stored as about 600 mg/vial. In certain embodiments, the liquid formulation is stored as about 250 mg/vial.

In some embodiments, the lyophilized formulation includes the proteins described herein and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative. The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of a protein comprising an antigen-binding site described in the present application may contain such bulking agents.

In certain embodiments, the lyophilized protein product is constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution. In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution. In certain embodiments, the lyophilized protein product of the instant disclosure is constituted to about 4.5 mL water for injection and diluted with 0.9% saline solution (sodium chloride solution).

The protein compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The resulting compositions in solid form may be packaged in multiple single dose units, each containing a fixed amount of the above-mentioned agent or agents. The composition in solid form can also be packaged in a container for a flexible quantity.

Actual dosage levels of the active ingredients in the pharmaceutical compositions of of a protein comprising an antigen-binding site described in this application may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica. Chimica. Acta. 308: 43-53, 2001; Steimer et al., Clinica. Chimica. Acta. 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight. Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of a protein comprising an antigen-binding site described in the present application could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of a protein comprising an antigen-binding site described in the present application. The patent application specifically contemplates all combinations and permutations of the aspects and embodiments.

Throughout the description, where compositions are described as having, including, or comprising specific components, or where processes and methods are described as having, including, or comprising specific steps, it is contemplated that, additionally, there are compositions of a protein comprising an antigen-binding site described in the present application that consist essentially of, or consist of, the recited components, and that there are processes and methods according to the present application that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present application, whether explicit or implicit herein. For example, where reference is made to a particular compound, that compound can be used in various embodiments of compositions of a protein comprising an antigen-binding site described in the present application and/or in methods of a protein comprising an antigen-binding site described in the present application, unless otherwise understood from the context. In other words, within this application, embodiments have been described and depicted in a way that enables a clear and concise application to be written and drawn, but it is intended and will be appreciated that embodiments may be variously combined or separated without parting from the present teachings. For example, it will be appreciated that all features described and depicted herein can be applicable to all aspects of a protein comprising an antigen-binding site described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present application also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as a protein comprising an antigen-binding site described in the present application remain operable. Moreover, two or more steps or actions may be conducted simultaneously.

The use of any and all examples, or exemplary language herein, for example, “such as” or “including,” is intended merely to illustrate better a protein comprising an antigen-binding site described in the present application, and does not pose a limitation on the scope of a protein comprising an antigen-binding site described in the application unless claimed. No language in the specification should be construed as indicating any non-claimed element as essential to the practice of the present application.

### EXAMPLES

The following examples are merely illustrative and are not intended to limit the scope or content of the application in any way.

### Example 1. Characterization of Supernatants of Selected Hybridoma Clones

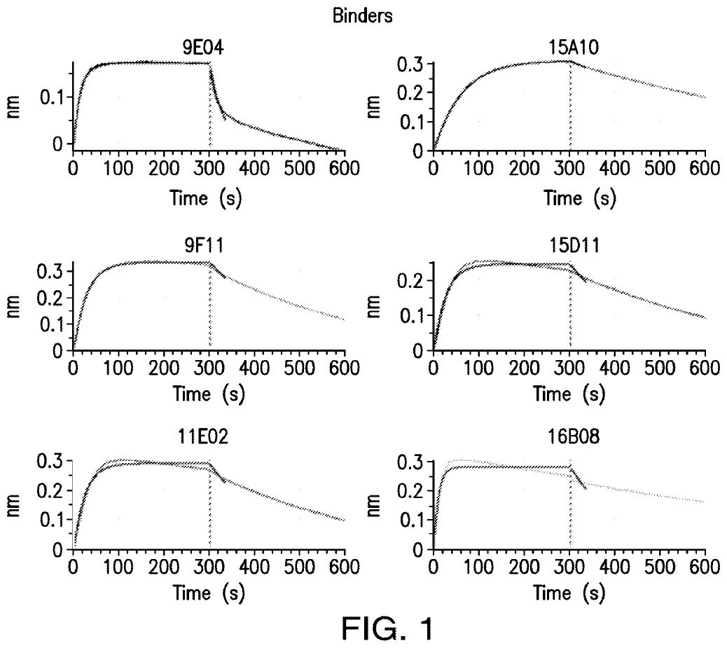

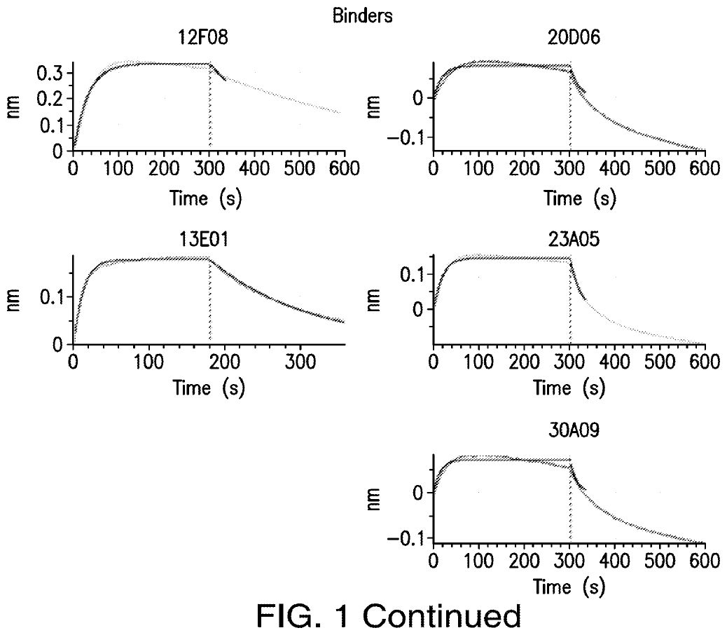

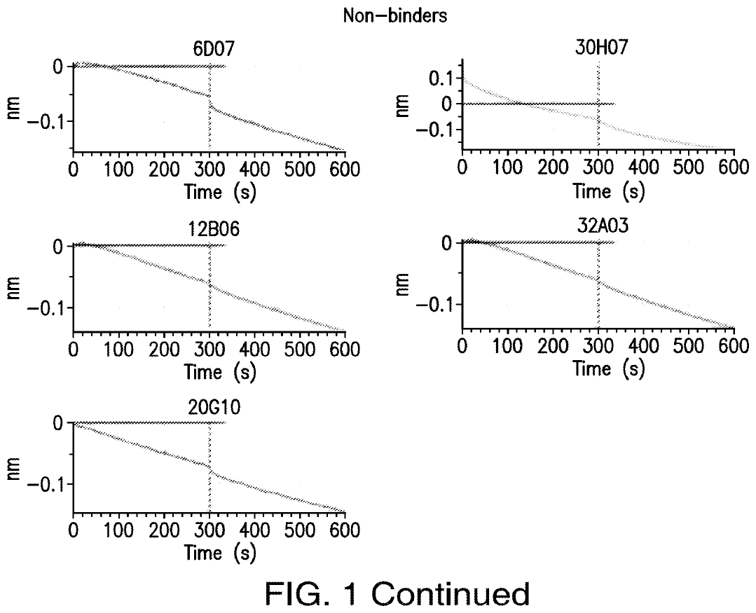

CLEC12A-specific antibodies were generated by immunizing BALB/c mice with hCLEC12A-His fusion protein. Supernatants of 16 hybridomas were assessed for CLEC12A binding by Bio-layer Interferometry (BLI) binding using an OctetRed384 (ForteBio). These 16 hybridomas were further analyzed for binding to human and cynomolgus CLEC12A expressed on the cell surface of isogenic cells; binding to cynomolgus CLEC12A was not observed. Estimated kinetic parameters are presented in Table 3 and binding traces are shown in FIG. 1. Nine clones were selected for further study. The ability of these nine clones to bind hCLEC12A-His RMA and CLEC12A-expressing cancer cell lines U937 and PL21 was further analyzed by high resolution surface plasmon resonance (SPR). The experiment was performed at 37° C. to mimic physiological temperature using a Biacore 8K instrument.

TABLE 3
Kinetic parameters and affinities of CLEC12A-His binding
to the antibodies produced from candidate hybridomas

| | | SPR at 37° C. | | | Cell Binding MFI | | |
|---|---|---|---|---|---|---|---|
| Test | Binning | kₐ | | K_{D} | RMA- | RMA- | |
| articles | profile | (1/Ms) | k_{d} (1/s) | (nM) | hCLEC12A | cCLEC12A | U937 |
| 9E04 | competitor | 247.0 | 3.51 × 10⁻² | 108.0 | 549.0 | 755.0 | 40.6 |
| 9F11 | competitor | 549.0 | 5.57 × 10⁻³ | 18.5 | 779.0 | 889.0 | 50.9 |
| 11E02 | — | 779.0 | 7.28 × 10⁻³ | 22.0 | 76.7 | 122.0 | 40.4 |
| 12F08 | unique | 76.7 | 6.23 × 10⁻³ | 23.7 | 79.5 | 97.4 | 46.0 |
| 13E01 | unique | 178.0 | 7.50 × 10⁻³ | 10.3 | — | — | — |
| 15A10 | unique | 79.5 | 2.23 × 10⁻³ | 15.8 | 372.0 | 167.0 | 83.8 |
| 15D11 | — | 372.0 | 6.64 × 10⁻³ | 20.7 | 71.1 | 90.5 | 44.0 |
| 16B08 | competitor | Heterogeneous binding | | | 819.0 | 1132.0 | 44.1 |
| 20D06 | unique | 819.0 | 5.25 × 10⁻² | 2310.0 | 90.2 | 179.0 | 46.5 |
| 23A05 | unique | 90.2 | 4.49 × 10⁻² | 446.0 | 77.2 | 92.9 | 42.9 |

| | | | | | |
|---|---|---|---|---|---|
| 30A09 | — | Heterogeneous binding | 74.7 | 149.0 | 42.8 |
| 6D07 | — | Non binder | 247.0 | 264.0 | 40.3 |
| 12B06 | — | Non binder | 178.0 | 335.0 | 41.5 |
| 20G10 | — | Non binder | 82.2 | 98.7 | 41.6 |
| 30H07 | — | Non binder | 76.3 | 121.0 | 45.7 |
| 32A03 | — | Non binder | 71.4 | 93.4 | 46.6 |

Binning of hybridoma fusions compared to reference mAbs was performed by BLI using OctetRed384 (ForteBio). Briefly, hybridoma supernatants were loaded onto anti-mouse IgG capture sensor tips for 15 minutes and equilibrated for 5 minutes in PBSF. Sensors were dipped into 200 nM hCLEC12A-His and allowed to associate for 180 seconds followed by dipping into 100 nM reference CLEC12A mAb. The increase in response units indicated that the hybridoma was a non-competitor to the reference mAb, whereas no increase in signal indicated that the hybridoma did compete with the reference mAb. The VH and VL sequences of these reference antibodies are provided in Table 4.

TABLE 4
Reference antibodies

| Anti- | | | |
|---|---|---|---|
| CLEC12A | | Sequence | |
| mAbs | Source | ID | Epitope |
| Merus- | Merus | VH | unknown |
| CLL1 | US | [SEQ ID NO: 123] | |
| | 2014/0120 | EVQLVQSGAEVKKPGAS | |
| | 096A1 | VKVSCKASGYTFTSYYM | |
| | | HWVRQAPGQGLEWMGII | |
| | | NPSGGSTSYAQKFQGRVT | |
| | | MTRDTSTSTVYMELSSLR | |
| | | SEDTAVYYCARGNYGDE | |
| | | FDYWGQGTLVTVSS | |
| | | VL | |
| | | [SEQ ID NO: 124] | |
| | | DIQMTQSPSSLSASVGDR | |
| | | VTITCRASQSISSYLNWY | |
| | | QQKPGKAPKLLIYAASSL | |
| | | QSGVPSRFSGSGSGTDFT | |
| | | LTISSLQPEDFATYYCQQ | |
| | | SYSTPPTFGQGTKVEIK | |
| Genentech- | Genentech | VH | C-type |
| h6E7 | US | [SEQ ID NO: 125] | lectin- |
| | 2016/0075 | DIQMTQSPSSLSASVGDR | like |
| | 787A1 | VTITCRASQSVSTSSYNY | domain, |
| | | MHWYQQKPGKPPKLLIK | residues |
| | | YASNLESGVPSRFSGSGS | 142-158 |
| | | GTDFTLTISSLQPEDFAT | |
| | | YYCQHSWEIPLTFGQGT | |
| | | KVEIK | |
| | | VL | |
| | | [SEQ ID NO: 126] | |
| | | EVQLVQSGAEVKKPGAS | |
| | | VKVSCKASGYSFTDYYM | |
| | | HWVRQAPGQGLEWIGRI | |
| | | NPYNGAAFYSQNFKDRV | |
| | | TLTVDTSTSTAYLELSSL | |
| | | RSEDTAVYYCAIERGADL | |
| | | EGYAMDYWGQGTLVTVS | |
| | | S | |

Binning analysis demonstrated that antibodies produced from five of the hybridomas, namely 12F8.G3, 13E01, 15A10.G8, 20D6.A8, and 23A5.D4, did not compete with the reference antibodies for binding to hCLEC12A-His. Binding of hybridomas to isogenic human CLEC12A (hCLEC12A) and cross-reactivity with cynomolgus monkey CLEC12A (cCLEC12A) were also evaluated by measuring the binding of the antibodies to isogenic RMA cells expressing CLEC12A and the U937 AML cancer cell line.

Briefly, RMA cells were transduced with a retroviral vector encoding cCLEC12A or hCLEC12A. Binding of the α-CLEC12A mAbs from crude hybridoma harvests to the hCLEC12A or cCLEC12A isogenic cell lines, as well as CLEC12A+U937 (ATCC catalog number CRL-1593.2) cancer cell lines, was performed as follows. 100,000 RMA or U937 cells were added per well of a 96 well round bottom plate. Cells were spun down and the pellet was gently dissociated by vortexing. 100 μL of Zombie live/dead dye (PBS+1:2000 dye) were added per well and incubated in the dark at room temperature for 20 minutes. Cells were washed with 200 μL of FACS buffer (PBS+2% FBS). 50 μL of hybridoma supernatants were added to the washed cells and the mixtures were incubated for 30 minutes on ice in the dark. Cells were washed twice in FACS buffer, 50 μL of anti-mouse Fc-PE secondary reagent (1:200 dilution) were added and the mixture was incubated for 20 minutes on ice in the dark. After the incubation, the cells were washed in FACS buffer and then fixed with 50 μL of 4% paraformaldehyde for 15 minutes on ice. The fixed cells were washed, and then resuspended in 200 μL FACS buffer and stored at 4° C. until ready for acquisition. The samples of cells resuspended in FACS buffer were run on BD FACSCelesta equipped with an HTS (high throughput sampler) to determine the binding affinities of the antibodies to isogenic RMA cells expressing CLEC12A and the U937 AML cancer cell line.

The binding affinities of the hybridoma supernatants to PL21 AML cancer cells (DSMZ catalog number ACC536), a human AML cell line reported to express CLEC12A, were also measured. As shown in Table 3 supra, nine of the clones displayed binding affinity to cancer cells expressing hCLEC12A. Binding to cCLEC12A was not observed.

### Example 2. Analysis of Purified Anti-CLEC12A Murine Antibodies

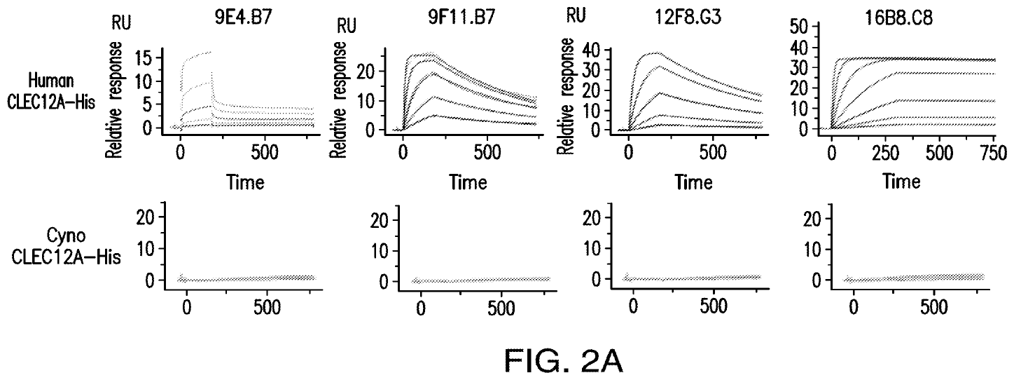

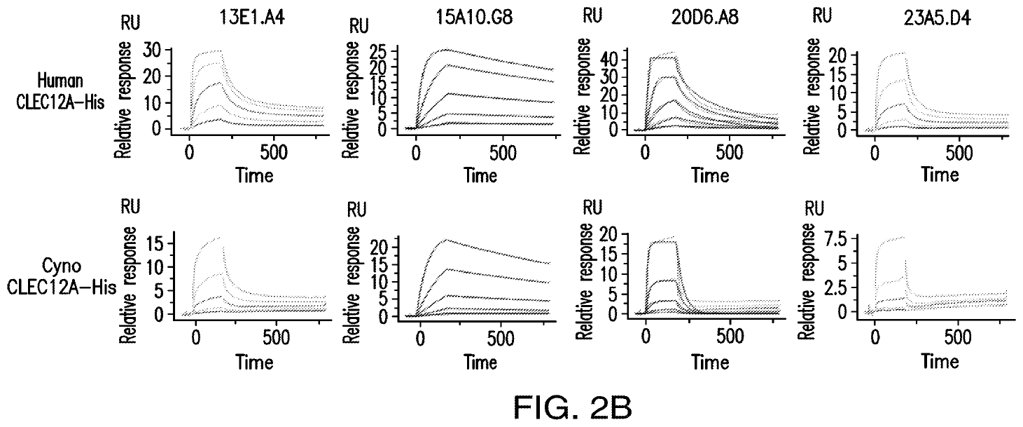

In this Example, kinetic parameters and binding affinities of the purified anti-CLEC12A murine antibodies were analyzed. Based on the analysis described in Example 1, eight hybridomas (9F11.B7, 12F8.G3, 16B8.C8, 15A10.G8, 20D6.A8, 9E4.B7, 13E1.A4, and 23A5.D4) were selected for subcloning and sequencing. Each subclone was purified from the hybridoma culture, and binding to hCLEC12A-His and cCLEC12A-His was assessed by SPR. The data from these experiments are shown in FIG. 2. Antibodies 9E4.B7, 9F11.B7, 12F8.G3, and 16B8.C8 bound to hCLEC12A only (FIG. 2A); whereas antibodies 13E1.A4, 15A10.G8, 20D6.A8, and 23A5.D4 bound to both hCLEC12A and cCLEC12A (FIG. 2B). Kinetic constants and binding affinities of hCLEC12A and cCLEC12A to purified murine subcloned mAbs are provided in Table 5.

TABLE 5
Kinetic parameters and affinities of hCLEC12A
binding to purified murine subclones

| | Human CLEC12A-His | | | Cyno CLEC12A-His | | |
|---|---|---|---|---|---|---|
| Test | kₐ | | K_{D} | kₐ | | K_{D} |
| Article | (M⁻¹s⁻¹) | k_{d}(s⁻¹) | (nM) | (M⁻¹s⁻¹) | k_{d}(s⁻¹) | (nM) |
| 9E4.B7 | Low affinity heterogenous interaction | | | No binding | | |
| 9F11.B7 | 1.14 × 10⁶ | 1.57 × 10⁻³ | 1.4 | No binding | | |
| 12F8.G3 | 3.97 × 10⁵ | 1.39 × 10⁻³ | 3.5 | No binding | | |
| 16B8.C8* | 4.05 × 10⁶ | 4.44 × 10⁻⁵ | 0.01 | No binding | | |
| 13E1.A4 | Low affinity heterogenous interaction | | | Low affinity heterogenous interaction | | |
| 15A10.G8 | 3.01 × 10⁵ | 4.94 × 10⁻⁴ | 1.6 | 1.96 × 10⁵ | 6.59 × 10⁻⁴ | 3.3 |
| 20D6.A8 | 1.31 × 10⁷ | 9.50 × 10⁻² | 7.2 | 9.16 × 10⁵ | 2.46 × 10⁻² | 26 |

| | | |
|---|---|---|
| 23A5.D4 | Low affinity heterogenous interaction | Low affinity heterogenous interaction |

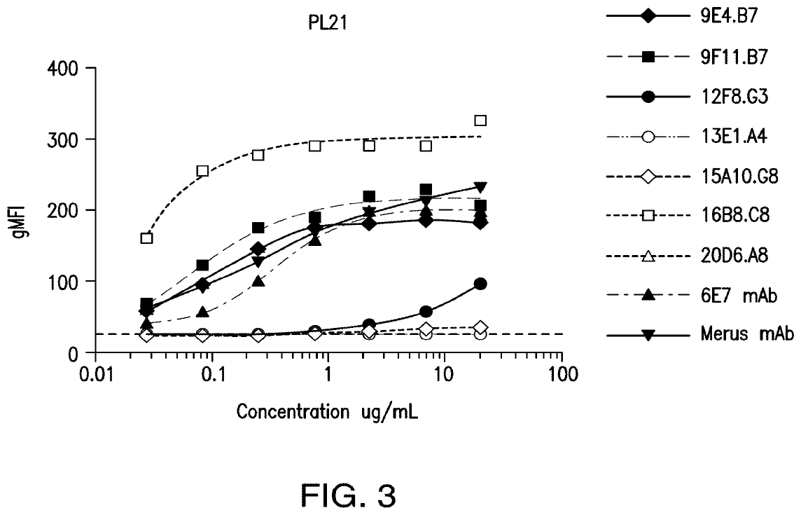

The ability of the eight purified subcloned mAbs to bind to CLEC12A expressing cells was assessed by FACS analysis with human CLEC12A+PL21 cancer cell line. As shown in FIG. 3, 9E4.B7, 9F11.B7 and 16B8.C8 all bound to the PL21 cell line with sub-nanomolar EC50 values; however, only 9F11.B7 and 16B8.C8 satisfied both recombinant protein binding and cell binding criteria, as 9E4.B7 demonstrated low affinity heterogenous binding to recombinant hCLEC12A-His (Table 6). Although 15A10.G8, 13E1.A4, 20D6.A8 and 23A5.D4 showed binding to recombinant human and cyno CLEC12A in the SPR assay, these mAbs failed to recognize cancer cells, suggesting conformational differences in the binding epitope between recombinant and cell surface expressed CLEC12A. As demonstrated, both Merus-CLL1 and Genentech-h6E7 mAbs bound to PL21 with significantly inferior EC50 values as compared to the novel CLEC12A hybridoma clones.

TABLE 6
Cell binding confirmation of purified
mouse mAbs to human PL21 cell line

| Test article | EC₅₀ (nM) | Max MFI |
|---|---|---|
| 9E4.B7 | 0.64 | 186 |
| 9F11.B7 | 0.56 | 217 |
| 12F8.G3 | Non binder | n/a* |
| 16B8.C8 | 0.18 | 300 |
| 13E1.A4 | Non binder | n/a |
| 15A10.G8 | Non binder | n/a |
| 20D6.A8 | Non binder | n/a |
| 23A5.D4 | Non binder | n/a |
| Merus-CLL1^{#} | 2.02 | 201 |
| Genentech-h6E7^{#} | 5 | 265 |

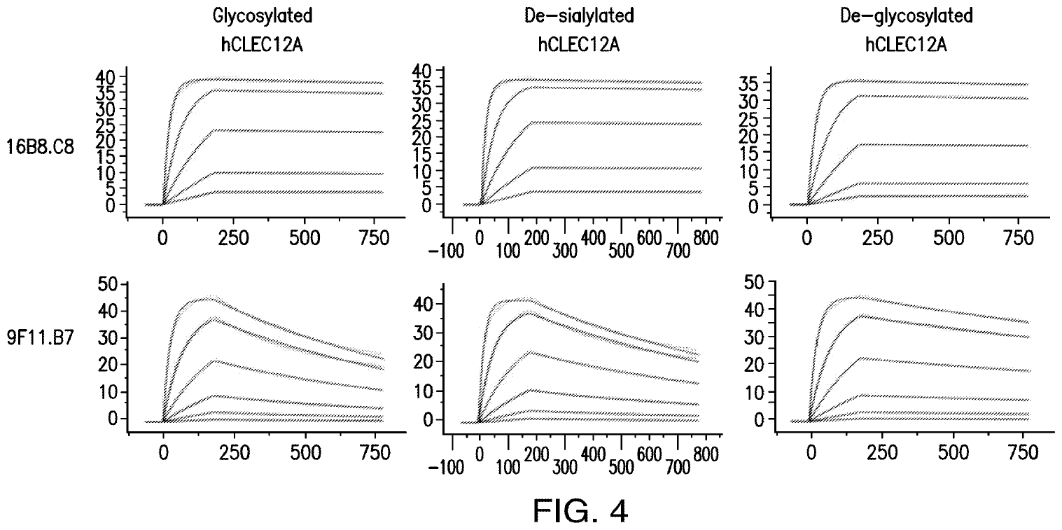

To assess if mAbs bind to CLEC12A in glycosylation independent manner, binding of clones 16B8.C8 and 9F11.B7 to glycosylated, de-sialylated and PNGase-treated hCLEC12A was assessed by SPR. As demonstrated by the sensorgrams in FIG. 4 and the quantification in Table 7, both 16B8.C8 and 9F11.B7 bound to de-sialylated and deglycosylated versions of hCLEC12A without loss of affinity, suggesting that the antibody interactions with hCLEC12A are not affected by glycosylation status of the target.

TABLE 7
Kinetic parameters and affinities of 16B8.C8 and 9F11.B7
to differentially glycosylated hCLEC12A by SPR.

| | | kₐ | k_{d} | K_{D} |
|---|---|---|---|---|
| Test article | Analyte | (M⁻¹s⁻¹) | (s⁻¹) | (nM) |
| Murine 16B8. mAb | Glycosylated hCLEC12A | 4.05 × 10⁶ | 4.70 × 10⁻⁵ | 0.012 |
| Murine 16B8 mAb | De-sialylated hCLEC12A | 5.25 × 10⁶ | 3.62 × 10⁻⁵ | 0.007 |
| Murine 16B8 mAb | De-glycosylated hCLEC12A | 4.26 × 10⁶ | 4.45 × 10⁻⁵ | 0.011 |
| Murine F3′-9F11 | Glycosylated hCLEC12A | 1.15 × 10⁶ | 1.11 × 10⁻³ | 0.96 |
| Murine F3′-9F11 | De-sialylated hCLEC12A | 1.58 × 10⁶ | 9.80 × 10⁻⁴ | 0.62 |
| Murine F3′-9F11 | De-glycosylated hCLEC12A | 1.11 × 10⁶ | 3.74 × 10⁻⁴ | 0.34 |

### Example 3. Putative Sequence Liability Analysis

Potential sequence liabilities in CDRs (identified under Chothia) of the 16B8.C8 and 9F11.B7 antibodies were examined. The following potential liabilities were considered: M (potential oxidation site); NG, NS and NT sequence motif (potential deamidation site); DG, DS and DT sequence motif (potential isomerization site); DP sequence motif (potential site for chemical hydrolysis). The results are summarized in Table 8.

TABLE 8
Putative sequence liabilities in the CDRs of selected murine mAbs

| | Potential sequence | |
|---|---|---|
| Clone ID | liability motif | Location |
| 16B8.C8 | DS (Isomerization site) | CDRH3 |
| 9F11.B7 | M (Oxidation site) | CDRH3 |
| | DG (Isomerization site) | CDRH3 |
| | NS (Deamidation site) | CDRH1 |

Variants of these antibodies were designed to remove the putative sequence liability motifs.

### Example 4. Humanization of Subclones

Based on the data collected regarding kinetics and affinity for recombinant hCLEC12A protein, binding to cell surface expressed hCLEC12A, and binding to AML cancer cell lines, two mouse hybridoma subclones, namely 16B8.C8 and 9F11.B7, were selected for humanization.

The 16B8.C8 antibody was humanized. Back mutations were introduced in the framework regions to create variants having the VH and VL sequences of scFv-1292 to scFv-1309, and scFv-1602 and scFv-2061.

The 9F11.B7 antibody was humanized. Back mutations were introduced in the framework regions to create variants AB0186 to AB0196.

### Example 5. Humanized CLEC12A Binder Analysis

Binding affinities of the 18 humanized scFv variants of clone 16B8.C8 for hCLEC12A, and multispecific binding proteins derived from said variants, were assessed by SPR in screening. The multispecific binding proteins comprise an scFv that binds CLEC12A, at least one antigen-binding site that binds an unrelated protein, and an antibody Fc region. A multispecific binding protein as described in the Examples infra is referred to herein by hF3′ or F3′, followed by the CLEC12A scFv variant it comprises, e.g., hF3′-1602 comprises scFv-1602.

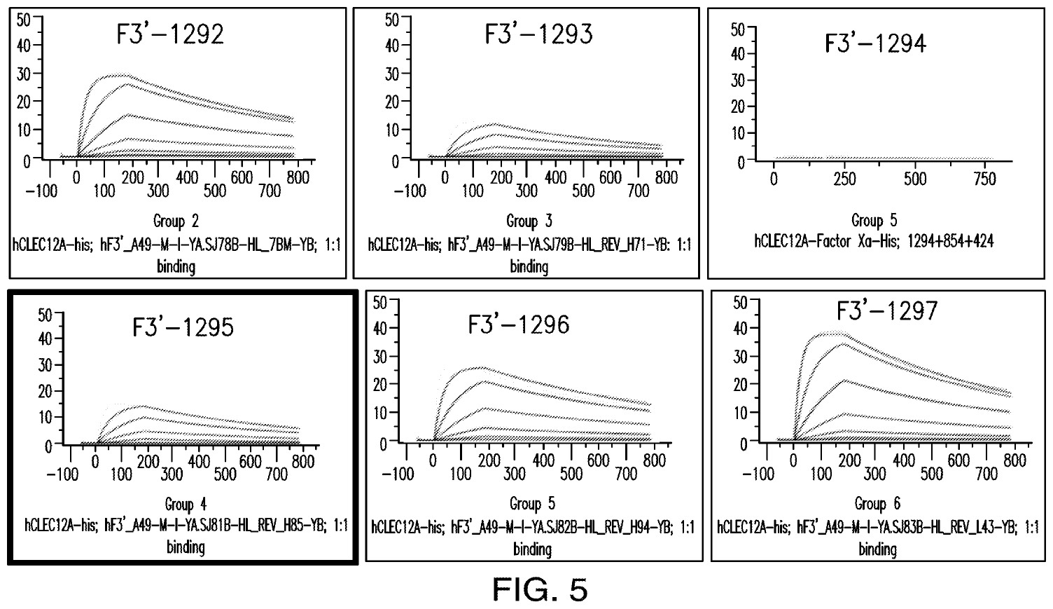

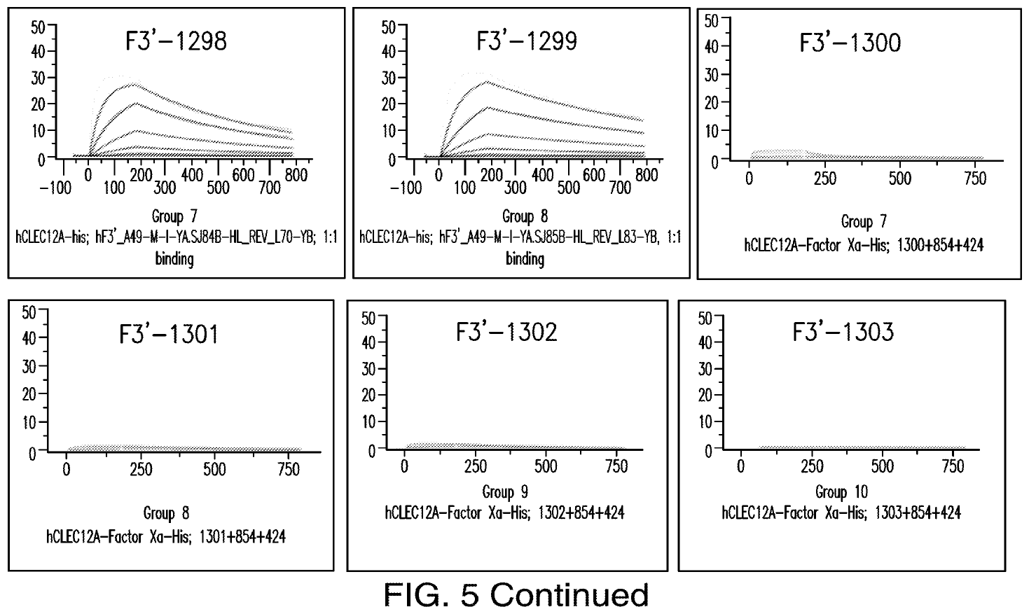

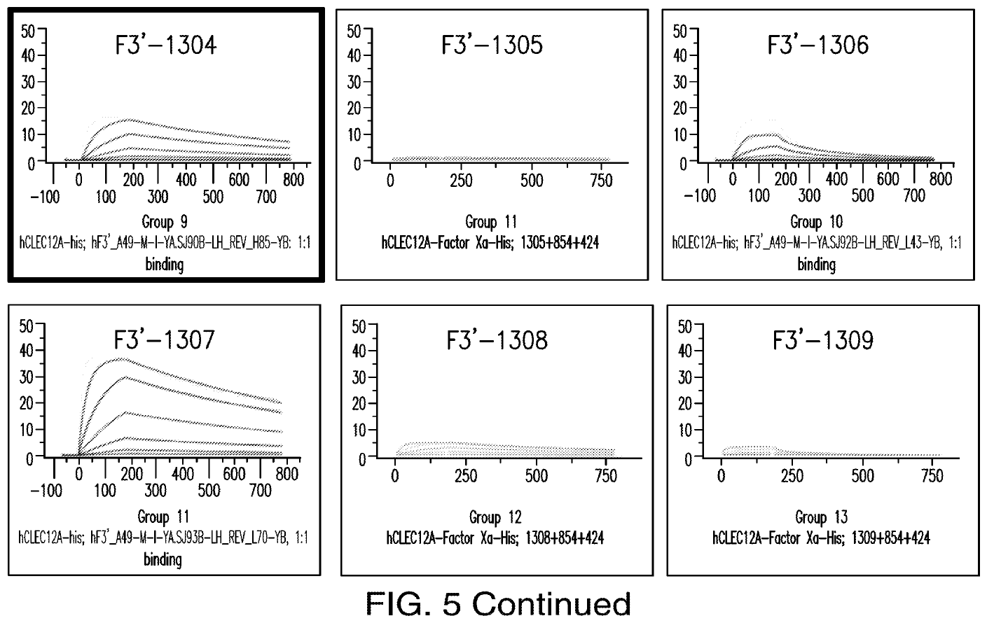

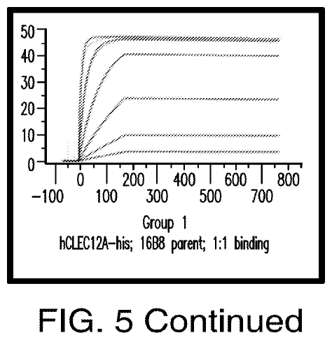

Binding signals for eight multispecific binding proteins were less than 5 RU (approximately 15% of the expected signal ran in this assay) and, therefore, these multispecific binding proteins were considered as nonbinders to hCLEC12A. The remaining ten multispecific binding proteins bound to hCLEC12A with <10 nM affinity, as shown in FIG. 5 and Table 9. However, a potential N-glycosylation site was involuntarily introduced in eight out of the ten multispecific binding proteins by introducing murine back mutation N in position H85. Only constructs F3′-1295 and F3′-1304 (containing A in position H85) did not present the N-glycosylation sequence liability; therefore, only these two constructs were carried forward for further characterization.

TABLE 9
Kinetics and affinities of hCLEC12A binding to semi-
purified humanized multispecific binding proteins.

| | | Number of back | | | | |
|---|---|---|---|---|---|---|
| | | mutations and | N-glycosylation | | | |
| Test | scFv | residues reverted | sequence | kₐ | | K^{D} |
| article | orientations | to human | liability site | (M⁻¹s⁻¹) | kₐ (s⁻¹) | (nM) |
| F3′-1292 | VH-VL | 7 BM | Yes | 4.02 × 10⁵ | 1.30 × 10⁻³ | 3.22 |
| F3′-1293 | VH-VL | 6 BM, K(H71)V* | Yes | 4.58 × 10⁵ | 1.75 × 10⁻³ | 3.81 |
| F3′-1294 | VH-VL | 6 BM, Q(H83)T | Yes | Binding signal < 5RU | | |
| F3′-1295 | VH-VL | 6 BM, N(H85)A | No | 5.20 × 10⁵ | 1.53 × 10⁻³ | 2.94 |
| F3′-1296 | VH-VL | 6 BM, K(H94)R | Yes | 8.74 × 10⁵ | 1.17 × 10⁻³ | 1.34 |
| F3′-1297 | VH-VL | 6 BM, I(L43)A | Yes | 5.04 × 10⁵ | 1.41 × 10⁻³ | 2.79 |
| F3′-1298 | VH-VL | 6 BM, R(L70)D | Yes | 6.44 × 10⁵ | 1.85 × 10⁻³ | 2.87 |
| F3′-1299 | VH-VL | 6 BM, I(L83)F | Yes | 4.26 × 10⁵ | 1.21 × 10⁻³ | 2.83 |
| F3′-1300 | VH-VL | No BM | No | Binding signal < 5RU | | |
| F3′-1301 | VL-VH | 7 BM | Yes | Binding signal < 5RU | | |
| F3′-1302 | VL-VH | 6 BM, K(H71)V | Yes | Binding signal < 5RU | | |
| F3′-1303 | VL-VH | 6 BM, Q(H83)T | Yes | Binding signal < 5RU | | |
| F3′-1304 | VL-VH | 6 BM, N(H85)A | No | 4.80 × 10⁵ | 1.30 × 10⁻³ | 2.70 |
| F3′-1305 | VL-VH | 6 BM, K(H94)R | Yes | Binding signal < 5RU | | |
| F3′-1306 | VL-VH | 6 BM, I(L43)A | Yes | 9.48 × 10⁶ | 4.89 × 10⁻² | 5.16 |
| F3′-1307 | VL-VH | 6 BM, R(L70)D | Yes | 9.14 × 10⁵ | 1.00 × 10⁻³ | 1.10 |
| F3′-1308 | VL-VH | 6 BM, I(L83)F | Yes | Binding signal < 5RU | | |
| F3′-1309 | VL-VH | No BM | No | Binding signal < 5RU | | |
| m16B8.C8** | Mouse mAb | | Yes | 3.03 × 10⁶ | 2.35 × 10⁻⁵ | 0.008 |

*In the “residues reverted to human,” the first letter is a murine residue, and the letter and number in the parentheses indicate positions of reverted residues in the heavy chain and light chain, and the last letter is the human residue, e.g., in K(H71)V, murine K in position 71 of the heavy chain is replaced by human residue V.
**murine parental mAb 16B8.C8 was fully purified.

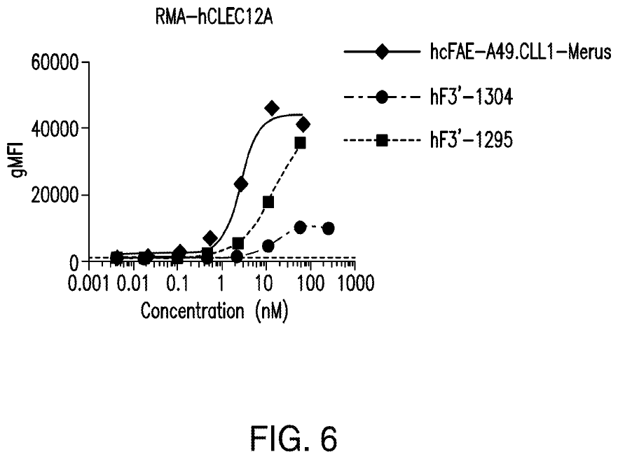

F3′-1295 and F3′-1304 were tested for binding isogenic to hCLEC12A+RMA cells, as shown in FIG. 6. Binding of F3′-1295 to cell-surface expressed hCLEC12A was comparable to hcFAE-A49.CLL1-Merus control multispecific binding protein, which is derived from the Merus antibody described supra, whereas binding of F3′-1304 to cell-surface expressed hCLEC12A was poorer compared to the hcFAE-A49.CLL1-Merus control multispecific binding protein.

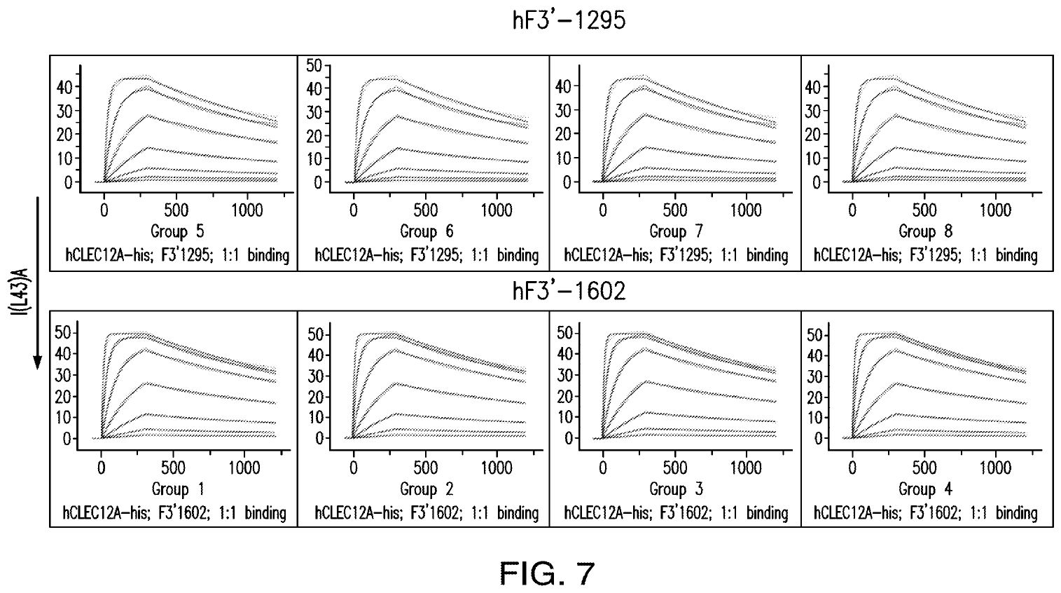

To determine if the human residue in position L43 was responsible for an increase in thermostability, in both F3′-1306 and F3′-1297, the mouse residue I(L43) was reverted back to the original human framework residue A(L43). Moreover, the murine Ile in position L43 of F3′-1295 was replaced with human Ala, thereby generating F3′-1602. To understand if the I(L43)A substitution had an effect on the multispecific binding protein affinity for hCLEC12A, binding of hCLEC12A to F3′-1295 and F3′-1602 was determined by SPR (Biacore) at 37° C. (FIG. 7), and using the methods as described in Example 1 supra. The kinetic constants and equilibrium binding affinities are listed in Table 10. Both multispecific binding proteins have very similar off rates, but F3′-1602 displays about 2-fold lower K_{D} due to its faster on rate. Therefore, the I(L43)A substitution had an effect on the K_{D}.

TABLE 10
Kinetic parameters and affinities of F3′-1295
and F3′-1602 binding to hCLEC12A by SPR.

| | | kₐ | k_{d} | K_{D} |
|---|---|---|---|---|
| Test article | Target | (M⁻¹s⁻¹) | (s⁻¹) | (nM) |
| F3′1295 | hCLEC12A-His | 4.11 × 10⁵ | 5.87 × 10⁻⁴ | 1.43 |
| | hCLEC12A-His | 4.15 × 10⁵ | 5.89 × 10⁻⁴ | 1.42 |
| | hCLEC12A-His | 4.19 × 10⁵ | 6.05 × 10⁻⁴ | 1.44 |
| | hCLEC12A-His | 4.16 × 10⁵ | 5.96 × 10⁻⁴ | 1.43 |
| Average ± StDev | | (4.15 ± 0.03) × 10⁵ | (5.94 ± 0.09) × 10⁻⁴ | 1.43 ± 0.01 |
| F3′1602 | hCLEC12A-His | 8.54 × 10⁵ | 4.94 × 10⁻⁴ | 0.58 |
| | hCLEC12A-His | 8.33 × 10⁵ | 4.94 × 10⁻⁴ | 0.59 |
| | hCLEC12A-His | 8.63 × 10⁵ | 4.89 × 10⁻⁴ | 0.57 |
| | hCLEC12A-His | 8.26 × 10⁵ | 4.99 × 10⁻⁴ | 0.60 |

| | | | |
|---|---|---|---|
| Average ± StDev | (8.44 ± 0.17) × 10⁵ | (4.94 ± 0.04) × 10⁻⁴ | 0.57 ± 0.01 |

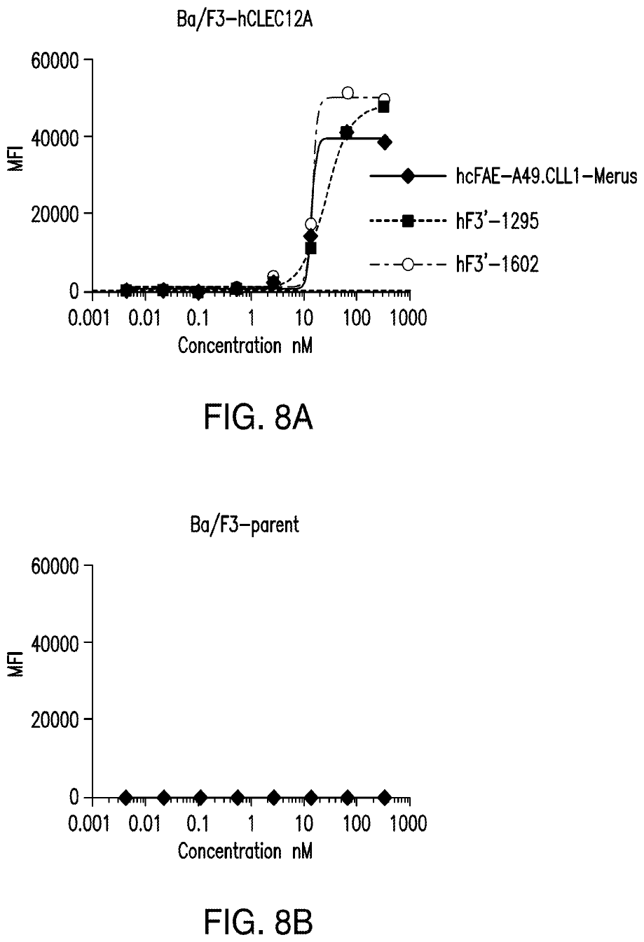

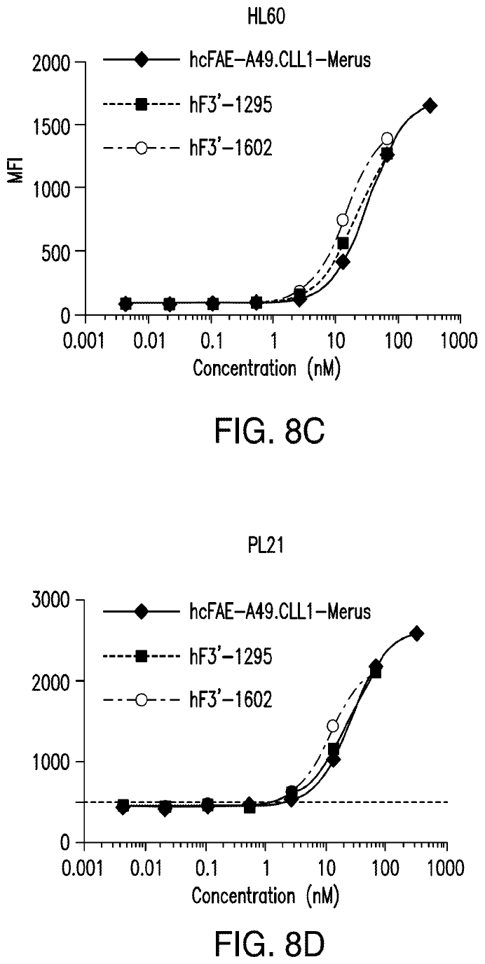

F3′-1295 and F3′-1602 were tested for their ability to bind isogenic Ba/F3 cells expressing human CLEC12A, in comparison to the parental Ba/F3 cells (shown in FIG. 8A and the data are listed in Table 11). F3′-1602 was able to bind hCLEC12A+Ba/F3 with about 2-fold lower EC50 value but with similar maximum binding MFI compared to F3′-1295. No binding to the parental Ba/F3 cell lacking expression of CLEC12A was detected by either F3′-1295 or F3′-1602 (FIG. 8B), suggesting high specificity binding of multispecific binding protein to hCLEC12A.

TABLE 11
EC50 and max MFI of multispecific binding protein
binding to hCLEC12A + Ba/F3 isogenic cell line.

| | hCLEC12A + Ba/ | | Ba/F3 parental | |
|---|---|---|---|---|
| | F3 cell line | | cell line | |
| | EC50 | Max | EC50 | Max |
| Test article | (nM) | MFI | (nM) | MFI |
| Z | Z | Z | Z | Z |

| | | | |
|---|---|---|---|
| F3′-1295 | 26.1 | 48040 | No binding |
| F3′-1602 | 14.4 | 50160 | No binding |
| hcFAE-A49.CLL-Merus | 14.3 | 39740 | No binding |

F3′-1295 and F3′-1602 were further tested for their ability to bind HL60 (FIG. 8C) and PL21 (FIG. 8D) AML cancer cell lines. F3′-1602 was able to bind to both cell lines with lower EC50 values but with similar maximum binding MFI as compared to F3′-1295.

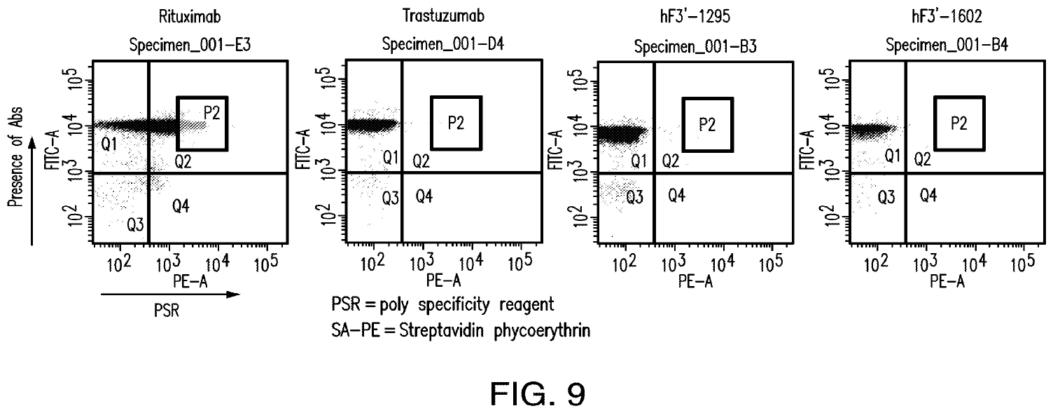

Off-target effects of a drug need to be evaluated when developing protein therapeutics. A flow cytometry based polyspecificity reagent (PSR) assay allows for determination of antibodies that have a higher probability to bind non-specifically. F3′-1295 and F3′-1602 were tested for non-specific binding to a preparation of detergent solubilized CHO cell membrane proteins in the PSR assay. Both humanized F3′-1602 and F3′-1295 did not bind to PSR (no signal shift to the right) and showed very similar profiles as the PSR control Trastuzumab, demonstrated in FIG. 9, suggesting high specificity of the multispecific binding proteins. Rituximab was used as positive control in this assay. F3′-1602 was selected for further comparison with additional multispecific binding proteins described in the following experiments.

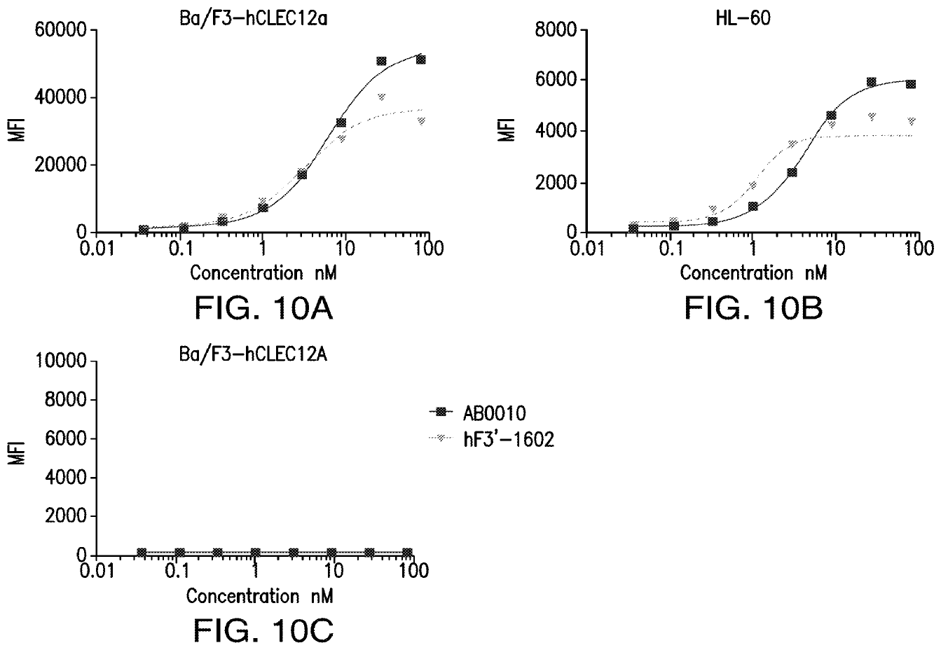

The effect of different multispecific binding protein format was tested experimentally for alterations in efficacy. Multispecific binding proteins of different formats, F3′-1602 and AB0010, were tested for their abilities to bind Ba/F3 cells expressing human CLEC12A (FIG. 10A) and AML cancer cell line (FIG. 10B). AB0010 comprises scFv-1602 in Fab format rather than scFv; the unrelated protein binder, by contrast, is present as an scFv. The EC50 value for F3′-1602 was superior over AB0010 in HL-60 cells, about 2-fold decreased (Table 12). Neither AB0010 nor F3′-1602 showed any binding to the parental Ba/F3 cells, demonstrating high specificity of binding to CLEC12A (FIG. 10C).

TABLE 12
Binding EC50 and max MFI of F3′-1602 and AB0010
to hCLEC12A + Ba/F3 and HL60 AML cells.

| | | hCLEC12A + | | | | Ba/F3 parent | |
|---|---|---|---|---|---|---|---|
| | | Ba/F3 cell line | | HL60 | | cell line | |
| | Test | Max | EC50 | Max | EC50 | Max | EC50 |
| Format | article | MFI | (nM) | MFI | (nM) | MFI | (nM) |

| | | | | | | |
|---|---|---|---|---|---|---|
| F3′ | F3′-1602 | 36920 | 3.20 | 3820 | 1.1 | No binding |
| F3 | AB0010 | 54827 | 6.13 | 6064 | 4.2 | No binding |

For generation of 9F11.B7 multispecific binding proteins, all 12 scFv variants of 9F11.B7 were combined with an unrelated protein binder and an antibody Fc domain. Affinities of the 10 semi-purified (Protein A) multispecific binding proteins for hCLEC12A were assessed by SPR in the screening mode shown in Table 13. Three (AB0190, AB0193 and AB0196) 9F11.B7 based multispecific binding proteins showed heterogenous binding and could not be fitted to a 1:1 kinetic model with high confidence. The binding kinetics of the remaining 7 multispecific binding proteins were similar to the chimeric parent mouse 9F11.B7 mAb, suggesting that neither humanization nor conversion of Fab to scFv affected the affinity for hCLEC12A.

TABLE 13
Kinetics and affinities of hCLEC12A binding to semi-
purified, humanized muti-specific binding protein.

| | kₐ | k_{d} | K_{D} | |
|---|---|---|---|---|
| Test article | (M⁻¹s⁻¹) | (s⁻¹) | (nM) | Comments |
| AB0185 | 7.26 × 10⁵ | 1.01 × 10⁻³ | 1.40 | |
| AB0186 | 1.12 × 10⁶ | 1.17 × 10⁻³ | 1.04 | |
| AB0188 | 5.69 × 10⁵ | 9.01 × 10⁻⁴ | 1.58 | Low heterodimer yield |
| AB0189 | 1.04 × 10⁶ | 1.27 × 10⁻³ | 1.21 | |
| AB0190 | 1.55 × 10⁷ | 1.77 × 10⁻² | 1.14 | Estimated/heterogeneous binding |
| AB0191 | 7.28 × 10⁵ | 1.26 × 10⁻³ | 1.73 | |
| AB0192 | 1.09 × 10⁶ | 1.38 × 10⁻³ | 1.26 | |
| AB0193 | 1.97 × 10⁷ | 2.07 × 10⁻² | 1.04 | Estimated/heterogeneous binding |
| AB0195 | 7.66 × 10⁵ | 1.29 × 10⁻³ | 1.68 | |
| AB0196 | Heterogenous weak binding; data insufficient for estimation | | | |
| m9F11-hIgG1 | 9.50 × 10⁵ | 1.02 × 10⁻³ | 1.07 | |

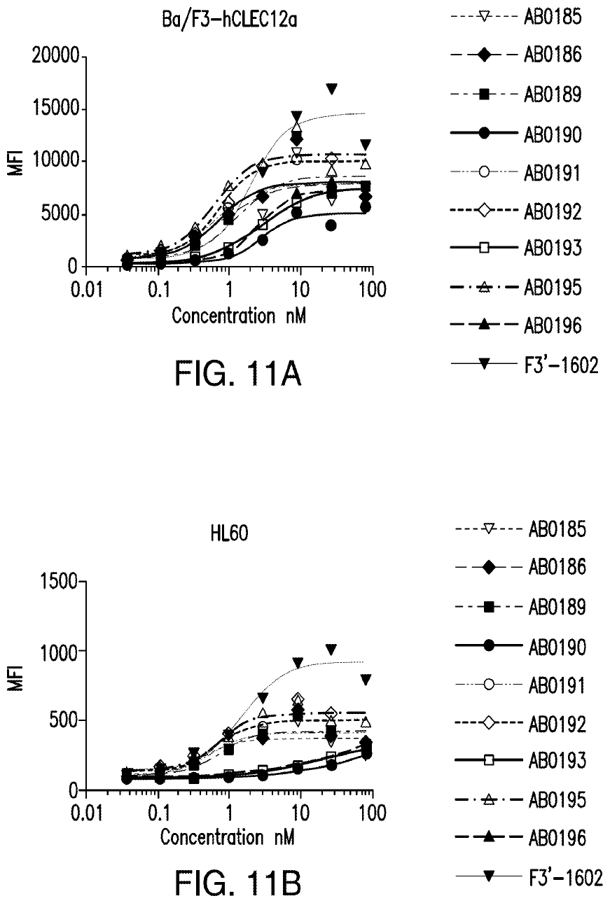

Binding of 9 fully purified humanized 9F11.B7 multispecific binding proteins to hCLEC12A+Ba/F3 and HL60 AML cancer cell lines, shown in FIG. 11A and FIG. 11B, was tested by FACS. AB0190, AB0193 and AB0196 showed inferior binding EC50 values for both cell lines (Table 14), which correlates with poor behavior in SPR described in Table 12. EC50 values for the remaining 6 clones were similar, showing correlation with the SPR data.

TABLE 14
Binding EC50 and max MFI of 9F11.B7 based multispecific binding
proteins to hCLEC12A + Ba/F3 isogenic and HL60 AML cell lines

| | |
|---|---|
| hCLEC12A + Ba/ | HL60 cancer |
| F3 isogenic cell line | cell line |

| | EC50 | Max binding | EC50 | Max binding |
|---|---|---|---|---|
| Test Article | (nM) | MFI | (nM) | MFI |
| AB0185 | 0.7 | 8025 | 0.4 | 372 |
| AB0186 | 0.7 | 8160 | 0.5 | 412 |
| AB0189 | 1.3 | 8760 | 0.7 | 420 |
| AB0190 | 2.9 | 5139 | N/A | n/a |
| AB0191 | 0.6 | 7941 | 0.6 | 420 |
| AB0192 | 0.7 | 10158 | 0.6 | 503 |
| AB0193 | 2.9 | 7732 | 19 | 345 |
| AB0195 | 0.6 | 10775 | 0.7 | 551 |
| AB0196 | 2.7 | 7427 | 135 | 650 |
| F3′-1602 | 2.0 | 14712 | 1.6 | 924 |

### Example 6. Molecular Format and Design, Structure, Affinity, Potency, Specificity and Cross-Reactivity Analysis of F3′-1602

### Surface Plasmon Resonance (SPR)

Binding affinities of F3′-1602 for recombinant human CLEC12A (hCLEC12A) or cyno CLEC12A (cCLEC12A) were measured by SPR using a Biacore 8K instrument at physiological temperature of 37° C. Briefly, human Fc specific antibodies were covalently immobilized at a density of about 8000-10000 resonance units (RU) on carboxy methyl dextran matrix of a CM5 biosensor chip to create an anti-hFc IgG chip. F3′-1602 samples were injected on the anti-hFc IgG chip at a flow rate of 10 μL/min for 60 seconds to achieve an about 250 RU capture level. hCLEC12A-His or cCLEC12A-His was serially diluted (100 nM-0.14 nM) in three-fold dilutions with running buffer and injected at a flow rate of 30 μl/min over the captured test articles. Association was monitored for 300 seconds, and dissociation was monitored for 900 seconds. Surfaces were regenerated between cycles with three pulses of 10 mM glycine-HCl (pH 1.7) injected for 20 seconds at 100 μL/min.

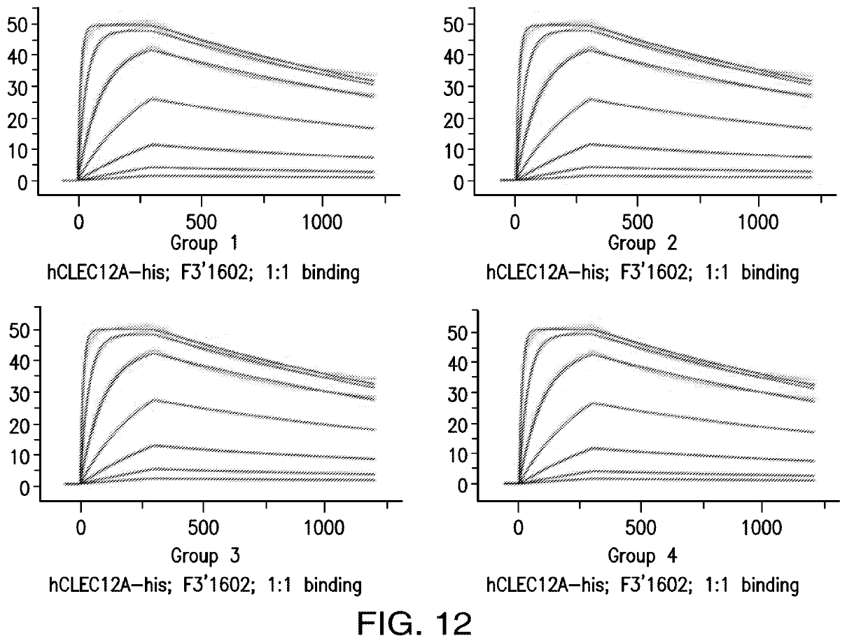

The kinetic constants and equilibrium binding affinities are provided in Table 15, and raw data and fits are shown in FIG. 12. The complex between F3′-1602 and human CLEC12A is strong as evidenced from the slow rate dissociation constant 4.94±0.09×10⁻⁴ s⁻¹. The equilibrium binding affinity K_{D} for F3′-1602 was 0.59±0.01 nM.

TABLE 15
Comparison of kinetic parameters of F3′-1602 to human CLEC12A at pH 7.4 and pH 6.0.

| | | kₐ | k_{d} | K_{D} |
|---|---|---|---|---|
| Test article | Target | (M⁻¹s⁻¹) | (s⁻¹) | (nM) |
| F3′-1602 #1 | hCLEC12A-His | 8.54 × 10⁵ | 4.94 × 10⁻⁴ | 0.58 |
| F3′-1602 #2 | hCLEC12A-His | 8.33 × 10⁵ | 4.94 × 10⁻⁴ | 0.59 |
| F3′-1602 #3 | hCLEC12A-His | 8.63 × 10⁵ | 4.89 × 10⁻⁴ | 0.57 |
| F3′-1602 #4 | hCLEC12A-His | 8.26 × 10⁵ | 4.99 × 10⁻⁴ | 0.60 |
| Average ± Std Dev | hCLEC12A-His | (8.44 ± 0.03) × 10⁵ | (4.94 ± 0.09) × 10⁻⁴ | 0.59 ± 0.01 |

Polymorphic variant CLEC12A-K244Q is prevalent in 30% of the population. Binding of F3′-1602 to CLEC12A-K244Q was examined by SPR and compared its affinity to wild-type CLEC12A. As shown in Table 16, binding kinetics of F3′-1602 wild-type and the K244Q variant of CLEC12A are similar.

TABLE 16
Kinetic parameters and affinities of
F3′-1602 for human CLEC12A-K244Q.

| | | kₐ | k_{d} | K_{D} |
|---|---|---|---|---|
| Test articles | Target | (M⁻¹s⁻¹) | (s⁻¹) | (nM) |
| F3′-1602 #1 | hCLEC12A WT | 8.26 × 10⁵ | 4.96 × 10⁻⁴ | 0.60 |
| F3′-1602 #2 | hCLEC12A-K244Q | 5.65 × 10⁵ | 4.36 × 10⁻⁴ | 0.77 |

### Binding Unrelated Recombinant Proteins and Cell Binding Specificity

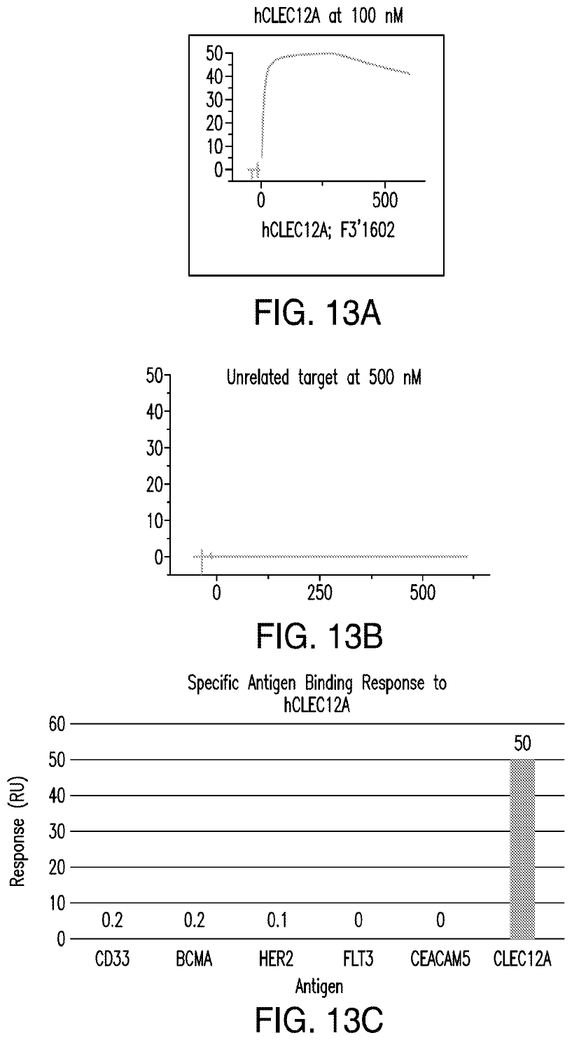

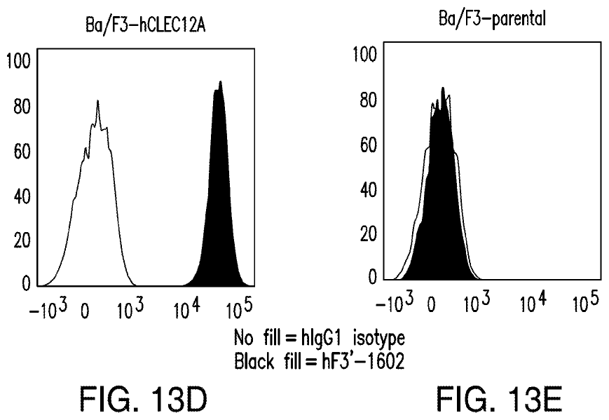

Specificity for CLEC12A was tested by SPR against five different unrelated proteins at concentrations as high as 500 nM and is shown in FIG. 13. No non-specific binding was observed to any of the unrelated recombinant targets (FIG. 13B, FIG. 13C), whereas positive control (CLEC12A) showed binding of 50 RU at 100 nM concentration (FIG. 13A). Specificity was also tested against proteins expressed on the surface of Ba/F3 cell line. No non-nonspecific binding of F3′-1602 at a concentration of 333 nM to Ba/F3 parental cell line was observed (FIG. 13E), whereas Ba/F3 cell lines engineered to express CLEC12A, used as a positive control, showed a significant shift on the FACS plot by flow cytometry (FIG. 13D), suggesting that the binding of F3′-1602 to the latter was CLEC12A-specific.

### Non-Specific Binding to Polyspecific Reagent (PSR)

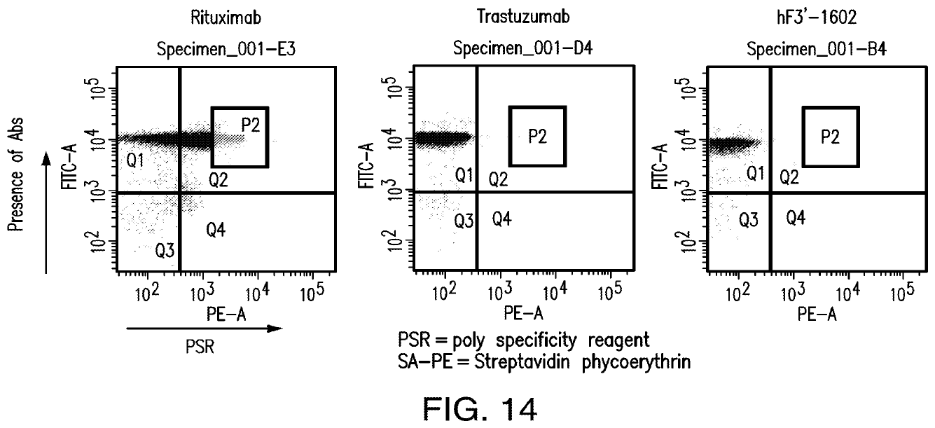

Flow cytometry based PSR assay allows to filter out antibodies that have a higher probability to bind non-specifically to unrelated proteins. As part of the developability assessment, F3′-1602 was tested for non-specific binding to a preparation of detergent solubilized membrane proteins in a PSR assay (FIG. 14). PSR assay correlates well with cross-interaction chromatography, a surrogate for antibody solubility, as well as with baculovirus particle enzyme-linked immunosorbent assay, a surrogate for in vivo clearance (Xu et. al (2013). Addressing polyspecificity of antibodies selected from an in vitro yeast presentation system: a FACS-based, high-throughput selection and analytical tool. Protein engineering design and selection, 26, 663-670).

50 μL of 100 nM F3′-1602 or control mAb in PBSF were incubated with pre-washed 5 μL protein A dyna beads slurry (Invitrogen, catalog #10001D) for 30 minutes at room temperature. Multispecific binding protein or mAb bound magnetic beads were allowed to stand on a magnetic rack for 60 seconds and the supernatant was discarded. The bound beads were washed with 100 μL PBSF. Beads were incubated for 20 minutes on ice with 50 μL of biotinylated PSR reagent which was diluted 25-fold from the stock (Xu et. al. 2013). Samples were put on the magnetic rack, supernatant discarded, and washed with 100 μL of PBSF. A secondary FACS reagent, to detect binding of biotinylated PSR reagent to multispecific binding proteins or control mAbs, was made as follows: 1:250 μL of Streptavidin-PE (Biolegend, catalog #405204) and 1:100 donkey anti-human Fc were combined in PBSF. To each sample, 100 μL of the secondary reagents were added and allowed to incubate for 20 minutes on ice. The beads were washed twice with 100 μL PBSF, and samples were analyzed on a FACS Celesta (BD). Two PSR controls, Rituximab (PSR positive) and Trastuzumab (PSR clean), were used in the assay.

### High-Spec® Cross Reactivity Assay on HuProt™ Human Proteome Assays

To examine specificity of F3′-1602, a protein array technology was used. The HuProt™ human proteome microarray provides the largest database of individually purified human full length proteins on a single microscopic slide. An array consisting of 22,000 full-length human proteins are expressed in yeast S. cerevisiae, purified, and subsequently printed in duplicate on a mircoarray glass slide that allows thousands of interactions to be profiled in a high-throughput manner.

Specificity of F3′-1602 was tested at 0.1 μg/ml and 1 μg/ml concentration against native Huprot human proteome array embedded on microslides at CDI laboratories (Baltimore, Md.), according to their standard procedure.

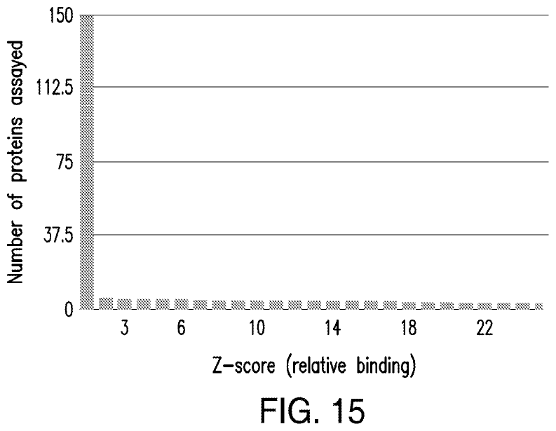

FIG. 15 shows the relative binding (Z score) of F3′-1602 at 1 μg/ml to human CLEC12A in comparison to the entire human proteome microarray. Z score is the average binding score of two duplicates of a given protein. For comparison purpose, the top 24 proteins with residual background binding to F3′-1602 were also provided in FIG. 15. Table 17 shows the Z and S scores of F3′-1602 to human CLEC12A and the top 6 proteins from the microarray. S score is the difference of Z score of a given protein and the one rank next to it. If the S score of the top hit is >3 an antibody is considered to be highly specific to its target. Based on the Z and S score criteria, F3′-1602 showed high specificity to human and lack of off-target binding in the HuProt™ human proteome assay.

TABLE 17
Z and S scores of F3′-1602 in HuProt ™ human
proteome microarray assay.

| Name | Rank | Protein-ID | Z score | S score |
|---|---|---|---|---|
| hCLEC12A | 1 | | 150.21 | 144.62 |
| MFF | 2 | JHU15965.P1168B06 | 5.60 | 0.59 |
| RPLP0 | 3 | JHU16464.P173H05 | 5.01 | 0.12 |
| PARS2 | 4 | JHU06781.P071H01 | 4.89 | 0.062 |
| HMGA1_frag | 5 | JHU16418.P173B08 | 4.83 | 0.27 |
| THEMIS2_frag | 6 | JHU15201.P160B01 | 4.56 | 0.37 |
| HAGHL | 7 | JHU08774.P092D12 | 4.19 | 0.01 |

### Molecular Modeling

Anti-CLEC12A binding arm of F3′-1602 was assessed with 377 post Phase I biotherapeutic molecules using Therapeutic Antibody Profiler (TAP) available at the SAbPred website. TAP used ABodyBuilder to generate a model for F3′-1602 with side chains by PEARS. The CDRH3 was built by MODELLER due to its diversity.

Five different parameters were evaluated:
Total CDR length
Patches of surface hydrophobicity (PSH) across the CDR vicinity
Patches of positive charge (PPC) across the CDR vicinity
Patches of negative charge (PNC) across the CDR vicinity
Structural Fv charge symmetry parameter (sFvCSP)

These parameters of F3′-1602 were then compared with the profile distributions of therapeutic antibodies to predict the developability and any potential issues that might cause downstream challenges.

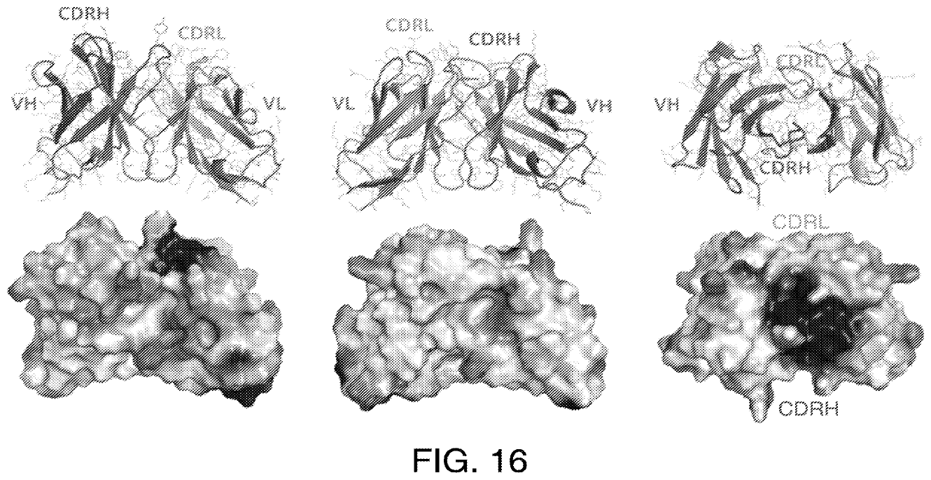

FIG. 16 illustrates a ribbon diagram model of the CLEC12A binding scFv in three different orientations (upper panel) and their corresponding surface charge distribution of the same orientation (lower panel). The charge distribution of anti-CLEC12A scFv is polarized (“top view”, lower panel), with negatively-charged residues populated predominately within CDRH3 and CDRL2. The uneven distribution of electrostatic patches on the paratope is likely to be target-related and reflects the complementarity of charge distribution on its cognate epitope, which may likely contribute to the high affinity interaction between CLEC12A and the scFv of F3′-1602.

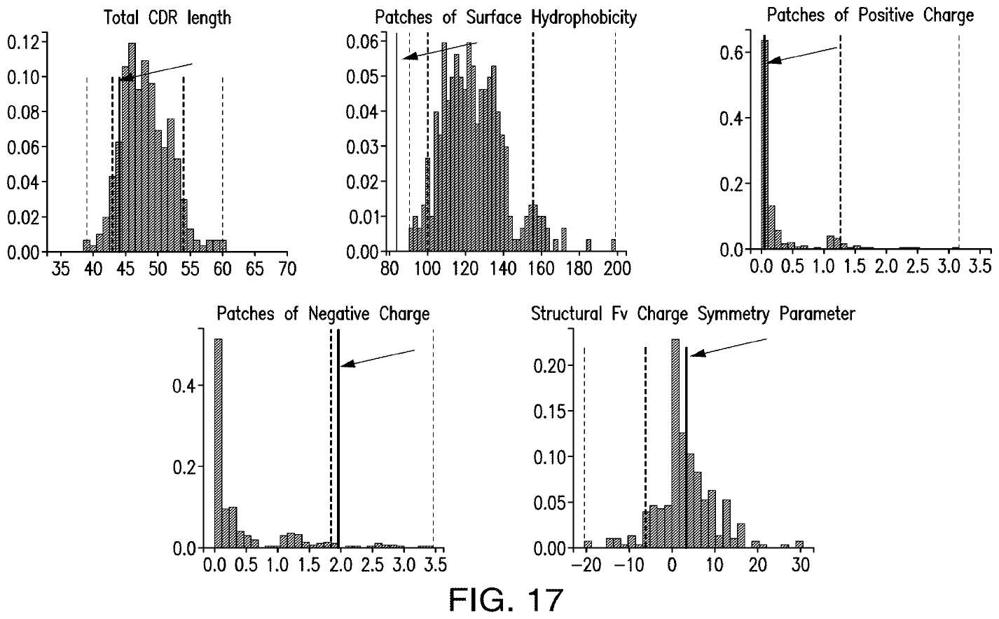

FIG. 17 shows the CDR length and surface hydrophobicity analyses of scFv CLEC12A targeting arm of F3′-1602. The length of CDRs for the CLEC12A binding arm of F3′-1602 are typical for late stage therapeutic antibodies.

The hydrophobicity of a monoclonal antibody is an important biophysical property relevant for its developability into a therapeutic. Hydrophobic patch analysis of the CDRs of an antibody is predictive of its behavior. CLEC12A arm of F3′-1602 has much lower hydrophobicity comparing to other reference molecules. Based on the modeling, there is no hydrophobic patch of significant size on the surface of CLEC12A-binding arm of F3′-1602.

The charge distribution of anti-CLEC12A scFv is polarized, with negatively-charged residues populated predominately within CDRH3 and CDRL2 (as shown in FIG. 16). Although the positively charged patches and charge symmetry are within the norm, without wishing to be bound by theory, it is hypothesized that the distinct negatively charged patches on the paratope is target-related and reflects the complementarity of charge distribution on its cognate epitope, which may contribute to its high affinity interaction with CLEC12A.

### Example 7. F3′-1602 Analysis of Putative Sequence Liabilities

The amino acid sequences of the three polypeptide chains of F3′-1602 was analyzed for putative sequence liabilities as described in Example 3 supra. Putative sequence liabilities are shown in Table 18.

TABLE 18
Putative sequence liabilities in F3′-1602.
CDRs are under Chothia numbering.

| VL-CL | VH-CH1-Fc | VH-VL-Fc |
|---|---|---|
| None | DP in CDRH3 | DS in CDRH3 |
| | (potential chemical | (potential aspartate |
| | instability) | isomerization) |

To examine the liabilities, accelerated stability (4 weeks at 40° C.) and forced degradation studies were performed. The DP in the CDRH3 of VH-CH1-Fc was not modified in accelerated stability studies or forced degradation studies, and therefore, no further analysis was necessary. However, it was observed that modification of the DS in the CDRH3 in the scFv led to a reduction in CLEC12A binding of F3′-1602 in accelerated stability studies.

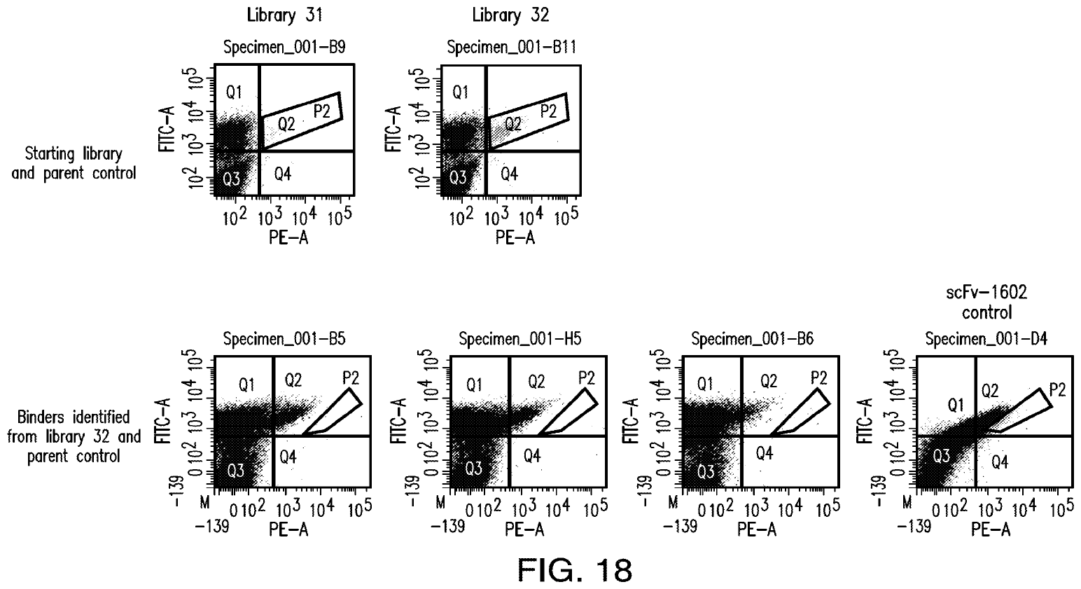

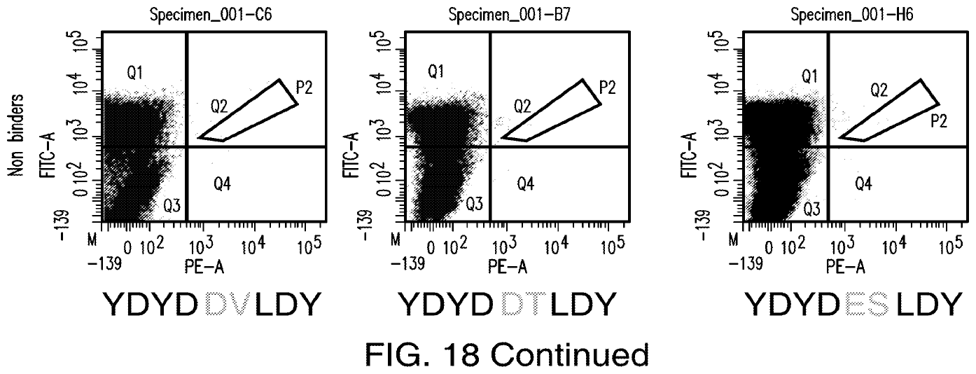

To replace the DS in CDRH3, yeast display was performed to identify alternative sequence motifs without the DS site. Three variants, namely YDYDDALDY (SEQ ID NO:141), YDYDDILDY (SEQ ID NO:142), and YDYDDLLDY (SEQ ID NO:143), were identified that showed binding to hCLEC12A albeit with weaker binding signal compared to the parent scFv (YDYDDSLDY) (SEQ ID NO:5), while variants YDYDDVLDY (SEQ ID NO:144), YDYDDTLDY (SEQ ID NO:145), and YDYDESLDY (SEQ ID NO:146) were identified as non-binders. Based on the binding analysis only variants YDYDDALDY ((SEQ ID NO:141), AB0053) and YDYDDILDY ((SEQ ID NO:142), AB0085) were considered for mammalian production and further characterization. Binding of AB0053 and AB0085 to hCLEC12A-His was characterized using Surface Plasmon Resonance (SPR) at 37° C. Both mutations introduced to remove DS sequence liability had an effect on the binding to hCLEC12A-His (Table 19). FIG. 18 demonstrates FACS analysis of binding to hCLEC12A-his using yeast libraries generated to remove the sequence liability in CDRH3. The data showed complete loss of binding in the DS to DI engineered variant (AB0085). Introduction of DA in place of DS (AB0053) did not eliminate binding completely but lead to apparent heterogeneity in binding sensorgram.

Binding affinities of F3′-1602 to recombinant human CLEC12A were measured by SPR at 37° C. using a Biacore 8K instrument. Briefly, human Fc specific antibodies were covalently immobilized at a density 8000-10000 resonance units (RU) on carboxy methyl dextran matrix of a CM5 biosensor chip via amine-coupling chemistry to create an anti-hFc IgG chip. F3′-1602 samples were captured on the anti-hFc IgG chip at a concentration of 1.5 μg/mL at a flow rate of 10 μL/min for 60 seconds to achieve ˜150-250 RU capture level. hCLEC12A-His was serially diluted (100 nM-0.046 nM) in three-fold dilutions with HBS-EP+ buffer (1×; 10 mM HEPES, 150 mM NaCl, 3 mM EDTA, 0.05% P20 pH 7.4) with 0.1 mg/mL bovine serum albumin (BSA) and injected at a flow rate of 30 μl/min over the captured test articles. Association was monitored for 300 seconds and dissociation was monitored for 600 seconds. Surfaces were regenerated between cycles with three pulses of 10 mM glycine-HCl, pH 1.7 injected for 20 seconds at 100 μl/min. HBS-EP+(1×) with 0.1 mg/ml BSA buffer was used throughout the experiment. Data were analyzed using Biacore 8K Insight Evaluation software (GE Healthcare).

TABLE 19
Kinetic parameters and affinities of DS engineered
clones for hCLEC12A for CLEC12A.

| Test | kₐ | k_{d} | K_{D} | |
|---|---|---|---|---|
| Article | (M⁻¹s⁻¹) | (s⁻¹) | (nM) | Notes |
| AB0053 | 1.63 × 10⁶* | k_{d1} = 1.57 × 10⁻² and | 2.41 | Two-state |
| | | k_{d2} = 9.49 × 10⁻⁴ | | fit |
| AB0085 | No Binding | | | |
| F3′-1602 | 1.11 × 10⁶ | 8.95 × 10⁻⁴ | 0.80 | Classical |
| (AB0237) | | | | 1:1 fit |

*kₐ₂ value was insignificant compared to the kₐ₁ value. Therefore, only kₐ₁ value is shown.

As the library approach produced AB0053 as the only hit and that hit demonstrated kinetics of binding to hCLEC12A significantly different from F3′-1602, it was concluded that the DS motif is necessary for maintaining architecture of CDRH3 and therefore cannot be effectively removed.

### INCORPORATION BY REFERENCE

Unless stated to the contrary, the entire disclosure of each of the patent documents and scientific articles referred to herein is incorporated by reference for all purposes.

### EQUIVALENTS

An antigen-binding site described in application may be embodied in other specific forms without departing from the spirit or essential characteristics thereof. The foregoing embodiments are therefore to be considered in all respects illustrative rather than limiting on an antigen-binding site described herein. Scope of the present application is thus indicated by the appended claims rather than by the foregoing description, and all changes that come within the meaning and the range of equivalency of the claims are intended to be embraced therein.

SEQUENCES

| SEQ | Sequence |
|---|---|
| ID | |
| NO. | |
| 1 | EVQLQESGPGLVQPSQSLSITCTVSGFSLTNYGLHWVRQSPGKGLEWLGVIWSGGKTDYNTPFK |
| | SRLSISKDISKNQVFFKMNSLQPNDTAIYFCAKYDYDDSLDYWGQGTSVTVSS |
| 2 | DIQMNQSPSSLSASLGDTIAITCHASQNINFWLSWYQQKPGNIPKLLIYEASNLHTGVPSRFSG |
| | SGSGTRFTLTISSLQPEDIATYYCQQSHSYPLTFGQGTKLEIK |
| 3 | QLQLQESGPGLVKPSETLSLTCTVSGFSLTNYGLHWIRQPPGKCLEWIGVIWSGGKTDYNPSLK |
| | SRVTISKDTSKNQFSLKLSSVQANDTAVYYCAKYDYDDSLDYWGQGTLVTVSSGGGGSGGGGSG |
| | GGGSGGGGSDIQMTQSPSSLSASVGDRVTITCHASQNINFWLSWYQQKPGKIPKLLIYEASNLH |
| | TGVPSRFSGSGSGTRFTLTISSLQPEDIATYYCQQSHSYPLTFGCGTKLEIK |
| 4 | WSGGK |
| 5 | YDYDDSLDY |
| 6 | HASQNINFWLS |
| 7 | EASNLHT |
| 8 | QQSHSYPLT |
| 9 | QLQLQESGPGLVKPSETLSLTCTVSGFSLTNYGLHWIRQPPGKGLEWIGVIWSGGKTDYNPSLK |
| | SRVTISKDTSKNQFSLKLSSVQANDTAVYYCAKYDYDDSLDYWGQGTLVTVSS |
| 10 | DIQMTQSPSSLSASVGDRVTITCHASQNINFWLSWYQQKPGKIPKLLIYEASNLHTGVPSRFSG |
| | SGSGTRFTLTISSLQPEDIATYYCQQSHSYPLTFGQGTKLEIK |
| 11 | GFSLTNY |
| 12 | DIQMTQSPSSLSASVGDRVTITCHASQNINFWLSWYQQKPGKIPKLLIYEASNLHTGVPSRFSG |
| | SGSGTRFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISKDTSKNQFSLKLSSVQANDTAVYYCAKYDYDDSLDYWGQGTLVTVSS |
| 13 | QLQLQESGPGLVKPSETLSLTCTVSGFSLTNYGLHWIRQPPGKGLEWIGVIWSGGKTDYNPSLK |
| | SRVTISVDTSKNQFSLKLSSVQANDTAVYYCAKYDYDDSLDYWGQGTLVTVSS |
| 14 | EVQLVESGGGVVQPGGSLRLSCAASGFTFNAFGMHWVRQAPGKGLEWVAFISSGSTSIYYANTV |
| | KGRFTISRDNSKNTLYLQMNSLRAEDTAVYYCARDGYPTGGAMDYWGQGTSVTVSS |
| 15 | QLQLQESGPGLVKPSETLSLTCTVSGFSLTNYGLHWIRQPPGKCLEWIGVIWSGGKTDYNPSLK |
| | SRVTISVDTSKNQFSLKLSSVQANDTAVYYCAKYDYDDSLDYWGQGTLVTVSSGGGGSGGGGSG |
| | GGGSGGGGSDIQMTQSPSSLSASVGDRVTITCHASQNINFWLSWYQQKPGKIPKLLIYEASNLH |
| | TGVPSRFSGSGSGTRFTLTISSLQPEDIATYYCQQSHSYPLTFGCGTKLEIK |
| 16 | DIQMTQSPSSLSASVGDRVTITCHASQNINFWLSWYQQKPGKIPKLLIYEASNLHTGVPSRFSG |
| | SGSGTRFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISVDTSKNQFSLKLSSVQANDTAVYYCAKYDYDDSLDYWGQGTLVTVSS |
| 17 | DIQMTQSPSSLSASVGDRVTITCHASQNINFWLSWYQQKPGKX₁PKLLIYEASNLHTGVPSRFS |
| | GSGSGTX₂FTLTISSLQPEDX₃ATYYCQQSHSYPLTFGX₄GTKLEIK |
| | where X₁ is A or I, X₂ is D or R, X₃ is F or I, and X₄ is Q or G |
| 18 | KSSQSLLWNVNQNNYLV |
| 19 | QLQLQESGPGLVKPSETLSLTCTVSGFSLTNYGLHWIRQPPGKCLEWIGVIWSGGKTDYNPSLK |
| | SRVTISKDTSKNQFSLKLSSVTANDTAVYYCAKYDYDDSLDYWGQGTLVTVSSGGGGSGGGGSG |
| | GGGSGGGGSDIQMTQSPSSLSASVGDRVTITCHASQNINFWLSWYQQKPGKIPKLLIYEASNLH |
| | TGVPSRFSGSGSGTRFTLTISSLQPEDIATYYCQQSHSYPLTFGCGTKLEIK |
| 20 | DIQMTQSPSSLSASVGDRVTITCHASQNINFWLSWYQQKPGKIPKLLIYEASNLHTGVPSRFSG |
| | SGSGTRFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISKDTSKNQFSLKLSSVTANDTAVYYCAKYDYDDSLDYWGQGTLVTVSS |
| 21 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPG |
| 22 | EVQLQESGAELVRSGASIKLSCAASAFNIKDYFIHWVRQRPDQGLEWIGWIDPENDDTEYAPKF |
| | QDKATMTADTSSNTAYLQLSSLTSADTAVYYCNALWSRGGYFDYWGQGTTLTVSS |
| 23 | QLQLQESGPGLVKPSETLSLTCTVSGFSLTNYGLHWIRQPPGKCLEWIGVIWSGGKTDYNPSLK |
| | SRVTISKDTSKNQFSLKLSSVQAADTAVYYCAKYDYDDSLDYWGQGTLVTVSSGGGGSGGGGSG |
| | GGGSGGGGSDIQMTQSPSSLSASVGDRVTITCHASQNINFWLSWYQQKPGKIPKLLIYEASNLH |
| | TGVPSRFSGSGSGTRFTLTISSLQPEDIATYYCQQSHSYPLTFGCGTKLEIK |
| 24 | DIQMTQSPSSLSASVGDRVTITCHASQNINFWLSWYQQKPGKIPKLLIYEASNLHTGVPSRFSG |
| | SGSGTRFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISKDTSKNQFSLKLSSVQAADTAVYYCAKYDYDDSLDYWGQGTLVTVSS |
| 25 | EVLLTQSPAIIAASPGEKVTITCSARSSVSYMSWYQQKPGSSPKIWIYGISKLASGVPARFSGS |
| | GSGTYFSFTINNLEAEDVATYYCQQRSYYPFTFGSGTKLEIK |
| 26 | AFNIKDY |
| 27 | QLQLQESGPGLVKPSETLSLTCTVSGFSLTNYGLHWIRQPPGKCLEWIGVIWSGGKTDYNPSLK |
| | SRVTISKDTSKNQFSLKLSSVQANDTAVYYCARYDYDDSLDYWGQGTLVTVSSGGGGSGGGGSG |
| | GGGSGGGGSDIQMTQSPSSLSASVGDRVTITCHASQNINFWLSWYQQKPGKIPKLLIYEASNLH |
| | TGVPSRFSGSGSGTRFTLTISSLQPEDIATYYCQQSHSYPLTFGCGTKLEIK |
| 28 | DIQMTQSPSSLSASVGDRVTITCHASQNINFWLSWYQQKPGKIPKLLIYEASNLHTGVPSRFSG |
| | SGSGTRFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISKDTSKNQFSLKLSSVQANDTAVYYCARYDYDDSLDYWGQGTLVTVSS |
| 29 | EVQLVESGGGVVQPGGSLRLSCAASGFTFNSFGMHWVRQAPGKGLEWVAFISSGSTSIYYANTV |
| | KGRFTISRDNSKNTLYLQMNSLRAEDTAVYYCARDGYPTGGAMDYWGQGTSVTVSS |
| 30 | DIQMTQSPSSLSASVGDRVTITCHASQNINFWLSWYQQKPGKAPKLLIYEASNLHTGVPSRFSG |
| | SGSGTRFTLTISSLQPEDIATYYCQQSHSYPLTFGQGTKLEIK |
| 31 | QLQLQESGPGLVKPSETLSLTCTVSGFSLTNYGLHWIRQPPGKCLEWIGVIWSGGKTDYNPSLK |
| | SRVTISKDTSKNQFSLKLSSVQANDTAVYYCAKYDYDDSLDYWGQGTLVTVSSGGGGSGGGGSG |
| | GGGSGGGGSDIQMTQSPSSLSASVGDRVTITCHASQNINFWLSWYQQKPGKAPKLLIYEASNLH |
| | TGVPSRFSGSGSGTRFTLTISSLQPEDIATYYCQQSHSYPLTFGCGTKLEIK |
| 32 | DIQMTQSPSSLSASVGDRVTITCHASQNINFWLSWYQQKPGKAPKLLIYEASNLHTGVPSRFSG |
| | SGSGTRFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISKDTSKNQFSLKLSSVQANDTAVYYCAKYDYDDSLDYWGQGTLVTVSS |
| 33 | WSGGS |
| 34 | DIQMTQSPSSLSASVGDRVTITCHASQNINFWLSWYQQKPGKIPKLLIYEASNLHTGVPSRFSG |
| | SGSGTDFTLTISSLQPEDIATYYCQQSHSYPLTFGQGTKLEIK |
| 35 | QLQLQESGPGLVKPSETLSLTCTVSGFSLTNYGLHWIRQPPGKCLEWIGVIWSGGKTDYNPSLK |
| | SRVTISKDTSKNQFSLKLSSVQANDTAVYYCAKYDYDDSLDYWGQGTLVTVSSGGGGSGGGGSG |
| | GGGSGGGGSDIQMTQSPSSLSASVGDRVTITCHASQNINFWLSWYQQKPGKIPKLLIYEASNLH |
| | TGVPSRFSGSGSGTDFTLTISSLQPEDIATYYCQQSHSYPLTFGCGTKLEIK |
| 36 | DIQMTQSPSSLSASVGDRVTITCHASQNINFWLSWYQQKPGKIPKLLIYEASNLHTGVPSRFSG |
| | SGSGTDFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISKDTSKNQFSLKLSSVQANDTAVYYCAKYDYDDSLDYWGQGTLVTVSS |
| 37 | DPENDD |
| 38 | DIQMTQSPSSLSASVGDRVTITCHASQNINFWLSWYQQKPGKIPKLLIYEASNLHTGVPSRFSG |
| | SGSGTRFTLTISSLQPEDFATYYCQQSHSYPLTFGQGTKLEIK |
| 39 | QLQLQESGPGLVKPSETLSLTCTVSGFSLTNYGLHWIRQPPGKCLEWIGVIWSGGKTDYNPSLK |
| | SRVTISKDTSKNQFSLKLSSVQANDTAVYYCAKYDYDDSLDYWGQGTLVTVSSGGGGSGGGGSG |
| | GGGSGGGGSDIQMTQSPSSLSASVGDRVTITCHASQNINFWLSWYQQKPGKIPKLLIYEASNLH |
| | TGVPSRFSGSGSGTRFTLTISSLQPEDFATYYCQQSHSYPLTFGCGTKLEIK |
| 40 | DIQMTQSPSSLSASVGDRVTITCHASQNINFWLSWYQQKPGKIPKLLIYEASNLHTGVPSRFSG |
| | SGSGTRFTLTISSLQPEDFATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISKDTSKNQFSLKLSSVQANDTAVYYCAKYDYDDSLDYWGQGTLVTVSS |
| 41 | QLQLQESGPGLVKPSETLSLTCTVSGFSLTNYGLHWIRQPPGKGLEWIGVIWSGGKTDYNPSLK |
| | SRVTISVDTSKNQFSLKLSSVTAADTAVYYCARYDYDDSLDYWGQGTLVTVSS |
| 42 | DIQMTQSPSSLSASVGDRVTITCHASQNINFWLSWYQQKPGKAPKLLIYEASNLHTGVPSRFSG |
| | SGSGTDFTLTISSLQPEDFATYYCQQSHSYPLTFGQGTKLEIK |
| 43 | QLQLQESGPGLVKPSETLSLTCTVSGFSLTNYGLHWIRQPPGKCLEWIGVIWSGGKTDYNPSLK |
| | SRVTISVDTSKNQFSLKLSSVTAADTAVYYCARYDYDDSLDYWGQGTLVTVSSGGGGSGGGGSG |
| | GGGSGGGGSDIQMTQSPSSLSASVGDRVTITCHASQNINFWLSWYQQKPGKAPKLLIYEASNLH |
| | TGVPSRFSGSGSGTDFTLTISSLQPEDFATYYCQQSHSYPLTFGCGTKLEIK |
| 44 | DIQMTQSPSSLSASVGDRVTITCHASQNINFWLSWYQQKPGKAPKLLIYEASNLHTGVPSRFSG |
| | SGSGTDFTLTISSLQPEDFATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISVDTSKNQFSLKLSSVTAADTAVYYCARYDYDDSLDYWGQGTLVTVSS |
| 45 | QLQLQESGPGLVKPSETLSLTCTVSGFSLTNYGLHWIRQPPGKGLEWIGVIWSGGKTDYNPSLK |
| | SRVTISKDTSKNQFSLKLSSVQAADTAVYYCAKYDYDDSLDYWGQGTLVTVSS |
| 46 | QHNHGSFLPYT |
| 47 | QLQLQESGPGLVKPSETLSLTCTVSGFSLTNYGLHWIRQPPGKCLEWIGVIWSGGKTDYNPSLK |
| | SRVTISKDTSKNQFSLKLSSVQAADTAVYYCAKYDYDDSLDYWGQGTLVTVSSGGGGSGGGGSG |
| | GGGSGGGGSDIQMTQSPSSLSASVGDRVTITCHASQNINFWLSWYQQKPGKAPKLLIYEASNLH |
| | TGVPSRFSGSGSGTRFTLTISSLQPEDIATYYCQQSHSYPLTFGCGTKLEIK |
| 48 | DIQMTQSPSSLSASVGDRVTITCHASQNINFWLSWYQQKPGKAPKLLIYEASNLHTGVPSRFSG |
| | SGSGTRFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWSGGKTDYNPSLKS |
| | RVTISKDTSKNQFSLKLSSVQAADTAVYYCAKYDYDDSLDYWGQGTLVTVSS |
| 49 | QLQLQESGPGLVKPSETLSLTCTVSGFSLTNYGLHWIRQPPGKGLEWIGVIWSGGKTDYNPSLK |
| | SRVTISX₁DTSKNQFSLKLSSVX₂AX₃DTAVYYCAX₄YDYDDSLDYWGQGTLVTVSS |
| | where X₁ is V or K, X₂ is T or Q, X₃ is A or N, and X₄ is R or K |
| 50 | LWSRGGYFDY |
| 51 | EVQLVESGGGVVQPGGSLRLSCAASGFTFNSFGMHWVRQAPGKCLEWVAFISSGSTSIYYANTV |
| | KGRFTISRDNSKNTLYLQMNSLRAEDTAVYYCARDGYPTGGAMDYWGQGTSVTVSSGGGGSGGG |
| | GSGGGGSGGGGSDIQMTQSPSSLSASVGDRVTITCKASQDIYNYLSWFQQKPGKAPKPLIYRAN |
| | ILVSGVPSRFSGSGSGQDYTFTISSLQPEDIATYYCLQFDAFPFTFGCGTKLEIK |
| 52 | DIQMTQSPSSLSASVGDRVTITCKASQDIYNYLSWFQQKPGKAPKPLIYRANILVSGVPSRFSG |
| | SGSGQDYTFTISSLQPEDIATYYCLQFDAFPFTFGCGTKLEIKGGGGSGGGGSGGGGSGGGGSE |
| | VQLVESGGGVVQPGGSLRLSCAASGFTFNSFGMHWVRQAPGKCLEWVAFISSGSTSIYYANTVK |
| | GRFTISRDNSKNTLYLQMNSLRAEDTAVYYCARDGYPTGGAMDYWGQGTSVTVSS |
| 53 | SARSSVSYMS |
| 54 | DGYPTGGAMDY |
| 55 | GISKLAS |
| 56 | QQRSYYPFT |
| 57 | EVQLQESGPELEKPGASVRISCKASGYSFTAYNMNWVKQSNGKSLEWIGNIDPSYGDATYNQKF |
| | KGKATLTVDKSSSTAYMQLKSLTSEDSAVYYCARDNYYGSGYFDYWGQGTTLTVSS |
| 58 | SVLMTQTPLSLPVSLGDRASISCRSSQGIVHINGNTYLEWYLQKPGQSPKLLIYKVSNRFSGVP |
| | DRFSGSGSGTDFTLKISRVEAEDLGVYYCFQGSHVPWTFGGGTKLEIK |
| 59 | GFTFNSF |
| 60 | EVQLQESGGGLVQPGGSRKLSCAASGFTFNSFGMHWVRQAPEKGLEWVAFISSGSTSIYYANTV |
| | KGRFTISRDNPKNTLFLQMTSLRSEDTAMYYCARDGYPTGGAMDYWGQGTSVTVSS |
| 61 | DIKMTQSPSSMYASLGERVTITCKASQDIYNYLSWFQLKPGKSPRPLIYRANILVSGVPSKFSG |
| | SGSGQDYSLTINSLEYEDLGIYYCLQFDAFPFTFGSGTKLEIK |
| 62 | GFTFNAF |
| 63 | SSGSTS |
| 64 | GYSFTAY |
| 65 | KASQDIYNYLS |
| 66 | RANILVS |
| 67 | LQFDAFPFT |
| 68 | DPSYGD |
| 69 | DIQMTQSPSSLSASVGDRVTITCKASQDIYNYLSWFQQKPGKAPKPLIYRANILVSGVPSRFSG |
| | SGSGQDYTFTISSLQPEDIATYYCLQFDAFPFTFGSGTKLEIK |
| 70 | EVQLVESGGGVVQPGGSLRLSCAASGFTFNAFGMHWVRQAPGKCLEWVAFISSGSTSIYYANTV |
| | KGRFTISRDNSKNTLYLQMNSLRAEDTAVYYCARDGYPTGGAMDYWGQGTSVTVSSGGGGSGGG |
| | GSGGGGSGGGGSDIQMTQSPSSLSASVGDRVTITCKASQDIYNYLSWFQQKPGKAPKPLIYRAN |
| | ILVSGVPSRFSGSGSGQDYTFTISSLQPEDIATYYCLQFDAFPFTFGCGTKLEIK |
| 71 | DIQMTQSPSSLSASVGDRVTITCKASQDIYNYLSWFQQKPGKAPKPLIYRANILVSGVPSRFSG |
| | SGSGQDYTFTISSLQPEDIATYYCLQFDAFPFTFGCGTKLEIKGGGGSGGGGSGGGGSGGGGSE |
| | VQLVESGGGVVQPGGSLRLSCAASGFTFNAFGMHWVRQAPGKCLEWVAFISSGSTSIYYANTVK |
| | GRFTISRDNSKNTLYLQMNSLRAEDTAVYYCARDGYPTGGAMDYWGQGTSVTVSS |
| 72 | GFSLTSY |
| 73 | DNYYGSGYFDY |
| 74 | EVQLVESGGGVVQPGGSLRLSCAASGFTFNSFGMHWVRQAPGKCLEWVAFISSGSTSIYYANTV |
| | KGRFTISRDNSKNTLYLQMNSLRAEDTAVYYCARSGYPTGGAMDYWGQGTSVTVSSGGGGSGGG |
| | GSGGGGSGGGGSDIQMTQSPSSLSASVGDRVTITCKASQDIYNYLSWFQQKPGKAPKPLIYRAN |
| | ILVSGVPSRFSGSGSGQDYTFTISSLQPEDIATYYCLQFDAFPFTFGCGTKLEIK |
| 75 | DIQMTQSPSSLSASVGDRVTITCKASQDIYNYLSWFQQKPGKAPKPLIYRANILVSGVPSRFSG |
| | SGSGQDYTFTISSLQPEDIATYYCLQFDAFPFTFGCGTKLEIKGGGGSGGGGSGGGGSGGGGSE |
| | VQLVESGGGVVQPGGSLRLSCAASGFTFNSFGMHWVRQAPGKCLEWVAFISSGSTSIYYANTVK |
| | GRFTISRDNSKNTLYLQMNSLRAEDTAVYYCARSGYPTGGAMDYWGQGTSVTVSS |
| 76 | EVQLVESGGGVVQPGGSLRLSCAASGFTFNSFGMHWVRQAPGKGLEWVAFISSGSTSIYYANTV |
| | KGRFTISRDNSKNTLYLQMNSLRAEDTAVYYCARSGYPTGGAMDYWGQGTSVTVSS |
| 77 | RSSQGIVHINGNTYLE |
| 78 | KVSNRFS |
| 79 | SGYPTGGAMDY |
| 80 | FQGSHVPWT |
| 81 | EVQLVESGGGVVQPGGSLRLSCAASGFTFNSFGMHWVRQAPGKCLEWVAFISSGSTSIYYANTV |
| | KGRFTISRDNSKNTLYLQMNSLRAEDTAVYYCARDGYPTGGAMDYWGQGTSVTVSSGGGGSGGG |
| | GSGGGGSGGGGSDIKMTQSPSSLSASVGDRVTITCKASQDIYNYLSWFQQKPGKAPKPLIYRAN |
| | ILVSGVPSRFSGSGSGQDYTLTISSLQPEDIATYYCLQFDAFPFTFGCGTKLEIK |
| 82 | DIKMTQSPSSLSASVGDRVTITCKASQDIYNYLSWFQQKPGKAPKPLIYRANILVSGVPSRFSG |
| | SGSGQDYTLTISSLQPEDIATYYCLQFDAFPFTFGCGTKLEIKGGGGSGGGGSGGGGSGGGGSE |
| | VQLVESGGGVVQPGGSLRLSCAASGFTFNSFGMHWVRQAPGKCLEWVAFISSGSTSIYYANTVK |
| | GRFTISRDNSKNTLYLQMNSLRAEDTAVYYCARDGYPTGGAMDYWGQGTSVTVSS |
| 83 | EVQLQESGAELVRSGASVKLSCTVSGFNIKDYYMHWVKQRPEQGLEWIGWIDPENGDTENVPKF |
| | QGKATMTADTSSNTAYLQLRSLTSEDTAVYYCKSYYYDSSSRYVDVWGAGTTVTVSS |
| 84 | DIKMTQSPSSLSASVGDRVTITCKASQDIYNYLSWFQQKPGKAPKPLIYRANILVSGVPSRFSG |
| | SGSGQDYTLTISSLQPEDIATYYCLQFDAFPFTFGSGTKLEIK |
| 85 | GIVMTQAPLTLSVTIGQPASISCKSSQSLLDSDGKTFLNWFLQRPGQSPKRLISLVSKLDSGVP |
| | DRFTGSGSGTDFTLKLSRVEPEDLGVYYCWQGTHFPYTFGGGTKLEIK |
| 86 | GFNIKDY |
| 87 | GFSLTSF |
| 88 | DPENGD |
| 89 | SYFAMDY |
| 90 | KSSQSLLDSDGKTFLN |
| 91 | LVSKLDS |
| 92 | GASIRES |
| 93 | WQGTHFPYT |
| 94 | EVQLQESGAELMKPGASVKISCRTTGYTFSTYWIEWVKQRPGRGPEWIGELFPGNSDTTLNEKF |
| | TGKATFTADSSSNTAYMQLSSLTSEDSAVYYCARSGYYGSSLDYWGQGTTLTVSS |
| 95 | GIVMTQSPASLSASVGETVTITCRAGENIHSYLAWYQQKQGKSPQLLVYNAKTLAEGVPSRFSG |
| | SGSGTQFSLKINSLQPEDFGSYYCQHHYGTPRTFGGGTKLEIK |
| 96 | GYTFSTY |
| 97 | FPGNSD |
| 98 | SGYYGSSLDY |
| 99 | RAGENIHSYLA |
| 100 | NAKTLAE |
| 101 | QHHYGTPRT |
| 102 | WSGGN |
| 103 | GIVMTQSPSSLAVTAGEKVTMRCKSSQSLLWNVNQNNYLVWYQQKQGQPPKLLIYGASIRESWV |
| | PDRFTGSGSGTDFTLTISNVHAEDLAVYYCQHNHGSFLPYTFGGGTKLEIK |
| 104 | EVQLQESGPGLVQPSQSLSITCTVSGFSLTSFGIHWVRQSPGKGLEWLGVIWSGGNTDSNAAFI |
| | SRLSITKDISKSQVFFKMNSLQVTDTAIYYCARSYFAMDYWGQGTSVTVSS |
| 105 | GASIRQS |
| 106 | KSSQSLLWNVNQNNYLL |
| 107 | THFGMDY |
| 108 | QVQLRQSGPGLVQPSQSLSITCTVSGFSLTSYGVHWVRQSPGKGLEWLGVMWSGGSTDYNAAFM |
| | SRLSISKDNSKSQVFFTMNSLQADDTAIYYCARTHFGMDYWGQGTPVTVSS |
| 109 | GIVMTQSPSSLAVTAGEKVTMRCKSSQSLLWSVNQNNYLLWYQQKQGQPPKLLIYGASIRQSWV |
| | PDRFTGSGSGTDFTLSISNVHAEDLAVYYCQHNHGSFLPYTFGGGTKLEIK |
| 110 | QLQLQESGPGLVKPSETLSLTCTVSGFSLTNYGLHWIRQPPGKGLEWIGVIWSGGKTDYNPSLK |
| | SRVTISKDTSKNQFSLKLSSVTANDTAVYYCAKYDYDDSLDYWGQGTLVTVSS |
| 111 | KSSQSLLWSVNQNNYLL |
| 112 | XGYPTGGAMDY where X is D or S |
| 113 | EVQLQESGPGLVQPSQSLSITCTVSGFSLTSFGVHWVRQSPGKGLEWLGVIWSGGSTDSNAAFI |
| | SRLTITKDNSKSQVFFKMNSLQATDTAIYYCARSYFAMDYWGQGTSVSVSS |
| 114 | DIVMTQSPSSLAVTAGEKVTMRCKSSQSLLWNVNQNNYLLWYQQKQGQPPKLLIYGASIRESWV |
| | PDRFTGSGSGTDFTLTISNVHVEDLAVYYCQHNHGSFLPYTFGGGTKLEIK |
| 115 | EVQLVESGGGVVQPGGSLRLSCAASGFTFNX₁FGMHWVRQAPGKGLEWVAFISSGSTSIYYANT |
| | VKGRFTISRDNSKNTLYLQMNSLRAEDTAVYYCARX₂GYPTGGAMDYWGQGTSVTVSS |
| | where X₁ is S or A and X₂ is D or S |
| 116 | DIX₁MTQSPSSLSASVGDRVTITCKASQDIYNYLSWFQQKPGKAPKPLIYRANILVSGVPSRFS |
| | GSGSGQDYTX₂TISSLQPEDIATYYCLQFDAFPFTFGSGTKLEIK |
| | where X₁ is Q or K and X₂ is F or L |
| 117 | GFTFNXF where X is S or A |
| 118 | EVQLVESGGGVVQPGGSLRLSCAASGFTFNAFGMHWVRQAPGKCLEWVAFISSGSTSIYYANTV |
| | KGRFTISRDNSKNTLYLQMNSLRAEDTAVYYCARDGYPTGGAMDYWGQGTSVTVSSGGGGSGGG |
| | GSGGGGSGGGGSDIKMTQSPSSLSASVGDRVTITCKASQDIYNYLSWFQQKPGKAPKPLIYRAN |
| | ILVSGVPSRFSGSGSGQDYTLTISSLQPEDIATYYCLQFDAFPFTFGCGTKLEIK |
| 119 | DIKMTQSPSSLSASVGDRVTITCKASQDIYNYLSWFQQKPGKAPKPLIYRANILVSGVPSRFSG |
| | SGSGQDYTLTISSLQPEDIATYYCLQFDAFPFTFGCGTKLEIKGGGGSGGGGSGGGGSGGGGSE |
| | VQLVESGGGVVQPGGSLRLSCAASGFTFNAFGMHWVRQAPGKCLEWVAFISSGSTSIYYANTVK |
| | GRFTISRDNSKNTLYLQMNSLRAEDTAVYYCARDGYPTGGAMDYWGQGTSVTVSS |
| 120 | EVQLVESGGGVVQPGGSLRLSCAASGFTFNSFGMHWVRQAPGKCLEWVAFISSGSTSIYYANTV |
| | KGRFTISRDNSKNTLYLQMNSLRAEDTAVYYCARSGYPTGGAMDYWGQGTSVTVSSGGGGSGGG |
| | GSGGGGSGGGGSDIKMTQSPSSLSASVGDRVTITCKASQDIYNYLSWFQQKPGKAPKPLIYRAN |
| | ILVSGVPSRFSGSGSGQDYTLTISSLQPEDIATYYCLQFDAFPFTFGCGTKLEIK |
| 121 | DIKMTQSPSSLSASVGDRVTITCKASQDIYNYLSWFQQKPGKAPKPLIYRANILVSGVPSRFSG |
| | SGSGQDYTLTISSLQPEDIATYYCLQFDAFPFTFGCGTKLEIKGGGGSGGGGSGGGGSGGGGSE |
| | VQLVESGGGVVQPGGSLRLSCAASGFTFNSFGMHWVRQAPGKCLEWVAFISSGSTSIYYANTVK |
| | GRFTISRDNSKNTLYLQMNSLRAEDTAVYYCARSGYPTGGAMDYWGQGTSVTVSS |
| 122 | QLQLQESGPGLVKPSETLSLTCTVSGFSLTNYGLHWIRQPPGKGLEWIGVIWSGGKTDYNPSLK |
| | SRVTISKDTSKNQFSLKLSSVQANDTAVYYCARYDYDDSLDYWGQGTLVTVSS |
| 123 | EVQLVQSGAEVKKPGASVKVSCKASGYTFTSYYMHWVRQAPGQGLEWMGIINPSGGSTSYAQKF |
| | QGRVTMTRDTSTSTVYMELSSLRSEDTAVYYCARGNYGDEFDYWGQGTLVTVSS |
| 124 | DIQMTQSPSSLSASVGDRVTITCRASQSISSYLNWYQQKPGKAPKLLIYAASSLQSGVPSRFSG |
| | SGSGTDFTLTISSLQPEDFATYYCQQSYSTPPTFGQGTKVEIK |
| 125 | DIQMTQSPSSLSASVGDRVTITCRASQSVSTSSYNYMHWYQQKPGKPPKLLIKYASNLESGVPS |
| | RFSGSGSGTDFTLTISSLQPEDFATYYCQHSWEIPLTFGQGTKVEIK |
| 126 | EVQLVQSGAEVKKPGASVKVSCKASGYSFTDYYMHWVRQAPGQGLEWIGRINPYNGAAFYSQNF |
| | KDRVTLTVDTSTSTAYLELSSLRSEDTAVYYCAIERGADLEGYAMDYWGQGTLVTVSS |
| 127 | YYYDSSSRYVDV |
| 128 | QLQLQESGPGLVKPSETLSLTCTVSGFSLTNYGLHWIRQPPGKGLEWIGVIWVGGATDYNPSLK |
| | SRVTISVDTSKNQFSLKLSSVQAADTAVYYCAKGDYGDTLDYWGQGTLVTVSS |
| 129 | DIQMTQSPSSLSASVGDRVTITCHASQNINFWLSWYQQKPGKAPKLLIYEASNLHTGVPSRFSG |
| | SGSGTDFTLTISSLQPEDFATYYCQQSHSYPLTFGSGTKLEIK |
| 130 | WVGGA |
| 131 | GDYGDTLDY |
| 132 | QLQLQESGPGLVKPSETLSLTCTVSGFSLTNYGLHWIRQPPGKCLEWIGVIWVGGATDYNPSLK |
| | SRVTISVDTSKNQFSLKLSSVQAADTAVYYCAKGDYGDTLDYWGQGTLVTVSSGGGGSGGGGSG |
| | GGGSGGGGSDIQMTQSPSSLSASVGDRVTITCHASQNINFWLSWYQQKPGKAPKLLIYEASNLH |
| | TGVPSRFSGSGSGTDFTLTISSLQPEDFATYYCQQSHSYPLTFGCGTKLEIK |
| 133 | DIQMTQSPSSLSASVGDRVTITCHASQNINFWLSWYQQKPGKAPKLLIYEASNLHTGVPSRFSG |
| | SGSGTDFTLTISSLQPEDFATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVIWVGGATDYNPSLKS |
| | RVTISVDTSKNQFSLKLSSVQAADTAVYYCAKGDYGDTLDYWGQGTLVTVSS |
| 134 | QLQLQESGPGLVKPSETLSLTCTVSGFSLTNYGLHWIRQPPGKGLEWIGVILSGGWTDYNPSLK |
| | SRVTISKDTSKNQFSLKLSSVQAADTAVYYCAKGDYGDALDYWGQGTLVTVSS |
| 135 | DIQMTQSPSSLSASVGDRVTITCHASQNINFWLSWYQQKPGKAPKLLIYEASNLHTGVPSRFSG |
| | SGSGTRFTLTISSLQPEDIATYYCQQSHSYPLTFGSGTKLEIK |
| 136 | LSGGW |
| 137 | GDYGDALDY |
| 138 | QLQLQESGPGLVKPSETLSLTCTVSGFSLTNYGLHWIRQPPGKCLEWIGVILSGGWTDYNPSLK |
| | SRVTISKDTSKNQFSLKLSSVQAADTAVYYCAKGDYGDALDYWGQGTLVTVSSGGGGSGGGGSG |
| | GGGSGGGGSDIQMTQSPSSLSASVGDRVTITCHASQNINFWLSWYQQKPGKAPKLLIYEASNLH |
| | TGVPSRFSGSGSGTRFTLTISSLQPEDIATYYCQQSHSYPLTFGCGTKLEIK |
| 139 | DIQMTQSPSSLSASVGDRVTITCHASQNINFWLSWYQQKPGKAPKLLIYEASNLHTGVPSRFSG |
| | SGSGTRFTLTISSLQPEDIATYYCQQSHSYPLTFGCGTKLEIKGGGGSGGGGSGGGGSGGGGSQ |
| | LQLQESGPGLVKPSETLSLTCTVSGFSLTNYGLHWIRQPPGKCLEWIGVILSGGWTDYNPSLKS |
| | RVTISKDTSKNQFSLKLSSVQAADTAVYYCAKGDYGDALDYWGQGTLVTVSS |
| 140 | DIQMTQSPSSLSASVGDRVTITCHASQNINFWLSWYQQKPGKAPKLLIYEASNLHTGVPSRFSG |
| | SGSGTRFTLTISSLQPEDIATYYCQQSHSYPLTFGGGTKLEIK |
| 141 | YDYDDALDY |
| 142 | YDYDDILDY |
| 143 | YDYDDLLDY |
| 144 | YDYDDVLDY |
| 145 | YDYDDTLDY |
| 146 | YDYDESLDY |
| 147 | QLQLQESGPGLVKPSETLSLTCTVSGFSLTNYGLHWIRQPPGKCLEWIGVIWVGGATDYNPSLK |
| | SRVTISVDTSKNQFSLKLSSVQAADTAVYYCAKGDYGDTLDYWGQGTLVTVSS |
| 148 | DIQMTQSPSSLSASVGDRVTITCHASQNINFWLSWYQQKPGKAPKLLIYEASNLHTGVPSRFSG |
| | SGSGTDFTLTISSLQPEDFATYYCQQSHSYPLTFGCGTKLEIK |

What is claimed is:

1. An antigen-binding site that binds CLEC12A, comprising:
(a) a heavy chain variable domain (VH) comprising complementarity-determining region 1 (CDR1), complementarity-determining region 2 (CDR2), and complementarity-determining region 3 (CDR3) comprising the amino acid sequences of SEQ ID NOs: 11, 4, and 5, respectively; and
(b) a light chain variable domain (VL) comprising CDR1, CDR2, and CDR3 comprising the amino acid sequences of SEQ ID NOs: 6, 7, and 8, respectively.

2. The antigen-binding site of claim 1, wherein the VH comprises an amino acid sequence of SEQ ID NO:49, and the VL comprises an amino acid sequence of SEQ ID NO:17.

3. The antigen-binding site of claim 1 or 2, wherein the VH comprises an amino acid sequence at least 90% identical to SEQ ID NO:45, and the VL comprises an amino acid sequence at least 90% identical to SEQ ID NO:140.

4. The antigen-binding site of any one of claims 1-3, wherein the VH comprises the amino acid sequence of SEQ ID NO:45, and the VL comprises the amino acid sequence of SEQ ID NO:140.

5. The antigen-binding site of claim 1 or 2, wherein the VH and the VL comprise the amino acid sequences of SEQ ID NOs: 9 and 10; 13 and 10; 110 and 10; 45 and 10; 122 and 10; 9 and 30; 9 and 34; 9 and 38; or 41 and 42, respectively.

6. An antigen-binding site that binds CLEC12A, comprising:
(a) a VH comprising CDR1, CDR2, and CDR3 comprising the amino acid sequences of SEQ ID NOs: 117, 63, and 112, respectively; and
(b) a VL comprising CDR1, CDR2, and CDR3 comprising the amino acid sequences of SEQ ID NOs: 65, 66, and 67, respectively.

7. The antigen-binding site of claim 6, wherein the VH comprises CDR1, CDR2, and CDR3 of SEQ ID NO: 59, 63, and 79, respectively; and the VL comprises CDR1, CDR2, and CDR3 of SEQ ID NO: 65, 66, and 67, respectively.

8. The antigen-binding site of claim 6, wherein the VH comprises CDR1, CDR2, and CDR3 of SEQ ID NO: 59, 63, and 54, respectively, and the VL comprises CDR1, CDR2, and CDR3 of SEQ ID NO: 65, 66, and 67, respectively.

9. The antigen-binding site of claim 6, wherein the VH comprises CDR1, CDR2, and CDR3 of SEQ ID NO: 62, 63, and 54, respectively, and the VL comprises CDR1, CDR2, and CDR3 of SEQ ID NO: 65, 66, and 67, respectively.

10. The antigen-binding site of any one of claims 6-9, wherein the VH comprises the amino acid sequence of SEQ ID NO:115, and the VL comprises the amino acid sequence of SEQ ID NO:116.

11. The antigen-binding site of claim 10, wherein the VH and the VL comprise the amino acid sequences of SEQ ID NOs: 29 and 69; 14 and 69; 76 and 69; 29 and 84; 14 and 84; or 76 and 84, respectively.

12. An antigen-binding site that binds CLEC12A, comprising:
(a) a VH comprising CDR1, CDR2, and CDR3 comprising the amino acid sequences of SEQ ID NOs: 87, 33, and 89, respectively; and
(b) a VL comprising CDR1, CDR2, and CDR3 comprising the amino acid sequences of SEQ ID NOs: 106, 92, and 46, respectively.

13. An antigen-binding site that binds CLEC12A, comprising:
(a) a VH comprising CDR1, CDR2, and CDR3 comprising the amino acid sequences of SEQ ID NOs: 72, 33, and 107, respectively; and
(b) a VL comprising CDR1, CDR2, and CDR3 comprising the amino acid sequences of SEQ ID NOs: 111, 105, and 46, respectively.

14. An antigen-binding site that binds CLEC12A, comprising:
(a) a VH comprising CDR1, CDR2, and CDR3 comprising the amino acid sequences of SEQ ID NOs: 87, 102, and 89, respectively; and
(b) a VL comprising CDR1, CDR2, and CDR3 comprising the amino acid sequences of SEQ ID NOs: 18, 92, and 46, respectively.

15. An antigen-binding site that binds CLEC12A, comprising:
(a) a VH comprising CDR1, CDR2, and CDR3 comprising the amino acid sequences of SEQ ID NOs: 26, 37, and 50, respectively; and
(b) a VL comprising CDR1, CDR2, and CDR3 comprising the amino acid sequences of SEQ ID NOs: 53, 55, and 56, respectively.

16. An antigen-binding site that binds CLEC12A, comprising:
(a) a VH comprising CDR1, CDR2, and CDR3 comprising the amino acid sequences of SEQ ID NOs: 64, 68, and 73, respectively; and
(b) a VL comprising CDR1, CDR2, and CDR3 comprising the amino acid sequences of SEQ ID NOs: 77, 78, and 80, respectively.

17. An antigen-binding site that binds CLEC12A, comprising:
(a) a VH comprising CDR1, CDR2, and CDR3 comprising the amino acid sequences of SEQ ID NOs: 86, 88, and 127, respectively; and
(b) a VL comprising CDR1, CDR2, and CDR3 comprising the amino acid sequences of SEQ ID NOs: 90, 91, and 93, respectively.

18. An antigen-binding site that binds CLEC12A, comprising:
(a) a VH comprising CDR1, CDR2, and CDR3 comprising the amino acid sequences of SEQ ID NOs: 96, 97, and 98, respectively; and
(b) a VL comprising CDR1, CDR2, and CDR3 comprising the amino acid sequences of SEQ ID NOs: 99, 100, and 101, respectively.

19. An antigen-binding site that competes with the antigen-binding site of any one of claims 13-17.

20. The antigen-binding site of any one of claims 1-11, 14-15, and 17, wherein the antigen-binding site binds human CLEC12A with a dissociation constant (K_{D}) smaller than or equal to 20 nM as measured by surface plasmon resonance (SPR).

21. The antigen-binding site of any one of claims 1-4, wherein the antigen-binding site binds human CLEC12A with a K_{D} smaller than or equal to 1 nM as measured by SPR.

22. The antigen-binding site of any one of claims 1-11, wherein the antigen-binding site binds CLEC12A in a glycosylation independent manner.

23. The antigen-binding site of any one of claims 1-5 and 20-22, wherein the antigen-binding site binds human CLEC12A comprising a K244Q mutation.

24. The antigen-binding site of any one of claims 1-23, wherein the antigen-binding site is present as a single-chain fragment variable (scFv).

25. The antigen-binding site of claim 24, wherein the scFv comprises an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, 82, 118, 119, 120, 121, 132, 133, 138, and 139.

26. An antigen-binding site that binds CLEC12A in a glycosylation independent manner.

27. A protein comprising the antigen-binding site of any one of the claims 1-26.

28. The protein of claim 27, further comprising an antibody heavy chain constant region.

29. The protein of claim 28, wherein the antibody heavy chain constant region is a human IgG heavy chain constant region.

30. The protein of claim 29, wherein the antibody heavy chain constant region is a human IgG1 heavy chain constant region.

31. The protein of claim 29 or 30, wherein each polypeptide chain of the antibody heavy chain constant region comprises an amino acid sequence at least 90% identical to SEQ ID NO:21.

32. The protein of any one of claims 29-31, wherein at least one polypeptide chain of the antibody heavy chain constant region comprises one or more mutations, relative to SEQ ID NO:21, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

33. The protein of any one of claims 29-32, wherein at least one polypeptide chain of the antibody heavy chain constant region comprises one or more mutations, relative to SEQ ID NO:21, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

34. The protein of any one of claims 29-33, wherein one polypeptide chain of the antibody heavy chain constant region comprises one or more mutations, relative to SEQ ID NO:21, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:21, at one or more positions selected from Q347, Y349, L351, S354, E356, E357, S364, T366, L368, K370, N390, K392, T394, D399, D401, F405, Y407, K409, T411, and K439, numbered according to the EU numbering system.

35. The protein of claim 34, wherein one polypeptide chain of the antibody heavy chain constant region comprises K360E and K409W substitutions relative to SEQ ID NO:21; and the other polypeptide chain of the antibody heavy chain constant region comprises Q347R, D399V and F405T substitutions relative to SEQ ID NO:21, numbered according to the EU numbering system.

36. The protein of claim 34 or 35, wherein one polypeptide chain of the antibody heavy chain constant region comprises a Y349C substitution relative to SEQ ID NO:21; and the other polypeptide chain of the antibody heavy chain constant region comprises an S354C substitution relative to SEQ ID NO:21, numbered according to the EU numbering system.

37. An antibody-drug conjugate comprising the protein of any one of claims 27-36 and a drug moiety.

38. The antibody-drug conjugate of claim 37, wherein the drug moiety is selected from the group consisting of auristatin, N-acetyl-γ calicheamicin, maytansinoid, pyrrolobenzodiazepine, and SN-38.

39. An immunocytokine comprising the antigen-binding site of any one of claims 1-26 and a cytokine.

40. The immunocytokine of claim 39, wherein the cytokine is selected from the group consisting of IL-2, IL-4, IL-10, IL-12, IL-15, TNF, and IFNα.

41. A bispecific T-cell engager comprising the antigen-binding site of any one of claims 1-26 and an antigen-binding site that binds CD3.

42. A chimeric antigen receptor (CAR) comprising:
(a) the antigen-binding site of any one of claims 1-26;
(b) a transmembrane domain; and
(c) an intracellular signaling domain.

43. The CAR of claim 42, wherein the transmembrane domain is selected from the transmembrane regions of the alpha, beta or zeta chain of the T-cell receptor, CD28, CD3 epsilon, CD45, CD4, CD5, CD8, CD9, CD16, CD22, CLEC12A, CD37, CD64, CD80, CD86, CD134, CD137, CD152, and CD154.

44. The CAR of claim 42 or 43, wherein the intracellular signaling domain comprises a primary signaling domain comprising a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

45. The CAR of any one of claims 42-44, wherein the intracellular signaling domain further comprises a costimulatory signaling domain comprising a functional signaling domain of a costimulatory receptor.

46. The CAR of claim 45, wherein the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

47. An isolated nucleic acid encoding the CAR of any one of claims 42-46.

48. An expression vector comprising the isolated nucleic acid of claim 47.

49. An immune effector cell comprising the nucleic acid of claim 47 or the expression vector of claim 48.

50. An immune effector cell expressing the CAR of any one of claims 42-46.

51. The immune effector cell of claim 49 or 50, wherein the immune effector cell is a T cell.

52. The immune effector cell of claim 51, wherein the T cell is a CD8⁺ T cell, a CD4⁺ T cell, or an NKT cell.

53. The immune effector cell of claim 49 or 50, wherein the immune effector cell is an NK cell.

54. A pharmaceutical composition comprising the protein of any one of claims 27-36, the antibody-drug conjugate of claim 37 or 38, the immunocytokine of claim 39 or 40, the bispecific T-cell engager of claim 41, or the immune effector cell of any one of claims 49-53; and a pharmaceutically acceptable carrier.

55. A method of treating cancer, the method comprising administering to a subject in need thereof an effective amount of the protein of any one of claims 27-36, the antibody-drug conjugate of claim 37 or 38, the immunocytokine of claim 39 or 40, the bispecific T-cell engager of claim 41, the immune effector cell of any one of claims 49-53, or the pharmaceutical composition of claim 54.

56. The method of claim 55, wherein the cancer is a hematologic malignancy.

57. The method of claim 56, wherein the hematologic malignancy is selected from the group consisting of acute myeloid leukemia (AML), myelodysplastic syndrome (MDS), acute lymphoblastic leukemia (ALL), myeloproliferative neoplasms (MPNs), lymphoma, non-Hodgkin lymphomas, and classical Hodgkin lymphoma.

58. The method of claim 57, wherein the AML is selected from undifferentiated acute myeloblastic leukemia, acute myeloblastic leukemia with minimal maturation, acute myeloblastic leukemia with maturation, acute promyelocytic leukemia (APL), acute myelomonocytic leukemia, acute myelomonocytic leukemia with eosinophilia, acute monocytic leukemia, acute erythroid leukemia, acute megakaryoblastic leukemia (AMKL), acute basophilic leukemia, acute panmyelosis with fibrosis, and blastic plasmacytoid dendritic cell neoplasm (BPDCN).

59. The method of claim 57 or 58, wherein the AML is characterized by expression of CLEC12A on the AML leukemia stem cells (LSCs).

60. The method of claim 59, wherein the LSCs further express a membrane marker selected from CD34, CD38, CD123, TIM3, CD25, CD32, and CD96.

61. The method of any one of claims 57-60, wherein the AML is a minimal residual disease (MRD).

62. The method of claim 61, wherein the MRD is characterized by the presence or absence of a mutation selected from FLT3-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)).

63. The method of claim 57, wherein the MDS is selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U).

64. The method of claim 57, wherein the MDS is a primary MDS or a secondary MDS.

65. The method of claim 57, wherein the ALL is selected from B-cell acute lymphoblastic leukemia (B-ALL) and T-cell acute lymphoblastic leukemia (T-ALL).

66. The method of claim 57, wherein the MPN is selected from polycythaemia vera, essential thrombocythemia (ET), and myelofibrosis.

67. The method of claim 57, wherein the non-Hodgkin lymphoma is selected from B-cell lymphoma and T-cell lymphoma.

68. The method of claim 57, wherein the lymphoma is selected from chronic lymphocytic leukemia (CLL), lymphoblastic lymphoma (LPL), diffuse large B-cell lymphoma (DLBCL), Burkitt lymphoma (BL), primary mediastinal large B-cell lymphoma (PMBL), follicular lymphoma, mantle cell lymphoma, hairy cell leukemia, plasma cell myeloma (PCM) or multiple myeloma (MM), mature T/NK neoplasms, and histiocytic neoplasms.

69. The method of any one of claims 55-68, wherein the cancer expresses CLEC12A.
